SEQUENCE LISTING (76 sequences)

SEQ ID NO: 1 - DNA, 5193 bp, synthetic construct
  source 1..5193: mol_type = other DNA
gtgagaggacgtatagggaggaggggttcactagaggtgaggctcaagccattagcctgcctaaaccaaccaggctggacagccatcaccaggaaatggatctcagcccagaagatcgaaagttgttcttctcccttctggagatttctatgtcctttacactcattggttaatatcctgggttggattcccacacatcttgacaaacagagacaattgagtatcaccagccaaaagtcatacccaaaaacagcctggcatgacctcacaccagactcaaacttaccctacctttatcctggtggcttctcatctccagaccccagtaacacatagctttctctccacagtgcccagggattgtggttgtaagccttgcatatgtacaggtaagtcagtaggcctttcaccctgaccccagatgcaacaagtggccatgttagagggtggcccaggtattgacctatttccacctttcttcttcatccttagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggtgagagctgcagtgtgtgacatagaagctgcaatagtcagtccatagacagagcttggcataacagacccctgccttgtccatgacctctgtgctaaccaatctctttacccacccacaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgatcccagtgtccttggagccctctggtcctacaggactctgacacctacctccacccctccctgtgtaaataaagcacccagcactgccttgggaccctgcaataatgtcctggtgatttctgagatgtagagtctagctaggtcatggaatgaggggtctccatggtttgaggcctgagttgtgactaaggaaaaacccataggcctacactgccacacccagcacttttgaatttgcctgacatgaaaagaatttacctctccctggaaagtggagccttatccctaggcagttcccttaccagaccttcctctagcttgcactttgttctgggcacagaatgtgtctaaccccccaaagcaaggaagacacaacctctacctccctcactctgtccttaccccttttcctggctaagcatctcactgagtgcgctgaatagatgcatgtggccacagtcttgcagacagacccttgccatctctccactcagctttccagaggctaagtctagcccgtatggtgataatgcagggagctctatgctatctcagtgctatcagactcccaagtggaggatgaacatggacccattaaaaccaacctgcgcagcaacaccctgccaataaggcccgtatgtgaaaatgtgcacacatctacacatgcacaggcacacacacacacacatgcatgggcacacacacatacagagagagagaatcacagaaactcccatgagcatcctatacagtactcaaagataaaaaggtaccaggtctacccacatgatcatcctcggcatttacaagtgggccaactgatacagataaaacttttctatgccaaggacgccaacatatacacaagtccgctcatgacaaatctgtccctgaacctcagactggcgcccgtgactcatacagtggacactcctccaaagctgtatagcttcctttacttccctgtgtgtactttctctgaagtacactcatcacacagaagaggccctgtgattactctggccctctgttcttggtcatcagagaatagacagaagatcaggcaaactacacagacacttcccacaatcatcacaggccctgactctgctctccagtctcaaaactgaaggctggagcacacagaataagctcctgcacaggccaggccagtatcgggtccagtgtgtctgactgagcccagggacaaaatggcagcactttggggaactgaggtttctggtccaagaaggagagatggaggcccagggagggtctgctgacccagcccagcccagcccagctgcagctttctcctgggcctccatacagcctcctgccacacagggaatggccctagccccaccttattgggacaaacactgaccgccctctctgtccagggctgcaactggacgagacctgtgctgaggcccaggacggggagctggacgggctctggacgaccatcaccatcttcatcagcctcttcctgctcagtgtgtgctacagcgctgctgtcacactcttcaaggtcagccatactgtccccacagtgtctacaatgtcctcatactcttccccatactgtccctgtggtgacctataccccacactgtcccatgctaatgaccacagtcttacatgctatgtaatgctgtctacccttctgtatgcacagtctcacaatgtcccatgcagtctccacgatgctccatactgtccccattccaacccatgctgccccttgttccccgctatgctgtcccatgctattgtctgtattttcatgctcttttcacactgtccctagtgtcacattctgcccatgttgtccaccacattgtccccactctgtacacagcctcacactgtaccctgctacccgataatgttccctgttgtccccaactctctccctgcaccatttgtcaactgtcccctgaattcccatgttgttcccacactgttagtgtgtaatgtgctctgtcccaggtgtaccttgttccgtgctgtctcacttcatcgcccattctgtccttgtactaaccccactctatcaccacactgtccctatgcactgcccacattgtcctcatactgtcccattttgtatcttcatcctgtccccatagtgtccaatgatctaccccacactattcccacttcatgcccctacaatttccctattccattcctctctggtcaccatgccatccttcccactcctgcacagctggagagggactcccgggatgagtccttgcccagatgagctacctatctagaggagtcttcaggtgggaagggaatgcagtcttgatcttggtcttattcaccctgtctcacaggtaaagtggatcttctcctcggtggtggagctgaagcagacactggttcctgaatacaagaacatgattgggcaagcaccctaggccacctcctgtaatggcatttcccaggccccgaaggaccctgtccaatatgccaagcagcacaactgagatcacactgtctgctcatctcgctttcctccgaccccgagactcagctactctcaaattttccctctctgaaggaccatgtggacattacattgctccaggccacagccaccaggacctaaaacaccatcacagcagcaccaaagacactggatagacccacaagggcaatagtttcctcaacagtatatccaaactgttgggacaaacgagcaatcactgaagaagtgacaagttcccacaatgtcagtgtccagctgagaagggacaaaaagtggtaccagccctgtccacaccaccttctaattcacaggaatacgtgatagaagaggcaggttgtagatccgaaagatgagacagattttatcaactccagaaagagctgggtccaactgaattattctagcgaccttggcattgtcatgacctgccatgaccttcctccttaacacttcgataaaccctgggatatggaaaatgcctgtgtttctcagggtttgggaaagaaccatccatgttgggattcttgtgtagatcctccttctggtcacagatgcaatacactggattttcaggcaaaggagcaaattcacagacaactctggccctacagccctaagacctagacaccaccatctccttggaattatcaaatttaacacccggcacacaacaaagaaggactgggactttgaggcctttgtgtagccctagagggggcagaggccactgagcagggattgggtgatcacaaggacctcctggagagggacctgaggagcaggttccaattgggccaaagaaagaagaacaacaatagagatgaaggatgctggaaagagccatggtacagcagtcttgtccttcagacatgactcttacagcccaggactcttacagtagctagctggagcagaagtccaagggattaccatgccctagggccacaggctactggagggtggagtgagtctactacacaggtccaatgcctgtttctccattgcttctcagccaatgagaaatcagagtctccacctccaagaaaaaggaaggtggaaatgaaaggtgagcacctgccttcccgtgactggcagaaagatctccacggactcaaggctttgacttcaaacttcccgtagatccctgatgtctttaaggactctgtctgctttgttggttttgtttgtttgttttgtactttgtcaataaaacatttttcaaatattctacaattgctgtgctcttcctatgcaaactggccctggcacttcaaaacatggcacagttaagttgaccagtgggccatgcagagcatactacccctcctggtcctgtgtcctaccagatatcctctaagtgtcccttcactgctgggctccagttctgctgccctgaaccacacaggtcctcagtctgtcctccctatggg

SEQ ID NO: 2 - DNA, 52 bp, synthetic construct
  source 1..52: mol_type = other DNA
gtggttctggctacaagatagagctctgtcaatgatgtttgcagagactaca

SEQ ID NO: 3 - DNA, 58 bp, synthetic construct
  source 1..58: mol_type = other DNA
ctccctatacgtcctctcacctacaagaaaaagtatatgtgattacactgtcagacag

SEQ ID NO: 4 - DNA, 57 bp, synthetic construct
  source 1..57: mol_type = other DNA
gtgtaatcacatatactttttcttgtaggtgagaggacgtatagggaggaggggttc

SEQ ID NO: 5 - DNA, 49 bp, synthetic construct
  source 1..49: mol_type = other DNA
cgtctagtccttgcccacgtgtcgaccccatagggaggacagactgagg

SEQ ID NO: 6 - DNA, 44 bp, synthetic construct
  source 1..44: mol_type = other DNA
tctgaactacttcgtcgacgtgagaggacgtatagggaggaggg

SEQ ID NO: 7 - DNA, 43 bp, synthetic construct
  source 1..43: mol_type = other DNA
cacgtggatccgcggccgcccatagggaggacagactgaggac

SEQ ID NO: 8 - DNA, 104 bp, synthetic construct
  source 1..104: mol_type = other DNA
ttcctgtggctagaaggcagctccatagccctgctgcagtttcgtcctgtataccaggttcacctactaccatatctagccctgcctgcctaaactgagatggg

SEQ ID NO: 9 - DNA, 103 bp, synthetic construct
  source 1..103: mol_type = other DNA
aggcaagaagacagattcttacccctccatttctcttttatccctctctggtcctgaggacgtatagggaggaggggttcactagaggtgaggctcaagccat

SEQ ID NO: 10 - DNA, 119 bp, synthetic construct
  source 1..119: mol_type = other DNA
ctgacaggaggcaagaagacagattcttacccctccatttctcttttatccctctctggtcctaaccaaaccctcccagcaggggtgcccaggcccaggcatggcccagagggagcagc

SEQ ID NO: 11 - DNA, 40 bp, synthetic construct
  source 1..40: mol_type = other DNA
atccctctctggtcctaaccaaaccctcccagcaggggtg

SEQ ID NO: 12 - DNA, 52 bp, synthetic construct
  source 1..52: mol_type = other DNA
ttgacccatctcagtttacatggtgaatgactacaatatatctggaatttgg

SEQ ID NO: 13 - DNA, 4959 bp, synthetic construct
  source 1..4959: mol_type = other DNA
aaccaaaccctcccagcaggggtgcccaggcccaggcatggcccagagggagcagcggggtggggcttaggccaagctgagctcacaccttgacctttcattccagctgtcgcagagatgaaccccaatgtaaatgtgttcgtcccaccacgggatggcttctctggccctgcaccacgcaagtctaaactcatctgcgaggccacgaacttcactccaaaaccgatcacagtatcctggctaaaggatgggaagctcgtggaatctggcttcaccacagatccggtgaccatcgagaacaaaggatccacaccccaaacctacaaggtcataagcacacttaccatctctgaaatcgactggctgaacctgaatgtgtacacctgccgtgtggatcacaggggtctcaccttcttgaagaacgtgtcctccacatgtgctgccagtgagtggcctgggctaagcccaatgcctagccctcccagattagggaagtcctcctacaattatggccaatgccacccagacatggtcatttgctccttgaactttggctccccagagtggccaaggacaagaatgagcaataggcagtagaggggtgagaatcagctggaaggaccagcatcttcccttaagtaggtttgggggatggagactaagcttttttccaacttcacaactagatatgtcataacctgacacagtgttctcttgactgcaggtccctccacagacatcctaaccttcaccatccccccctcctttgccgacatcttcctcagcaagtccgctaacctgacctgtctggtctcaaacctggcaacctatgaaaccctgaatatctcctgggcttctcaaagtggtgaaccactggaaaccaaaattaaaatcatggaaagccatcccaatggcaccttcagtgctaagggtgtggctagtgtttgtgtggaagactggaataacaggaaggaatttgtgtgtactgtgactcacagggatctgccttcaccacagaagaaattcatctcaaaacccaatggtaggtatccccccttcccttcccctccaattgcaggacccttcctgtacctcatagggagggcaggtcctcttccaccctatcctcactactgtcttcatttacagaggtgcacaaacatccacctgctgtgtacctgctgccaccagctcgtgagcaactgaacctgagggagtcagccacagtcacctgcctggtgaagggcttctctcctgcagacatcagtgtgcagtggcttcagagagggcaactcttgccccaagagaagtatgtgaccagtgccccgatgccagagcctggggccccaggcttctactttacccacagcatcctgactgtgacagaggaggaatggaactccggagagacctatacctgtgttgtaggccacgaggccctgccacacctggtgaccgagaggaccgtggacaagtccactggtaaacccacactgtacaatgtctccctgatcatgtctgacacaggcggcacctgctattgaccatgctagcgctcaaccaggcaggccctgggtgtccagttgctctgtgtatgcaaactaaccatgtcagagtgagatgttgcattttataaaaattagaaataaaaaaaatccattcaaacgtcactggttttgattatacaatgctcatgcctgctgagacagttgtgttttgcttgctctgcacacaccctgcatacttgcctccaccctggcccttcctctaccttgccagtttcctccttgtgtgtgaactcagtcaggcttacaacagacagagtatgaacatgcgattcctccagctacttctagatatatggctgaaagcttgcctaacctggtgcaggcagcattcaggcacatatatagacacacatgcatttatacatagatatataggtacacatgtgtagacacatacatgaatgtgtattcatggacacacagacaaaggtacacatatatacacatgagttcatgcgcacacacatgcatggacacttacaaacgccttcagagacaaataggcatagacacacaaccactcacagaaacagataccaatatgcatggtcctgtgtacacagaaacagactataggcaaatatacacaaataaactatatagatacaaagatatgcatatacacacatgtacagaaacatcttcacatgtgtacactaacatgtgaacaggtatagcacacagatacacctggactctgaccagggctgtaatctccaaggctcacggctcagagagcctacactaggctgggtcactgatactcctcaggagcccactctatgattgggagagataaccccaggtacaaagtatgcctatctgtctcaacaccatggggcagaagatactccactaaccacccatgacagaaagttagccttggctgtgtctccattaatagaacacctcagaagaccaatgtgaaattgcctaacccactcacacccaccctgatctccagttcaaaatgcagaaaacataatgcagttgtccaaaagatgccccaaccacacacacacacacacacacacacacacacacacacacacacacacacacacacacacaccatcaaggagcctctgtaaggagtcaccacccaataacactgcctctttgggctcatatcctggacattcttcatattcatatccatttggggcctaggctttagatatccccaagggctcatctttacagggatcagagatcccaataaatgccctggtcccacagcctccctcaggtatctgtctgtttatctcttggtaccaagacccaacattgctggcaggggtaggacaagcaacgcacgggaactctgatcaaagaaagtcatgagatgcctgagtccttcaggaagtaaggagggacaacctctggtatccctgttcttattgctaaagcccaagagacagggagacctgctctaaattctcagtctaaacagcaccgatggcaccacctgctcagggaaagtccagagcacaccaatatcattttgccacagttcctgagtctgcctttacccaggtccatacattgcatctgtcttgcttgctctgctgccccagggctcctggaacaaaggctccaaattagtgtgtcctacagcttggcctgttctgtgcctccgtctagcttgagctattaggggaccagtcaatactcgctaagattctccagaaccatcagggcaccccaacccttatgcaaatgctcagtcaccccaagacttggcttgaccctccctctctgtgtcccttcatagagggggaggtgaatgctgaggaggaaggctttgagaacctgtggaccactgcctccaccttcatcgtcctcttcctcctgagcctcttctacagcaccaccgtcaccctgttcaaggtagtgtggttgtggggctgaggacacagggctgggacagggagtcaccagtcctcactgcctctacctctactccctacaagtggacagcaattcacactgtctctgtcacctgcaggtgaaatgactctcagcatggaaggacagcagagaccaagagatcctcccacagggacactacctctgggcctgggatacctgactgtatgactagtaaacttattcttacgtctttcctgtgttgccctccagcttttatctctgagatggtcttctttctagactgaccaaagactttttgtcaacttgtacaatctgaagcaatgtctggcccacagacagctgagctgtaaacaaatgtcacatggaaataaatactttatcttgtgaactcactttattgtgaaggaatttgttttgtttttcaaacctttcctgcggtgttgacagcccaaggattatctgaatagagcttaggaactggaaatggaacagtgcagtctgatggtacttaagggagaaagagggaaaggaggtgtggaagaagaaaaaagagaagcagagggggaggggagaagggagagggagagggagagggagagggagagggagagggagagggagagagagagagagagagagagagagagagagagagagagagagcatgcactctaacagcaaagtacaacacaggcagccaatggatagcactctggttatctaccctgatggaagaagggaagtagggcagagaaaattccaggcctaatctcccaaaagcaacagaacctggaaactagcctctagccttaggtctctgctctgtccccagcccaccatcttgggctggtgttgcttcaagctagtaatttaggtcttatcccaaagctttgtggtatgtgggtgtgcctttggggagttggctgagattttgaagatgtttgtacctctcccacaacatgacaagccctaggggttagtcaataactcaaattctctgtctatgacaactgctgtatgactatatgaagaaatgggataaagatgctatagtcactcgaggacagataaaggcctgctcacagagcaagaatggaaaggagaggaaggcaggtcatgggcttcagttcagagaaggaagcacagcaaatacaaggtgagggccatgctagttactcccttgttcatcatgtggacagctagagaagcccactgacatgggaagtctgggctaactgactacattgagaggaggaccaggaagcagggcatgagagagaacatcagaccatgggagggatctgtgtagatgccagggtcctaaagagagtctttaaataaatgtgtggggtagcaccttggtgatggtccctttctgagttgggattctagtgggtagctgcaggatacagagagcagaaatcatctaatgggaaaccactgtagagatgataggactcgtcaccaaattccagatatattgtagtcattcaccatg

SEQ ID NO: 14 - DNA, 40 bp, synthetic construct; identical to SEQ ID NO: 11
  source 1..40: mol_type = other DNA

SEQ ID NO: 15 - DNA, 50 bp, synthetic construct
  source 1..50: mol_type = other DNA
ttctgcatggtccagggattgatcagacagatagtgaagttctgaggaca

SEQ ID NO: 16 - DNA, 25 bp, synthetic construct
  source 1..25: mol_type = other DNA
taggaagtggctcagaagtctgcct

SEQ ID NO: 17 - DNA, 25 bp, synthetic construct
  source 1..25: mol_type = other DNA
tctccagccccatttatcccagcta

SEQ ID NO: 18 - DNA, 25 bp, synthetic construct
  source 1..25: mol_type = other DNA
tgggatagctatgtggggagaccag

SEQ ID NO: 19 - DNA, 25 bp, synthetic construct
  source 1..25: mol_type = other DNA
agttctgtcatcaggccaactacgc

SEQ ID NO: 20 - DNA, 24 bp, synthetic construct
  source 1..24: mol_type = other DNA
tccttgcccctcagtcagtcttga

SEQ ID NO: 21 - DNA, 23 bp, synthetic construct
  source 1..23: mol_type = other DNA
tcctccatggcttggtgccttct

SEQ ID NO: 22 - DNA, 23 bp, synthetic construct
  source 1..23: mol_type = other DNA
gccagctcttgaggacctgctcc

SEQ ID NO: 23 - DNA, 25 bp, synthetic construct
  source 1..25: mol_type = other DNA
tggctcagacaaggagagcaagacc

SEQ ID NO: 24 - DNA, 25 bp, synthetic construct
  source 1..25: mol_type = other DNA
attagcctgcctaaaccaaccaggc

SEQ ID NO: 25 - DNA, 25 bp, synthetic construct
  source 1..25: mol_type = other DNA
tcaatacctgggccaccctctaaca

SEQ ID NO: 26 - DNA, 24 bp, synthetic construct
  source 1..24: mol_type = other DNA
gaagtctgcctgaccctctacatg

SEQ ID NO: 27 - DNA, 26 bp, synthetic construct
  source 1..26: mol_type = other DNA
cacagatgtgtaatacatgtacttgc

SEQ ID NO: 28 - DNA, 25 bp, synthetic construct
  source 1..25: mol_type = other DNA
tgtgtgctgatacagaagcctgagc

SEQ ID NO: 29 - DNA, 26 bp, synthetic construct
  source 1..26: mol_type = other DNA
tcccatcaccctgtgttatcatctgt

SEQ ID NO: 30 - DNA, 25 bp, synthetic construct
  source 1..25: mol_type = other DNA
accatatctagccctgcctgcctta

SEQ ID NO: 31 - DNA, 25 bp, synthetic construct
  source 1..25: mol_type = other DNA
tcaatacctgggccaccctctaaca

SEQ ID NO: 32 - DNA, 21 bp, synthetic construct
  source 1..21: mol_type = other DNA
acttgccccctgtcctcagtg

SEQ ID NO: 33 - DNA, 20 bp, synthetic construct
  source 1..20: mol_type = other DNA
gtggctagaaggcagctcca

SEQ ID NO: 34 - DNA, 20 bp, synthetic construct
  source 1..20: mol_type = other DNA
cagagaagccatcccgtggt

SEQ ID NO: 35 - DNA, 25 bp, synthetic construct
  source 1..25: mol_type = other DNA
taccatatctagccctgcctgcctt

SEQ ID NO: 36 - DNA, 25 bp, synthetic construct
  source 1..25: mol_type = other DNA
ttcgtggcctcgcagatgagtttag

SEQ ID NO: 37 - DNA, 25 bp, synthetic construct
  source 1..25: mol_type = other DNA
cagacctcacctttgtccccagttc

SEQ ID NO: 38 - DNA, 25 bp, synthetic construct
  source 1..25: mol_type = other DNA
ggtactgctatggaagggaccatgc

SEQ ID NO: 39 - DNA, 25 bp, synthetic construct
  source 1..25: mol_type = other DNA
gcctctttgggctcatatcctggac

SEQ ID NO: 40 - DNA, 25 bp, synthetic construct
  source 1..25: mol_type = other DNA
gagtgtgtcacatgactccactggg

SEQ ID NO: 41 - DNA, 8505 bp, synthetic construct
  source 1..8505: mol_type = other DNA
tcaatccctggaccatgcagaagggccttgtgggacttctcatacccacactcagctaatccccaaagattgactgaagcaagacatccccccaactttgatccctccccttccttgctcttcccccattcattccctcccttttctcagctgcttcatcaacacaacacttgacatccacacctcagaaaccctgtactaacagtgcaaggatgcagctgggtgagcaaaggcactagctatgcatggtcccttccatagcagtaccattccatcctgtggccctcctctctcactgcgttccatcaccctgggctccacggaatactctgatctctcaccatcactttttgtccatcttctttaactccagagtcatgggattcccagtcctctaagagagtcactccaactctccaagcaaagaatcactccacagaagccaccaaagctattaccaccaaaaaggacatagaaggtaagaagtccaatgtgccaactcatctcttcaaccttacacctcttgctcctggaaaccctccatgactctttctctgagctgtcaatgtacatcctaggtagaacaggttactaaaatgacttctgtactactgcaagccaggccttatatccaggcacaacaaataccaccacctaaccctgagaagccatcatccctttttgagctaatgctaaacagaggaaggacaaggacctacttagcccataggacctgagctctgtgtatctgatccaaactgtgaggtcctcatggattctgtaggcatgtgaccagacaaacaacaagacatttctgaggtgcataagaatctcattaaagggctagagaccattacacctagattggaagacttattgtaccccacatgttgtcttcacctcttcctaactattctctctctctctctctctctctctctctctctctctctctctctctctctctctctctctctctcacacacacacacacacacacacacacacagagcctaactaggttggaagataggaagcctaggattagcctggccttcagaatacagaatagggacatagagaatagctggggcaccaaagaaacctacaaaaagtcacacagaagcaagcatgggtctgttcaggcagcactgggcatctctaggagctagacagtgaccaggagaagcaatgtctccaacataggcctttccttaccaagcccttgggccccaaggcaagggcagcctattttctagatgaaacctatcccaccccaccccaaggctgtccaatgccccacctaaccatatgtccttcccccaacattgttcaagccataactgtaatcagataagaaaggggatttcctctgtcctctcacacatttctagatacctccatcccaaacatgacaagattagcctcttgccacttctccttgcaggggccatggcacccagcaacctcactgtgaacatcctgaccacatccacccatcctgagatgtcatcttggctcctgtgtgaagtatctggcttcttccccgaaaatatccacctcatgtggctgagtgtccacagtaaaatgaagtctacaaactttgtcactgcaaaccccaccccccagcctgggggcacattccagacctggagtgtcctgagactaccagtcgctctgagctcatcacttgacacttacacatgtgtggtggaacatgaggcctcaaagacaaagcttaatgccagcaagagcctagcaattagtggtaagtcacaactgggtaagagtgtcaagcaaggacagcacttggtacctatgatagacaaatactcctgtttgggaagaggaggtctgcattgtctagataagaggaacactgtgcttatctgtttcagtttaaaagacagaactacataacacttcaccctttctacaacacttagatttttcagccctctcctctatggtgtttcctgaaccctgaagaaagtgatatagatgtttatttattttctagggccactgttccaaagaaacaatcacgtattattagcagcttgaccaggtgtgaatttcccccagtatccacttctgctccctgtgggatgaagtccctccataagtaaagttgaaggctgtgggcataaatgcaaatgcctgtaatccaattttacaagtccatgacatacatttaacaaacaatagcagttacttctctactggagcctatgaactccccagctataggtttttcccttctgcagagcagagcttaaatccaatagggaagaagttggttgtacccataactgccataccactattacaccaataagcacatcttacatagcaagacaattagtgtagcactcaaagtccatgttcaagagagaatgttggcaacagcctatataacatctcccagcacagaaaagaaaaggaaaggaaaggaaaggaaaggaaaggaaaggaaaggaaaggaaaggaaaggaaaggaaaggaaaggaaaggaaaggaaagaaaagaaaagaaaagaaaagaaaagaaaagaaaagaaaagaaaagaaaagaaaagaaaagaaaagaaaagaaaagaaaagaaaggaaaagaaaggaaaagaaaggaagacagccagaaggcagcttccacttcagtcctagcccatgttttctatgtcttacagccaaggttagtgaagtctctagcagtaggaatttatcatctacttctggcatgcaagtaaaatcaatggtgataacttttgtggtttgtggctccttgggagcctccctgaacaataatttatgcagaggtagcccagctttgccactggggcttccattaaacaaactttggattgagggagtgtctttttctacctgcacacagtgattcctatgttttaactcttaatgagtcactgttgtttgttgttgttgttattgttgttgttattgttgttgttgttgttgtttctatcaacccagacactatagggctataaggcaggtggaagcatcactggctccagctgttctcagtcatgctcttcctatcaatgtttgtgtaatgactgccccttttactgtcggttttctatgggtgcctatggataacgctgggttgggcgaaacccttgatggcagcttttcccactgcttgcttatccatgtaggtgagagcacattcactctcacttgagaggcttaggcctttatgaagtctgcctgacatatgggataggtaggcttatatgcttacccatcatgcacccatgtgctggtaccataagcaagatcagacctcacctgttcatgtagcatgcagaggatcagaatatttcacaagaatgccagaagctctcattactctctggccttctcttaaagatcacatcctctaacaaagcttaccaaagccactaacatctgagttagtcctactcaggtacacatttcttaatgctccagcaaaagcatctctacccacttcattgggcactaaacaccacttctcacatgtttagagttttttgttaagactttaaattatctttcagtcatgtctgatgttcatgggcacctcagccagtaataactgagtgtttctggtgtgtgcaggtttttgtctgttttgtgttgtttatatgccaggaaaaagctgtttagtacagatctaattggcattccccttccttcatggtgtggatatttcttttgagttaaaacatcccctcttgtggcagggggagggctagaggtcaatggaatgatttcccgagtttccttggataaatgaaacctttgtctcagggaaggaagaatggttctacttctgtttctccacttcatgatgctgactctagcctcttccatgccccgtgtctccaggctatgaaggaatccagcatgcttttctctcttccttccttatcttcttcatgggttccaagtgggttcctgtggtttccacaggaaaggctttttagcacaaatctgatctgtactgtctttcctttatcccctgccagctctgagacagaaagccagcatctctatcagaatctctcttttgccttcagaatcagagaccactgtgaaaggcagcatccgtgcatctttttttccattgtactccttcattacgtgagcaagatttttctaagcagatagcttagccttcatcgcacatcttagcacacactgcaaatgtcacacagacagcttgctatgccatcccatgtatactattttcaaggattgcaataccttctctgatctgaaatccattcaagtaaaacaacatgttgcaggtccaattcaagaggcatgccacctcataccatataaaagaagatggtcagcaaaaattcctgctctctcagacaactcagacttgggagcagaaattcctatcacctctagcagggagtaaacaactactactacagctggaagcatactatctcctaccataaaagatgctagcttctctgacaccttagacctgaggcaaggaatcctctgcctcatttctttcgtctgctggaaagcctacaggactaagggctgctgtaagcttgacacaagcttgaggttgaaaggtgtcagacaggcttcacctgaggggccctatatatgagtgtagtgaggatggaacaggctacgtggggcatcctccggcatcttgtcctcagagtggccttcagagcccaggctcaagtcagccatacttccccacttagactctggaaatgccacagtatgtccacaacctccacacccagctacacaaaattgagagagggacctagtgggactgcagcaatcacatgaggccatacctggaccagccaagctgccagtataacagtcgtggctctgagattttgaaagcttggtctgttgtctatgcctcgggatctttgggatgccttaggatccatcgagaaagttctagagatcatgcctgtttctggctgcactgtactgactgagaacaggggctacagcaccactgtttctcatggcctacttcctgccaggtggtgcctcattagtactgaaaggagcctgacctacacttgccccaaattggccttttcttttcccctgtggcattggttcctcaaaggacactaaggttttctcttacttgactccctcaggatctctctacagattttcacaggtcactcctcagagctagagtcataccagcatgactatcctctgcccacattcacacagcatgcttaggaagtctgggcatgctctgtagtcatcttcattaggttgttcccacctgcctctctatctgcccctgccccacttccccttgttctgcctgttggtctctccctactctgtgcttcacatgtcttgaggtagatacatctgcctgagtcccacataacacttcttcatcacaccaacccctgagaacaataatgttcttagttctgtcgactagcacctcctgacttgtctctatgatgccattcttcccctcctacccatccatcaccctgttgccaaggcactctctgagacatcttgtttctacatagatacagaagaacatttgattaggcatgtaggccatggtgaaagaaaaacaccaagtaaacttttaacatcacaattttatttaaaactactagagaggactcagcaatgctcaaagagactaaattcccaggtgaaaggagatgggatgttgtattctaggcacagacagacagagcagtgggacatatgtaatcaccaagaaatcagccaaatttttaataaacaccttagctgccgagagggatgagaacacagaagaatcccaaattctgagccaatcaccactcacttaacctcccacccaggacactgctaagcaagaaacaccttcgagcacagtgtgggcagaatatccatcttgtaccagctccaaggcctgctgaatataagggttatcccaaagcacctagagccagaagtcaactatgataccccagctccagcacagctcagttcccagttccaaaaaacaggtcttccatgcctggtggataagatgatatgcacttgtatctcacccccagactctacaatccatgtatatacaatgcctagcatgcaatcagtaattacaagatacaaatgattgataaccaagacagagacagtacatgggattagacctgaggtacgggaaaggttgggaaagcattcatagtgtcccttgctacaagaagtcccagagctttgcagggtgtgagcagattctaacctcccccattggcaagtcttctcaaagctgcttctgctactagagaaccctctttcccagccacctgccactatttatcacttatctgagttttccccatgattctaggatgctaccacctcctgcctgagtcagacggtccttccaggagacctgatggtcctgcccttgcctgagacctttctaggctgaatggtcatcatgtccactgtctggttagcttggttgcctctgtgttaagttgcttcatactacatgtagcaaggaaagttacctttccacttctcaggacactgtaaagaagcacttctgtaaattatagtgagaaacgatcaataataaatgaaatgaacaaacataaacctctcttatctctatgggacctgccctaaggcctcttgtggatgcctgaaaccttgagtgttcttaagccatgtgtcacttcagtcattaacctgtggctgaggtggatcctgactgacaaacagacaggcatgtagacagcttgaagcctggacaaagagaaggttgacgttccaggcaaagcaagagcctatcacattgttccaaagtgtgtgatttagaatcagaagatttcttgtttaaaatatactcaaaaagttataaaatatcagccaggactagtcataaagcttaaatctgagagagagagagagagagagagagagagagagaggtgttcatgtatatacatatgtgtgtgtgtgtatatatatgtatgtatatgtatgtatatgtgtatattggtgtttgggtgtcagattccctggaattaaagttataaacagttatgagctgcaaagtaggctttccaatgtcttgttctccaaccataggtaattgaaactggaaactacaggtaaaggaggctgccaagtgtttcttattatgctaagattcagtgttctacaatattcacatgatttcactttgatatggtgaaatgcctttaggccaacgcatccacagacttcacagtgaccccagctgagcttacatataatgtcaaacagcacttgttgaatagatacccaggcagccctgtgacccatggtgttttccttaattcttcccatctgccttggactttcacaccttgagttgagatgtgctttctgtagaaatgtatttccctaggtctgtacatcagccaccaccctttctgatcttggaccatacttcagtctccagctgggatattcatatgcatgcttagatatggcataaccatcgttaacgtcttctgttacctttaggattaagttaccgtagctaggatgctttccaatctctccctccatttgcaaaggctcagggttccttctgactctggagtagtactctttcccaatgagtgcaccatgaccactattcttccttagaatgttactcccctttctctgcctttttcatggcacatctttctgagaaaacctacctggtctagctgctctttctgggtcttcttaagtagtaacctaactcctgcaccatccgtgtggcagtgacatagatccacatcaggtttggattcagatattccaatcccatgcagaccaatctatgtgtgagattccaggatccagcaggctggggcatgggttgaggggtaggattctttggggcaaagtggttagtaggcacttgggactggaagtgataagtattagcgggaggagggcatatgatcaaaacattgctgctggatgtgaaagctgtaccttcaggttcagtgttcaggagaccaggttaataagaaatgatctttctgtagggtggagaccttctcatgagcactagttcttccctaggcatagtcaacaccatccaacactcgtgtatcatggatgagcaaagtgacagctacatggacttagaggaggagaacggcctgtggcccacaatgtgcaccttcgtggccctcttcctgctcacactgctctacagtggcttcgtcaccttcatcaaggtaggcctcctaccacctccttctgaacccttcctgggcacatgtaagatacccttccaggggcattttgaaacccatcggtacttctggagtggctagggaatgcacaagtaattgggaggccatggtcactgttcagcttctgggccctatgtagctccacggactttccctgtttctcagacaacagcctctgtatgacttcacggctctctggcaggtgaagtag

SEQ ID NO: 42 - protein, 5 aa, synthetic construct
NAWMN

SEQ ID NO: 43 - protein, 19 aa, synthetic construct
HIKSKTDGGTTDYAAPVKG

SEQ ID NO: 44 - protein, 19 aa, synthetic construct
DRAYCGGDCSHYYYHDLDV

SEQ ID NO: 45 - protein, 5 aa, synthetic construct
SYAMS

SEQ ID NO: 46 - protein, 17 aa, synthetic construct
EISGSGSSTNYADSVKG

SEQ ID NO: 47 - protein, 10 aa, synthetic construct
DDSSGWAFNI

SEQ ID NO: 48 - protein, 5 aa, synthetic construct
SYGMY

SEQ ID NO: 49 - protein, 17 aa, synthetic construct
FIRFDESKKYYADSVKG

SEQ ID NO: 50 - protein, 17 aa, synthetic construct
DGEGGYCTNGVCHGLDV

SEQ ID NO: 51 - protein, 5 aa, synthetic construct
SFAMS

SEQ ID NO: 52 - protein, 17 aa, synthetic construct
EISDSGGNTYYADSVKG

SEQ ID NO: 53 - protein, 10 aa, synthetic construct
DDSSGWAFNI

SEQ ID NO: 54 - protein, 8 aa, synthetic construct
GFPFSNAW

SEQ ID NO: 55 - protein, 10 aa, synthetic construct
IKSKTDGGTT

SEQ ID NO: 56 - protein, 21 aa, synthetic construct
TTDRAYCGGDCSHYYYHDLDV

SEQ ID NO: 57 - protein, 8 aa, synthetic construct
GFTFSSYA

SEQ ID NO: 58 - protein, 8 aa, synthetic construct
ISGSGSST

SEQ ID NO: 59 - protein, 12 aa, synthetic construct
AKDDSSGWAFNI

SEQ ID NO: 60 - protein, 8 aa, synthetic construct
GFTLSSYG

SEQ ID NO: 61 - protein, 8 aa, synthetic construct
IRFDESKK

SEQ ID NO: 62 - protein, 19 aa, synthetic construct
ARDGEGGYCTNGVCHGLDV

SEQ ID NO: 63 - protein, 8 aa, synthetic construct
GFTFSSFA

SEQ ID NO: 64 - protein, 8 aa, synthetic construct
ISDSGGNT

SEQ ID NO: 65 - protein, 12 aa, synthetic construct
AKDDSSGWAFNI

SEQ ID NO: 66 - protein, 130 aa, synthetic construct
QVTLKESGGGLVKPGGSLRLSCAASGFPFSNAWMNWVRQAPGKGLEWVGHIKSKTDGGTTDYAAPVKGRFTISRDDSKNMVFLQMNSLKTEDTAVYYCTTDRAYCGGDCSHYYYHDLDVWGQGTTVTVSS

SEQ ID NO: 67 - protein, 119 aa, synthetic construct
EVQLVESGGGLVQPGGSLRLTCAASGFTFSSYAMSWVRQAPGKGLERVSEISGSGSSTNYADSVKGRFTISRDNSKNTLYLQMNSLRAEDTAVYYCAKDDSSGWAFNIWDQGTVVTVSS

SEQ ID NO: 68 - protein, 126 aa, synthetic construct
EVQLVQSGGGVVQPGRSLRLSCAASGFTLSSYGMYWVRQAPGKGREWVAFIRFDESKKYYADSVKGRFTISRDNSKNTLYLQMNSLRAEDTAVYYCARDGEGGYCTNGVCHGLDVWGQGTTVTVSS

SEQ ID NO: 69 - protein, 119 aa, synthetic construct
QLQLQESGGGLVQPGGSLRLTCAASGFTFSSFAMSWVRQAPGKGLERVSEISDSGGNTYYADSVKGRFTISRDNSKNTLYLQMNSLRAEDTAVYYCAKDDSSGWAFNIWDQGTVVTVSS

SEQ ID NO: 70 - protein, 760 aa, synthetic construct
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 71 - protein, 727 aa, synthetic construct
MERVQPLEENVGNAARPRFERNKLLLVASVIQGLGLLLCFTYICLHFSALGGGGSCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 72 - protein, 118 aa, synthetic construct
EVQLVQSGAEVKKPGESLKISCKGSGYSFTNYWLGWVRQMPGKGLEWMGDIYPGGDYPTYSEKFKVQVTISADKSISTAYLQWSSLKASDTAMYYCARSGNYDEVAYWGQGTLVTVSS

SEQ ID NO: 73 - protein, 112 aa, synthetic construct
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQSTHVPWTFGQGTKVEIK

SEQ ID NO: 74 - protein, 760 aa, synthetic construct
MMDQARSAFSNLFGGEPLSYTRFSLARQVDGDNSHVEMKLAADEEENADNNMKASVRKPKRFNGRLCFAAIALVIFFLIGFMSGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 75 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0078197 A1
(43) Pub. Date: Mar. 19, 2026

(54) GENETICALLY MODIFIED NON-HUMAN ANIMALS AND METHODS FOR PRODUCING HEAVY-CHAIN ANTIBODIES

(71) Applicant: Biocytogen Pharmaceuticals (Beijing) Co., Ltd., Beijing (CN)

(72) Inventors: Yiqing Hu, Beijing (CN); Qi Zhang, Beijing (CN); Lijun Zhang, Beijing (CN); Yabo Zhang, Beijing (CN); Huizhen Zhao, Beijing (CN); Jiawei Yao, Beijing (CN); Yuelei Shen, Beijing (CN)

(21) Appl. No.: 19/108,869

(22) PCT Filed: Sep. 15, 2023
(86) PCT No.: PCT/CN2023/118958

§ 371 Date: Mar. 5, 2025

(30) Foreign Application Priority Data
Sep. 16, 2022 (WO) PCT/CN2022/119188
Dec. 2, 2022 (WO) PCT/CN2022/136246

(51) Int. Cl. C07K 16/28; A01K 67/0276; A01K 67/0278; C12N 15/85

(52) U.S. Cl. CPC C07K 16/2881; A01K 67/0276; A01K 67/0278; C12N 15/8509; A01K 2217/072; A01K 2217/075; A01K 2227/105; A01K 2267/01; C07K 2317/24; C07K 2317/33; C07K 2317/92; C07K 2317/94; C12N 2015/8518

(57) ABSTRACT

The present disclosure relates to genetically modified animals and methods for producing heavy-chain antibodies. In one aspect, the genetically modified non-human animal comprises a modified immunoglobulin heavy chain locus, wherein the modified immunoglobulin heavy chain locus comprises an IgG constant region gene, wherein the IgG constant region gene encodes an IgG heavy chain constant region lacking a CH1 domain, wherein the genetically modified non-human animal expresses a heavy-chain antibody. Also disclosed herein are anti-TFR1 antibodies, antigen-binding fragments, and the uses thereof.

### CLAIM OF PRIORITY

This application claims priority to PCT/CN2022/119188, filed on Sep. 16, 2022 and PCT/CN2022/136246, filed on Dec. 2, 2022. The entire contents of the foregoing are incorporated herein by reference.

### TECHNICAL FIELD

This disclosure relates to genetically modified animals and methods for producing heavy-chain antibodies. The disclosure also relates to anti-TFR1 antibodies, antigen-binding fragments, and the uses thereof.

### BACKGROUND

Therapeutic antibodies are one of the fastest growing classes of therapeutic compounds, rapidly outpacing the growth of small-molecule drugs. For example, monoclonal antibodies have revolutionized cancer therapy. However, delivery to tumor cells in vivo is hampered by the large size of conventional antibodies. The minimal target recognition module of a conventional antibody is composed of two non-covalently associated variable domains (VH and VL). The inherent hydrophobic interaction of VH and VL domains limits the stability and solubility of engineered antibodies, often causing aggregation and/or mispairing of V-domains.

The discovery of heavy-chain antibodies has given rise to unprecedented opportunities in impacting cancer therapy. These unique form of camelid-derived antibodies lack the entire light chain and the CH1 domain and are only composed of a single variable domain termed VHH. Recombinant VHHs are small (15-20 kDa) and strictly monomeric; they bind their target with nM affinity as well as with being stable in a broad pH and temperature ranges. Molecular manipulation is also easier with VHH; this facilitates the production of multivalent formats of monoclonal antibodies compared with conventional recombinant antibodies and their fragments, which is problematic due to aggregation and reduced affinity. Moreover, VHH often binds to epitopes that are less immunogenic for conventional antibodies.

Usually, the therapeutic antibodies are human or humanized antibodies. The human or humanized antibodies can be generated by humanization of a rodent antibody (e.g., a mouse antibody) or by using phage libraries. However, these animals or phage libraries usually cannot produce heavy chain antibodies. Instead, heavy chain antibodies are often obtained from camelid heavy chain antibodies. These camelid heavy chain antibodies need to be humanized. The humanization process may adversely affect the binding affinity and introduce immunogenic epitopes to the antibodies. Iterative and time-consuming experiments are often required to improve the properties of these antibodies. And in some cases, these antibodies can also be immunogenic in patients, leading to attenuation of their efficacy over time. Therefore, there is a need for an efficient and reliable platform to produce human or humanized heavy chain antibodies and nanobodies.

### SUMMARY

The present disclosure relates to genetically-modified animals and cells with humanized immunoglobulin heavy chain variable region locus and truncated immunoglobulin heavy chain constant region locus. For example, the CH1 coding region within the IGHG1 gene can be knocked out such that the expressed IgG does not include the CH1 domain. In some embodiments, the immunoglobulin light chain (e.g., kappa and lambda) loci are also knocked out. Upon immunization, the animals can produce heavy-chain antibodies with high affinity/diversity. In some embodiments, the heavy-chain antibodies can be further processed to generate nanobodies.

In one aspect, the disclosure is related to a genetically modified non-human animal comprising a modified immunoglobulin heavy chain locus, in some embodiments, the modified immunoglobulin heavy chain locus comprises an IgG constant region gene, in some embodiments, the IgG constant region gene encodes an IgG heavy chain constant region lacking a CH1 domain, in some embodiments, the genetically modified non-human animal expresses a heavy-chain antibody. In some embodiments, the animal comprises exactly one IgG constant region gene. In some embodiments, the IgG heavy chain constant region gene is IGHG1. In some embodiments, the IgG heavy chain constant region comprises or consists of a CH2 domain and a CH3 domain, and optionally a hinge region.

In one aspect, the disclosure is related to a genetically modified non-human animal whose genome comprises a germline genetic modification comprising a deletion of IGHG3, IGHG2b, and IGHG2c genes and a deletion of the CH1 exon of IGHG1 gene at an endogenous immunoglobulin heavy chain gene locus. In some embodiments, the germline genetic modification further comprises a deletion of endogenous IGHE gene at the endogenous immunoglobulin heavy chain gene locus. In some embodiments, the genetic modification further comprises a deletion of endogenous Sγ2b, Sγ2c, and Sa switch regions at the endogenous immunoglobulin heavy chain gene locus. In some embodiments, the modified immunoglobulin heavy chain gene locus comprises a modified IGHG1 gene lacking a sequence encoding a CH1 domain, in some embodiments, the modified IGHG1 gene comprises a sequence that is at least 80%, 90%, 95% or 99% identical to SEQ ID NO: 1. In some embodiments, the genetic modification further comprises a deletion of endogenous Sγ3 switch region at the endogenous immunoglobulin heavy chain gene locus. In some embodiments, the animal's genome comprises endogenous Sμ, Sγ1, Sα switch regions, the modified IGHG1 gene lacking a sequence encoding a CH1 domain, and endogenous IGHM, IGHδ, IGHA genes. In some embodiments, the genetic modification further comprises a deletion of endogenous IGHM and IGHδ genes at the endogenous immunoglobulin heavy chain gene locus. In some embodiments, the animal genome comprises endogenous Sμ, Sγ1, Sα switch regions, the modified IGHG1 gene lacking a sequence encoding a CH1 domain, and the endogenous IGHA gene. In some embodiments, the Su and Syl switch regions are linked with a sequence that is at least 80%, 90%, 95% or 99% identical to SEQ ID NO: 8. In some embodiments, the genetic modification further comprises a deletion of the CH1 coding sequence of IGHM gene at the endogenous immunoglobulin heavy chain gene locus. In some embodiments, the animal's genome comprises endogenous Sμ, Syl, Sα switch regions, a modified IGHM gene lacking a sequence encoding a CH1 domain, the modified IGHG1 gene lacking a sequence encoding a CH1 domain, and endogenous IGHδ, IGHA genes.

In some embodiments, the Su switch region and the modified IGHM gene are linked with a sequence that is at least 80%, 90%, 95% or 99% identical to SEQ ID NO: 10. In some embodiments, the genetic modification further comprises a deletion of the CH1 exon of IGHM gene and a deletion of IGHδ gene at the endogenous immunoglobulin heavy chain gene locus. In some embodiments, the animal's genome comprises endogenous Sμ, Sγ1, Sα switch regions, a modified IGHM gene lacking a sequence encoding a CH1 domain, the modified IGHG1 gene lacking a sequence encoding a CH1 domain, and an endogenous IGHA gene. In some embodiments, the genetic modification further comprises a deletion of the CH1 exon of IGHM gene and a deletion of the CH1 coding sequence of IGHδ gene at the endogenous immunoglobulin heavy chain gene locus. In some embodiments, the animal's genome comprises endogenous Sμ, Sγ1, Sα switch regions, a modified IGHM gene lacking a sequence encoding a CH1 domain, a modified IGHδ gene lacking a sequence encoding a CH1 domain, the modified IGHG1 gene lacking a sequence encoding a CH1 domain, and an endogenous IGHA gene. In some embodiments, the modified IGHM gene are linked with a sequence that is at least 80%, 90%, 95% or 99% identical to SEQ ID NO: 10, and the modified IGHδ gene comprises a sequence that is at least 80%, 90%, 95%, or 99% identical to SEQ ID NO: 41. In some embodiments, the modified IGHM gene comprises a sequence that is at least 80%, 90%, 95%, or 99% identical to SEQ ID NO: 13. In some embodiments, the genetic modification further comprises a deletion of endogenous Sγ1 switch region at the endogenous immunoglobulin heavy chain gene locus. In some embodiments, the animal's genome comprises endogenous Sμ, Sγ3, Sα switch regions, the modified IGHG1 gene lacking a sequence encoding a CH1 domain, and endogenous IGHM, IGHδ, IGHA genes. In some embodiments, the genetic modification further comprises a deletion of endogenous Sγ3 switch region at the endogenous immunoglobulin heavy chain gene locus. In some embodiments, the genetic modification further comprises a deletion of endogenous IGHM and IGHδ genes at the endogenous immunoglobulin heavy chain gene locus. In some embodiments, the animal's genome comprises endogenous Sμ, Sα switch regions, the modified IGHG1 gene lacking a sequence encoding a CH1 domain, and the endogenous IGHA gene. In some embodiments, the Su switch region and the modified IGHG1 gene are linked with a sequence that is at least 80%, 90%, 95%, or 99% identical to SEQ ID NO: 9. In some embodiments, the modified genome comprises a functional IGHM gene.

In some embodiments, an animal can still have the endogenous sequence when the sequence in the genome is replaced by the same sequence or the sequence from the same animal.

In one aspect, the disclosure is related to a genetically-modified non-human animal whose genome comprises the following elements, at an endogenous immunoglobulin heavy chain gene locus in a 5′ to 3′ order: Su switch region, IGHM gene, IGHδ gene, Sγ1 switch region, IGHG1 gene lacking a sequence encoding a CH1 domain, Sα switch region, and IGHA gene. In some embodiments, the elements are operably linked. In some embodiments, the endogenous immunoglobulin heavy chain constant region gene locus consists of the following functional genes and switch regions: Su switch region, IGHM gene, IGHδ gene, Sγ1 switch region, IGHG1 gene lacking a sequence encoding a CH1 domain, Sα switch region, and IGHA gene.

In one aspect, the disclosure is related to a genetically-modified non-human animal whose genome comprises the following elements, at an endogenous immunoglobulin heavy chain gene locus in a 5′ to 3′ order: Su switch region, IGHM gene, IGHδ gene, Sγ3 switch region, IGHG1 gene lacking a sequence encoding a CH1 domain, Sα switch region, and IGHA gene. In some embodiments, the elements are operably linked. In some embodiments, the endogenous immunoglobulin heavy chain constant region gene locus consists of the following functional genes and switch regions: Su switch region, IGHM gene, IGHδ gene, Sγ3 switch region, IGHG1 gene lacking a sequence encoding a CH1 domain, Sα switch region, and IGHA gene.

In one aspect, the disclosure is related to a genetically-modified non-human animal whose genome comprises the following elements, at an endogenous immunoglobulin heavy chain gene locus in a 5′ to 3′ order: Su switch region, Sγ1 switch region, IGHG1 gene lacking a sequence encoding a CH1 domain, Sα switch region, and IGHA gene. In some embodiments, the elements are operably linked. In some embodiments, the endogenous immunoglobulin heavy chain constant region gene locus consists of the following functional genes and switch regions: Su switch region, Sγ1 switch region, IGHG1 gene lacking a sequence encoding a CH1 domain, Sα switch region, and IGHA gene.

In one aspect, the disclosure is related to a genetically-modified non-human animal whose genome comprises the following elements, at an endogenous immunoglobulin heavy chain gene locus in a 5′ to 3′ order: Su switch region, IGHG1 gene lacking a sequence encoding a CH1 domain, Sα switch region, and IGHA gene. In some embodiments, the elements are operably linked. In some embodiments, the endogenous immunoglobulin heavy chain constant region gene locus consists of the following functional genes and switch regions: Su switch region, IGHG1 gene lacking a sequence encoding a CH1 domain, Sα switch region, and IGHA gene.

In one aspect, the disclosure is related to a genetically-modified non-human animal whose genome comprises the following elements, at an endogenous immunoglobulin heavy chain gene locus in a 5′ to 3′ order: Su switch region, IGHM gene lacking a sequence encoding a CH1 domain, IGHδ gene, Sγ1 switch region, IGHG1 gene lacking a sequence encoding a CH1 domain, Sα switch region, and IGHA gene. In some embodiments, the elements are operably linked. In some embodiments, the endogenous immunoglobulin heavy chain constant region gene locus consists of the following functional genes and switch regions: Su switch region, IGHM gene lacking a sequence encoding a CH1 domain, IGHδ gene, Sγ1 switch region, IGHG1 gene lacking a sequence encoding a CH1 domain, Sα switch region, and IGHA gene.

In one aspect, the disclosure is related to a genetically-modified non-human animal whose genome comprises the following elements, at an endogenous immunoglobulin heavy chain gene locus in a 5′ to 3′ order: Su switch region, IGHM gene lacking a sequence encoding a CH1 domain, Sγ1 switch region, IGHG1 gene lacking a sequence encoding a CH1 domain, Sα switch region, and IGHA gene. In some embodiments, the elements are operably linked. In some embodiments, the endogenous immunoglobulin heavy chain constant region gene locus consists of the following functional genes and switch regions: Su switch region, IGHM gene lacking a sequence encoding a CH1 domain, Sγ1 switch region, IGHG1 gene lacking a sequence encoding a CH1 domain, Sα switch region, and IGHA gene.

In one aspect, the disclosure is related to a genetically-modified non-human animal whose genome comprises the following elements, at an endogenous immunoglobulin heavy chain gene locus in a 5′ to 3′ order: Su switch region, IGHM gene lacking a sequence encoding a CH1 domain. IGHδ gene lacking a sequence encoding a CH1 domain, Sγ1 switch region, IGHG1 gene lacking a sequence encoding a CH1 domain, Sα switch region, and IGHA gene. In some embodiments, the elements are operably linked. In some embodiments, the endogenous immunoglobulin heavy chain constant region gene locus consists of the following functional genes and switch regions: Su switch region, IGHM gene lacking a sequence encoding a CH1 domain, IGHδ gene lacking a sequence encoding a CH1 domain, Sγ1 switch region, IGHG1 gene lacking a sequence encoding a CH1 domain. Sα switch region, and IGHA gene.

In some embodiments, the animal expresses a heavy-chain antibody comprising an IgG heavy chain constant region lacking the CH1 domain. In some embodiments, the heavy-chain antibody binds to it target antigen with a KD of less than 10⁻⁷ M, less than 10⁻⁸ M, or less than 10⁻⁹ M. In some embodiments, the heavy-chain antibody comprises or consists of a variable region, a CH2 domain and a CH3 domain. In some embodiments, the heavy-chain antibody further comprises a transmembrane domain and/or a cytoplasmic domain. In some embodiments, the genetically modified non-human animal does not express IgG antibodies comprising light chains. In some embodiments, the animal expresses IgM, IgD, and/or IgA (e.g., functional IgM, IgD, and/or IgA).

In some embodiments, the animal comprises at an endogenous immunoglobulin heavy chain gene locus, one or more human IGHV genes, one or more human IGHD genes, and one or more human IGHJ genes, in some embodiments, the human IGHV genes, the human IGHD genes, and the human IGHJ genes are operably linked and can undergo VDJ rearrangement. In some embodiments, the animal comprises at least 150 human IGHV genes selected from Table 1, at least 20 human IGHD genes selected from Table 2, and at least 5 human IGHJ genes selected from Table 3. In some embodiments, the animal comprises all human IGHV genes, all human IGHD genes, and all human IGHJ genes at the endogenous immunoglobulin heavy chain gene locus of human chromosome 14 of a human subject. In some embodiments, the animal comprises all human IGHV genes, all human IGHD genes, and all human IGHJ genes at the endogenous immunoglobulin heavy chain gene locus of human chromosome 14 of a human cell. In some embodiments, the animal is a mouse and the genetic modification in the animal's endogenous immunoglobulin heavy chain gene locus comprises a deletion of one or more mouse IGHV genes in Table 4, one or more mouse IGHD genes in Table 5, and/or one or more mouse IGHJ genes in Table 6. In some embodiments, the animal is a mouse and the genetic modification in the animal's endogenous heavy chain immunoglobulin gene locus comprises a deletion of a contiguous sequence starting from mouse IGHV1-85 gene to mouse IGHJ4 gene. In some embodiments, the animal comprises an unmodified human sequence derived from a human heavy chain immunoglobulin gene locus, in some embodiments, the unmodified human sequence is at least 800 kb. In some embodiments, the animal comprises an unmodified human sequence derived from a human heavy chain immunoglobulin gene locus starting from human IGHV(III)-82 to human IGHV1-2. In some embodiments, the animal comprises an unmodified human sequence derived from a human heavy chain immunoglobulin gene locus starting from human IGHV(III)-82 to human IGHV6-1. In some embodiments, the animal comprises an unmodified human sequence derived from a human heavy chain immunoglobulin gene locus starting from human IGHD1-1 to human IGHJ6. In some embodiments, the animal comprises an unmodified human sequence derived from a human heavy chain immunoglobulin gene locus starting from human IGHV(III)-82 to human IGHJ6.

In some embodiments, the genetically modified non-human animal described herein has a genome comprising, at the endogenous immunoglobulin heavy chain locus: a replacement of one or more endogenous IGHV, endogenous IGHD, and endogenous IGHJ genes with one or more human IGHV, human IGHD, and human IGHJ genes, in some embodiments, human IGHV, human IGHD, and human IGHJ genes are operably linked to one or more of endogenous IGHM, IGHδ, IGHG1 lacking a sequence encoding the CH1 domain, and IGHA genes. In some embodiments, one or more endogenous IGHV, endogenous IGHD, and endogenous IGHJ genes are replaced by at least 150 human IGHV genes in Table 1, at least 20 human IGHD genes in Table 2, and at least 5 human IGHJ genes in Table 3. In some embodiments, the animal is a mouse, and at least 180 mouse IGHV genes in Table 4, all mouse IGHD genes in Table 5, and all mouse IGHJ genes in Table 6 are replaced. In some embodiments, the animal is homozygous with respect to the immunoglobulin heavy chain gene locus. In some embodiments, the animal is heterozygous with respect to the immunoglobulin heavy chain gene locus. In some embodiments, the animal comprises an endogenous light chain immunoglobulin gene locus. In some embodiments, the animal comprises a disruption in the endogenous immunoglobulin light chain gene locus. In some embodiments, the animal lacks an endogenous immunoglobulin heavy chain variable region locus that is capable of rearranging and forming a nucleic acid sequence that encodes an endogenous heavy chain variable domain. In some embodiments, the animal can produce a humanized antibody. In some embodiments, the animal is a mammal. In some embodiments, the animal is a rodent. In some embodiments, the animal is a mouse. In some embodiments, the animal has substantially normal B cell development and maturation.

In one aspect, the disclosure is related to a cell obtained from the genetically modified non-human animal as described herein. In some embodiments, the cell is a B cell that expresses a chimeric immunoglobulin heavy chain comprising an immunoglobulin heavy chain variable domain that is derived from a rearrangement of one or more human IGHV genes, one or more human IGHD genes, and one or more human IGHJ genes, in some embodiments, the immunoglobulin heavy chain variable domain is operably linked to a non-human heavy chain constant region. In some embodiments, the cell is an embryonic stem (ES) cell.

In one aspect, the disclosure is related to a method of making an antibody that specifically binds to an antigen, the method comprising a) exposing the genetically modified non-human animal as described herein to the antigen; b) producing a hybridoma from a cell collected from the animal; and c) collecting a heavy-chain antibody produced by the hybridoma. In some embodiments, the method further comprises sequencing the genome of the hybridoma.

In one aspect, the disclosure is related to a method of making an antibody that specifically binds to an antigen, the method comprising a) exposing the genetically modified non-human animal as described herein to the antigen; b) sequencing nucleic acids encoding human immunoglobulin heavy chain variable regions in a cell that expresses a heavy-chain antibody that specifically binds to the antigen; and c) operably linking in a cell the nucleic acid encoding the human immunoglobulin heavy chain variable region with a nucleic acid encoding a human immunoglobulin heavy chain constant region.

In one aspect, the disclosure is related to a method of making an antibody that specifically binds to an antigen, the method comprising a) obtaining a nucleic acid sequence encoding human immunoglobulin heavy chain variable regions in a cell that expresses a heavy-chain antibody that specifically binds to the antigen, in some embodiments, the cell is obtained by exposing the genetically modified non-human animal as described herein to the antigen; b) operably linking the nucleic acid encoding the human immunoglobulin heavy chain variable region with a nucleic acid encoding a human immunoglobulin heavy chain constant region; and c) expressing the nucleic acid in a cell, thereby obtaining the antibody.

In one aspect, the disclosure is related to a method of obtaining a nucleic acid that encodes an antibody binding domain that specifically binds to an antigen, the method comprising a) exposing the genetically modified non-human animal as described herein to the antigen; and b) sequencing nucleic acids encoding human immunoglobulin heavy chain variable regions in a cell that expresses a heavy-chain antibody that specifically binds to the antigen.

In one aspect, the disclosure is related to a method of making an antibody that specifically binds to antigen, the method comprising a) exposing the genetically modified non-human animal as described herein to the antigen; b) constructing a phage plasmid library using RNA prepared from immune cells (e.g., splenocytes) of the animal; c) screening the phage plasmid library; and d) sequencing nucleic acids encoding human immunoglobulin heavy chain variable regions from a phage plasmid that encodes a heavy-chain antibody that specifically binds to the antigen. In some embodiments, screening comprises isolating phages expressing immunoglobulin heavy chain variable regions based on binding affinity to the antigen.

In one aspect, the disclosure is related to a method of obtaining a sample, the method comprising a) exposing the genetically modified non-human animal as described herein to the antigen; and b) collecting the sample from the animal. In some embodiments, the sample is an immune cell, a lymphoid tissue, a spleen tissue, a spleen cell, or a B cell.

In one aspect, the disclosure is related to an antibody or antigen-binding fragment thereof that binds to transferrin receptor 1 (TFR1), comprising: a heavy chain single variable domain (VHH) comprising complementarity determining regions (CDRs) 1, 2, and 3, in some embodiments, the VHH CDR1 region comprises an amino acid sequence that is at least 80%, 85%, 90%, 95%, 96%, 97%, 98%, 99%, or 100% identical to a selected VHH CDR1 amino acid sequence, the VHH CDR2 region comprises an amino acid sequence that is at least 80%, 85%, 90%, 95%, 96%, 97%, 98%, 99%, or 100% identical to a selected VHH CDR2 amino acid sequence, and the VHH CDR3 region comprises an amino acid sequence that is at least 80%, 85%, 90%, 95%, 96%, 97%, 98%, 99%, or 100% identical to a selected VHH CDR3 amino acid sequence; in some embodiments, the selected VHH CDRs 1, 2, and 3 amino acid sequences are one of the following:
(1) the selected VHH CDRs 1, 2, 3 amino acid sequences are set forth in SEQ ID NOs: 42, 43, and 44, respectively;
(2) the selected VHH CDRs 1, 2, 3 amino acid sequences are set forth in SEQ ID NOs: 45, 46, and 47, respectively;
(3) the selected VHH CDRs 1, 2, 3 amino acid sequences are set forth in SEQ ID NOs: 48, 49, and 50, respectively;
(4) the selected VHH CDRs 1, 2, 3 amino acid sequences are set forth in SEQ ID NOs: 51, 52, and 53, respectively;
(5) the selected VHH CDRs 1, 2, 3 amino acid sequences are set forth in SEQ ID NOs: 54, 55, and 56, respectively;
(6) the selected VHH CDRs 1, 2, 3 amino acid sequences are set forth in SEQ ID NOs: 57, 58, and 59, respectively;
(7) the selected VHH CDRs 1, 2, 3 amino acid sequences are set forth in SEQ ID NOs: 60, 61, and 62, respectively; and
(8) the selected VHH CDRs 1, 2, 3 amino acid sequences are set forth in SEQ ID NOs: 63, 64, and 65, respectively.

In some embodiments, the VHH comprises CDRs 1, 2, 3 with the amino acid sequences set forth in SEQ ID NOs: 42, 43, and 44, respectively. In some embodiments, the VHH comprises CDRs 1, 2, 3 with the amino acid sequences set forth in SEQ ID NOs: 45, 46, and 47, respectively. In some embodiments, the VHH comprises CDRs 1, 2, 3 with the amino acid sequences set forth in SEQ ID NOs: 48, 49, and 50, respectively. In some embodiments, the VHH comprises CDRs 1, 2, 3 with the amino acid sequences set forth in SEQ ID NOs: 51, 52, and 53, respectively.

In one aspect, the disclosure is related to an antibody or antigen-binding fragment thereof that binds to TFR1 comprising a heavy chain single variable region (VHH) comprising an amino acid sequence that is at least 80%, 85%, 90%, 95%, 96%, 97%, 98%, 99%, or 100% identical to a selected VHH sequence, in some embodiments, the selected VHH sequence is selected from the group consisting of SEQ ID NOs: 66, 67, 68, and 69. In some embodiments, the VHH comprises the sequence of SEQ ID NO: 66. In some embodiments, the VHH comprises the sequence of SEQ ID NO: 67. In some embodiments, the VHH comprises the sequence of SEQ ID NO: 68. In some embodiments, the VHH comprises the sequence of SEQ ID NO: 69. In some embodiments, the antibody or antigen-binding fragment specifically binds to a human TFR1, a monkey TFR1, a mouse TFR1, or a chimeric TFR1. In some embodiments, the antibody or antigen-binding fragment is a human or humanized antibody or antigen-binding fragment thereof. In some embodiments, the antibody or antigen-binding fragment is a multi-specific antibody (e.g., a bispecific antibody).

In one aspect, the disclosure is relate to an antibody or antigen-binding fragment thereof comprising the VHH CDRs 1, 2, 3, of the antibody or antigen-binding fragment thereof as described herein.

In some embodiments, the antibody or antigen-binding fragment comprises a human IgG Fc (e.g., a human IgG1 Fc). In some embodiments, the human IgG Fc comprises a non-asparagine residue (e.g., alanine) at position 297 according to EU numbering. In some embodiments, the antibody or antigen-binding fragment comprises two or more heavy chain single variable domains.

In one aspect, the disclosure is related to a nucleic acid comprising a polynucleotide encoding the antibody or antigen-binding fragment thereof as described herein. In some embodiments, the nucleic acid is cDNA.

In one aspect, the disclosure is related to a vector comprising one or more of the nucleic acids as described herein.

In one aspect, the disclosure is related to a cell comprising the vector as described herein. In some embodiments, the cell is a CHO cell. In one aspect, the disclosure is related to a cell comprising one or more of the nucleic acids described herein.

In one aspect, the disclosure is related to a method of producing an antibody or an antigen-binding fragment thereof, the method comprising (a) culturing the cell as described herein under conditions sufficient for the cell to produce the antibody or the antigen-binding fragment thereof; and (b) collecting the antibody or the antigen-binding fragment thereof produced by the cell.

In one aspect, the disclosure is related to an antibody-drug conjugate comprising the antibody or antigen-binding fragment thereof as described herein covalently bound to a therapeutic agent. In some embodiments, the therapeutic agent is a cytotoxic or cytostatic agent.

In one aspect, the disclosure is related to a method of treating a subject having a brain disease (e.g., a brain cancer), the method comprising administering a therapeutically effective amount of a composition comprising the antibody or antigen-binding fragment thereof, or the antibody-drug conjugate as described herein, to the subject. In some embodiments, the antibody or antigen-binding fragment thereof, or the antibody-drug conjugate can pass across the blood-brain barrier (BBB) of the subject.

In one aspect, the disclosure is related to a method of treating a subject having a cancer, the method comprising administering a therapeutically effective amount of a composition comprising the antibody or antigen-binding fragment thereof, or the antibody-drug conjugate as described herein, to the subject. In some embodiments, the cancer is brain cancer, lung cancer, gastric cancer, colorectal cancer, liver cancer, ovarian cancer, prostate cancer, leukemia, or breast cancer. In one aspect, the disclosure is related to a method of identifying a subject as having a brain disease (e.g., a brain cancer), the method comprising detecting a sample collected from the subject as having the brain disease by the antibody or antigen-binding fragment thereof as described herein, thereby identifying the subject as having the brain disease. In some embodiments, the sample is a brain parenchyma sample from the subject. In some embodiments, the subject described herein is a human subject.

In one aspect, the disclosure is related to a method of delivering an agent to cross blood brain barrier, the method comprising administering the agent covalently linked to the antibody or antigen-binding fragment thereof as described herein to the subject. In some embodiments, the agent is an antibody or an antibody drug conjugate. In some embodiments, the agent is anti-amyloid antibody.

In one aspect, the disclosure is related to a pharmaceutical composition comprising the antibody or antigen-binding fragment thereof as described herein, and a pharmaceutically acceptable carrier. In one aspect, the disclosure is related to a pharmaceutical composition comprising the antibody drug conjugate as described herein, and a pharmaceutically acceptable carrier.

In one aspect, the disclosure is related to an antibody or antigen-binding fragment thereof that cross-competes with the antibody or antigen-binding fragment thereof as described herein.

In one aspect, the disclosure provides a method of making an antibody that specifically binds to an antigen. The method involves exposing the animal as described herein to the antigen; obtaining the sequence of (e.g. by sequencing) nucleic acids encoding human heavy chain immunoglobulin variable regions in a cell that expresses a chimeric heavy chain antibody that specifically binds to the antigen; and operably linking in a cell the nucleic acid encoding the human heavy chain immunoglobulin variable region with a nucleic acid encoding a human heavy chain immunoglobulin constant region.

The disclosure also relates to an offspring of the non-human mammal. In some embodiments, the non-human mammal is a rodent. In some embodiments, the non-human mammal is a mouse.

The disclosure also provides to a cell including the targeting vector as described herein. The disclosure also relates to a cell (e.g., a stem cell, an embryonic stem cell, an immune cell, a B cell, a T cell, or a hybridoma) or a cell line, or a primary cell culture thereof derived from the non-human mammal or an offspring thereof. The disclosure further relates to the tissue, organ or a culture thereof derived from the non-human mammal or an offspring thereof.

The disclosure further relates to the use of the non-human mammal or an offspring thereof, the animal model generated through the method as described herein in the development of a product related to an immunization process, the manufacture of a human antibody, or the model system for research in pharmacology, immunology, microbiology and medicine.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram of heavy-chain antibody.

FIG. 2 shows schematic structures of IgM, IgD, IgG, IgE, and IgA immunoglobulin isotypes. Mouse and human immunoglobulin heavy chain locus genes are aligned and labelled with corresponding names.

FIG. 3 is a schematic diagram showing the constant region genes of mouse immunoglobulin heavy chain locus. The Cγ1 gene structure including exons CH1, H, CH2, CH3, M1, and M2 are shown.

FIGS. 4A-4B shows mutant alleles after genetic modifications at mouse immunoglobulin heavy chain constant region locus.

FIG. 5 shows gene structure of Cγ1ΔCH1. The CH1 coding region is either missing (left) or replaced by a Neo cassette (right).

FIG. 6 shows a workflow of genetic modifications using targeting vector V1 (Example 3). A 100883 bp sequence from Sγ3 to Cε at mouse immunoglobulin heavy chain constant region locus was replaced in a single step with a 16076 bp sequence including the mouse Sγ1 and the Cγ1ΔCH1 knock-in sequence to generate Mutant allele 1.

FIG. 7 shows a workflow of genetic modifications using targeting vector V2 (Example 3). A 92859 bp sequence from Cγ3 to Cε at mouse immunoglobulin heavy chain constant region locus was replaced in a single step with a sequence including the Cγ1ΔCH1 knock-in sequence to generate Mutant allele 1′.

FIG. 8 shows a workflow of genetic modifications using targeting vector V3 (Example 3). A 16434 bp sequence including Cμ and Cδ was knocked out from Mutant allele 1 to generate Mutant allele 2.

FIG. 9 shows a workflow of genetic modifications using targeting vector V4 (Example 3). A sequence including Cμ, Cδ, and Sγ1 was knocked out from Mutant allele 1 such that Su and Cγ1ΔCH1′ were directly connected, generating Mutant allele 2′.

FIG. 10 shows a workflow of genetic modifications using targeting vector V5 (Example 3). The CH1 coding sequence in Cμ was knocked out from Mutant allele 1 to generate Mutant allele 3.

FIG. 11 shows a workflow of genetic modifications using targeting vector V6 (Example 3). The CH1 coding sequence in Cμ and the entire Cδ were knocked out from Mutant allele 1 to generate Mutant allele 4.

FIG. 12 shows a workflow of genetic modifications using targeting vector V7 (Example 3). The CH1 coding sequence in Cμ and the CH1 coding sequence in Co were knocked out from Mutant allele 1 to generate Mutant allele 5.

FIGS. 13A-13B show PCR assay results using primer pairs L-GT-F1/L-GT-R1 and R-GT-F2/R-GT-R2, respectively, to verify the genotype of Mutant allele 1. WT is a wild-type control. H₂O is a blank control. M is a marker.

FIG. 14A shows Southern Blot results of Mutant allele 1 positive clones digested with Bell and hybridized with LR probe. M is a marker. WT is wild-type.

FIG. 14B shows Southern Blot results of Mutant allele 1 positive clones digested with ScaI and hybridized with 3′ probe. M is a marker. WT is wild-type.

FIG. 14C shows Southern Blot results of Mutant allele 1 positive clones digested with XmnI and hybridized with A probe. M is a marker. WT is wild-type.

FIG. 14D shows Southern Blot results of Mutant allele 1 positive clones digested with BglII and hybridized with 5′ probe. M is a marker. WT is wild-type.

FIG. 15 shows PCR assay results using primers DE-F1 and DE-RI to verify the knock-out of the sequence from Cμ to Co in Mutant allele 2.

FIG. 16 shows PCR assay results using primers GT-Mut-F, GT-Mut-R and GT-WT-R to verify the knock-out of the sequence from Cμ to Sγ1 in Mutant allele 2′.

FIG. 17 shows PCR assay results using primers GT-3F and GT-3R to verify the knock-out of the CH1 coding sequence of Cμ in Mutant Allele 3.

FIG. 18A shows PCR assay results using primers Mut-F and Mut-R to verify the sequence of CμΔCH1 in Mutant Allele 4. WT is a wild-type control. H₂O is a blank control.

FIG. 18B shows PCR assay results using primers F4 and R4 to verify the absence of Co in Mutant Allele 4.

FIGS. 19A-19B show PCR assay results using primer pairs Mut-F/Mut-R and F3/R3 to verify the sequence of CμΔCH1 and CδΔCH1, respectively, in Mutant allele 5. WT is a wild-type control. H₂O is a blank control.

FIG. 20 is an exemplary flow chart of a method of introducing human immunoglobulin genes into the mouse genome.

FIG. 21 is an overview of replacing mouse immunoglobulin heavy chain variable region locus sequences with human immunoglobulin heavy chain variable region locus sequences.

FIG. 22 shows the length distribution of CDR3 in the heavy chain variable region of antibodies produced by immunizing heterozygous Mut3 mice with antigen A.

FIG. 23 shows the germline gene usage of the variable region genes in mice heterozygous for heavy chain Mutant allele 3 genotype.

FIG. 24 shows the KD value distribution of antibodies produced in heterozygous Mut3 mice (H/−) against antigen A.

FIG. 25 shows the KD value distribution of antibodies produced in homozygous Mut2 mice against antigen A.

FIG. 26 shows the germline gene usage of the variable region genes in homozygous Mut2 mice.

FIG. 27 is a schematic diagram showing human immunoglobulin heavy chain (IGH) locus on chromosome 14 (14q32.33).

FIG. 28 is a schematic diagram showing mouse (Mus musculus) IGH locus on chromosome 12 (12F2) (strain C57BL/6).

FIG. 29 lists IMGT repertoire for human heavy chain immunoglobulin locus (IGH).

FIG. 30 lists IMGT repertoire for mouse IGH.

FIG. 31 lists sequences discussed in the disclosure.

FIG. 32 shows Western blot results of serum IgG levels in mice with Mutant allele 2′, Mutant allele 3, and Mutant allele 4 genotypes, respectively. Biot. Ladder is a protein marker. WT represents a wild-type mouse.

FIG. 33 shows the length distribution of CDR3 in the heavy chain variable region of antibodies produced by immunizing heterozygous Mut3 mice with human 4-1BB.

FIG. 34 shows the germline gene usage of the variable region genes in mice homozygous for heavy chain Mutant allele 3 genotype.

FIG. 35 shows the length distribution of CDR3 in the heavy chain variable region of antibodies produced by immunizing heterozygous Mut3 mice with human CD3ED and cynomolgus CD3ED.

FIG. 36 shows the germline gene usage of the variable region genes in mice homozygous for heavy chain Mutant allele 3 genotype.

FIG. 37 lists CDR sequences of heavy chain variable region of anti-TFR1 antibodies according to Kabat numbering.

FIG. 38 lists CDR sequences of heavy chain variable region of anti-TFR1 antibodies according to IMGT numbering.

FIG. 39 list amino acid sequences of the heavy chain variable region of the anti-TFR1 antibodies.

FIG. 40A shows the antibody concentration in total brain protein of hTFR1 mice within 72 hours of intravenous (i.v.) administration of hIgG1 (G1), JR141-N (G2), 23B8-N (G3), 24A1-N (G4), 24G5-N (G5), or 24C9-N (G6) by.

FIG. 40B shows the ratio of antibody concentration in brain total protein to serum antibody concentration of hTFR1 mice within 72 hours of intravenous (i.v.) administration of hIgG1 (G1), JR141-N (G2), 23B8-N (G3), 24A1-N (G4), 24G5-N (G5), or 24C9-N (G6).

FIG. 40C shows the antibody concentration in brain parenchyma of hTFR1 mice within 72 hours of intravenous (i.v.) administration of hIgG1 (G1), JR141-N (G2), 23B8-N (G3), 24A1-N (G4), 24G5-N (G5), or 24C9-N (G6).

FIG. 40D shows the ratio of antibody concentration in brain parenchyma to serum antibody concentration of hTFR1 mice within 72 hours of intravenous (i.v.) administration of hIgG1 (G1), JR141-N (G2), 23B8-N (G3), 24A1-N (G4), 24G5-N (G5), or 24C9-N (G6).

FIG. 41A shows the antibody concentration test results in brain parenchyma after 24 hours of intravenous (i.v.) administration of hIgG1 (G1), JR141-N (G2), 23B8-N (G3), 24A1-N (G4), or 24G5-N (G5).

FIG. 41B shows the antibody concentration test results in brain total protein (Whole Brain) after 24 hours of intravenous (i.v.) administration of hIgG1 (G1), JR141-N (G2), 23B8-N (G3), 24A1-N (G4), or 24G5-N (G5).

FIG. 42 shows the antibody concentration results after 6 hours or 24 hours of intravenous (i.v.) administration of JR141-N (G2-G4) or 24G5-N (G5-G7)). hIgG1 was used as a negative control;

FIG. 43 shows the germline gene usage of the variable region genes in mice homozygous for heavy chain Mutant allele 3 genotype;

FIG. 44 shows the germline gene usage of the variable region genes in heterozygous Mut2′ mice (H/−);

FIG. 45 shows the KD value distribution of antibodies produced in heterozygous Mut2′ mice (H/−) against Human Serum Albumin;

FIG. 46 shows the germline gene usage of the variable region genes in mice homozygous for heavy chain Mutant allele 4 genotype;

FIG. 47 shows the germline gene usage of the variable region genes in mice homozygous for heavy chain Mutant allele 5 genotype.

### DETAILED DESCRIPTION

The present disclosure relates to genetically modified animals and methods for producing heavy-chain antibodies.

A heavy-chain antibody (or heavy chain-only antibody) is an antibody which has only heavy chains (generally two heavy chains) and lacks the two light chains usually found in antibodies. Naturally occurring heavy-chain antibodies have been discovered in cartilaginous fishes (e.g., shark) and camelids (e.g., llama). For example, in cartilaginous fishes, the immunoglobulin new antigen receptor (IgNAR) is a heavy-chain antibody. IgNAR shows significant structural differences to other antibodies. It has five constant domains (CH) per chain instead of the usual three, several disulfide bonds in unusual positions, and the complementarity-determining region 3 (CDR3) forms an extended loop covering the site which binds to a light chain in other antibodies. These differences, in combination with the phylogenetic age of the cartilaginous fishes, have led to the hypothesis that IgNAR could be more closely related to a primordial antigen-binding protein than the mammalian immunoglobulins.

The only mammals with heavy-chain (IgG-like) antibodies are camelids such as dromedaries, camels, llamas and alpacas. Like all mammals, camelids (e.g., llamas) can produce conventional antibodies made of two heavy chains and two light chains bound together with disulfide bonds in a Y shape (e.g., IgG1). However, they also produce two unique subclasses of IgG: IgG2 and IgG3, also known as heavy chain IgG. These antibodies are made of only two heavy chains, which lack the CH1 region but still bear an antigen-binding domain (e.g., VHH) at their N-terminus. Conventional Ig require the association of variable regions from both heavy and light chains to allow a high diversity of antigen-antibody interactions. Although isolated heavy and light chains still show this capacity, they exhibit very low affinity when compared to paired heavy and light chains. The unique feature of heavy chain IgG is the capacity of their monomeric antigen binding regions to bind antigens with specificity, affinity and especially diversity that are comparable to conventional antibodies without the need of pairing with another region. This feature is mainly due to a couple of major variations within the amino acid sequence of the variable region of the two heavy chains, which induce deep conformational changes when compared to conventional Ig. Major substitutions in the variable regions prevent the light chains from binding to the heavy chains, but also prevent unbound heavy chains from being recycled by the Immunoglobulin Binding Protein.

The single variable domain of these heavy-chain antibodies (designated VHH, sdAb, or nanobody) is the smallest antigen-binding domain generated by adaptive immune systems. The Complementarity Determining Region 3 (CDR3) of the variable region of these antibodies has often been found to be twice as long as the conventional ones. This results in an increased interaction surface with the antigen as well as an increased diversity of antigen-antibody interactions, which compensates the absence of the light chains. With a long complementarity-determining region 3 (CDR3), VHHs can extend into crevices on proteins that are not accessible to conventional antibodies, including functionally interesting sites such as the active site of an enzyme or the receptor-binding canyon on a virus surface. Moreover, an additional cysteine residue allows the structure to be more stable, thus increasing the strength of the interaction.

VHHs offer numerous other advantages compared to conventional antibodies carrying variable domains (VH and VL) of conventional antibodies, including higher stability, solubility, expression yields, and refolding capacity, as well as better in vivo tissue penetration. Moreover, in contrast to the VH domains of conventional antibodies, VHH do not display an intrinsic tendency to bind to light chains. This facilitates the induction of heavy chain antibodies in the presence of a functional light chain loci. Further, since VHH do not bind to VL domains, it is much easier to reformat VHHs into bispecific antibody constructs than constructs containing conventional VH-VL pairs or single domains based on VH domains.

A notable difference between the camelid VHH and the human VH domain is the length and orientation of the CDR3 loop. The CDR3 corresponds to the unique region of the antibody molecule that is encoded by a DNA element newly generated during B-cell development. Genetic recombination results in the fusion of a D-element with flanking V- and J-elements. During recombination further genetic diversity is generated by addition and/or deletion of nucleotides at the junctions. Thereby, the CDR3 loop provides the major contribution to antibody diversity and specificity. A limited number of variable region genes (IGHV, IGHD, and IGHJ) in some early transgenic heavy chain antibody animals, results in some antigens not being recognized by these animals, despite potent antigen response by wildtype animals (Janssens, Rick, et al. “Generation of heavy-chain-only antibodies in mice.” Proceedings of the National Academy of Sciences 103.41 (2006): 15130-15135). The present disclosure provides genetically modified animals that have complete human heavy chain antibody repertoires. Thus, the variable domains generated by these animals can have the maximal possible diversity for human heavy chain variable domains, thus maximizing the chance to obtain a fully humanized heavy chain antibody.

Furthermore, because the entire sequence at the human immunoglobulin locus is introduced into the animal genome (with no modifications or limited modifications), these genes can undergo the V (D) J recombination in a way that is very similar to what happens in human, reducing the risk of the generating new immunogenic epitopes that can be recognized in a human immune system, thereby decreasing immunogenicity. The immunogenicity can lead to production of anti-drug-antibodies and may comprise efficacy. Here, the endogenous IGHV, IGHD, and IGHJ genes have been effectively deleted. It is less likely that the antibodies generated by the antibody repertoires are immunogenic in humans. In addition, the antibody production can be very efficient and has a production rate that is similar to the normal production rates due to the efficient V (D) J recombination. Thus, the antibodies are more suitable as therapeutics in humans. Therefore, the genetically modified animals provide an advantageous platform to produce humanized heavy chain antibodies.

In addition, IgG1 is the most abundant antibody subtype in serum, with long serum half-life, strong FcγR affinity, antibody-dependent cellular cytotoxicity (ADCC), complement dependent cytotoxicity (CDC) activity, etc. IgG1 has unique advantages in the field of antibody drug development. Thus, in one aspect, the present disclosure is particularly related to preparation of humanized mice that can produce heavy-chain antibodies of IgG1 subtype. In the meantime, coding sequences for all other IgG subtypes can be deleted. This creates an efficient and reliable platform to create heavy chain antibodies in the animals.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, heavy chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)2, and Fv fragments.

As used herein, the term “human antibody” refers to an antibody that is encoded by a nucleic acid (e.g., rearranged human immunoglobulin heavy or light chain locus) present in a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a mouse) containing an unrearranged or rearranged human immunoglobulin locus (e.g., heavy or light chain human immunoglobulin locus).

As used herein, the term “chimeric antibody” refers to an antibody that contains a sequence present in at least two different antibodies (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a human antibody and the constant domains of a non-human antibody. Additional examples of chimeric antibodies are described herein and are known in the art.

As used herein, the term “humanized antibody” refers to a non-human antibody which contains sequence derived from a non-human (e.g., mouse) immunoglobulin and contains sequences derived from a human immunoglobulin.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen.

As used herein, the term “heavy-chain antibody” refers to an antibody molecule which is composed only of heavy chains (generally two) and does not have any light chains.

As used herein, the term “VHH” refers to the variable domain derived from a heavy-chain antibody. The VHH can specifically recognize an antigen without the need to be paired with a VL. In some embodiments, the VHH (also know as sdAb or nanobody) described herein is derived from any of the humanized heavy-chain antibody described herein. In some embodiments, the VHH, sdAb, or nanobody described herein is derived from the heavy chain antibody produced by any of the genetically modified non-human animal described herein.

As used herein, the terms “subject” and “patient” are used interchangeably throughout the specification and describe an animal, human or non-human. Veterinary and non-veterinary applications are contemplated by the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody.

As used herein, the terms “polypeptide,” “peptide.” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

As used herein, the term “an unmodified human sequence” refers to a sequence that is derived from a human subject, a human cell, a cultured human cell or a human cell line, wherein the sequence is identical to the genetic sequence of a human subject, a human cell, a cultured human cell or a human cell line.

### Immunoglobulin Heavy Chain Constant Region Locus

Heavy chain immunoglobulin locus (also known as IGH or immunoglobulin heavy locus) is a region on the chromosome (e.g., mouse chromosome 12) that contains genes for the heavy chains of antibodies (or immunoglobulins). It includes sequences encoding the heavy chain variable region and heavy chain constant region.

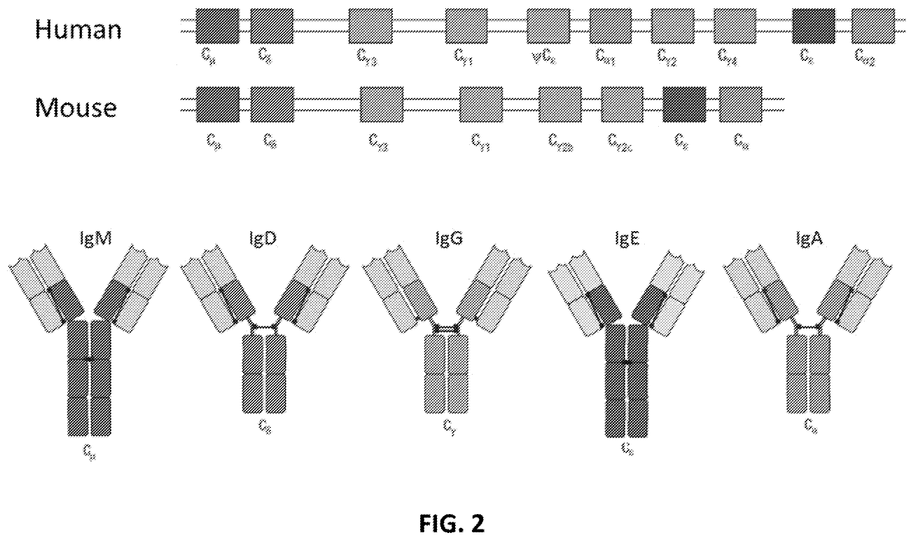

As shown in FIG. 2, the mouse immunoglobulin heavy chain constant region genes include (as shown in the following order): immunoglobulin heavy constant mu (IGHM, or Cμ), immunoglobulin heavy constant delta (IGHδ, or Cδ), immunoglobulin heavy constant gamma 3 (IGHG3, or Cγ3), immunoglobulin heavy constant gamma 1 (IGHG1, or Cγ1), immunoglobulin heavy constant gamma 2b (IGHG2b, or Cγ2b), immunoglobulin heavy constant gamma 2a (IGHG2c, or Cγ2c), immunoglobulin heavy constant epsilon (IGHE, or Cε), and immunoglobulin heavy constant alpha (IGHA, or Cα) genes. In some embodiments, immunoglobulin heavy constant gamma 2a (IGHG2a) is at the position of IGHG2c. In contrast, human immunoglobulin constant region genes include (as shown in the following order): immunoglobulin heavy constant mu (IGHM, or Cμ), immunoglobulin heavy constant delta (IGHδ, Cδ), immunoglobulin heavy constant gamma 3 (IGHG3, or Cγ3), immunoglobulin heavy constant gamma 1 (IGHG1, or Cγ1), immunoglobulin heavy constant epsilon P1 (pseudogene) (IGHEP1, or ψCε), immunoglobulin heavy constant alpha 1 (IGHA1, or Cα1), immunoglobulin heavy constant gamma P (non-functional) (IGHGP, or CγP; not shown), immunoglobulin heavy constant gamma 2 (IGHG2, or Cγ2), immunoglobulin heavy constant gamma 4 (IGHG4, Cγ4), immunoglobulin heavy constant epsilon (IGHE, or Cε), and immunoglobulin heavy constant alpha 2 (IGHA2, or Cα2) genes.

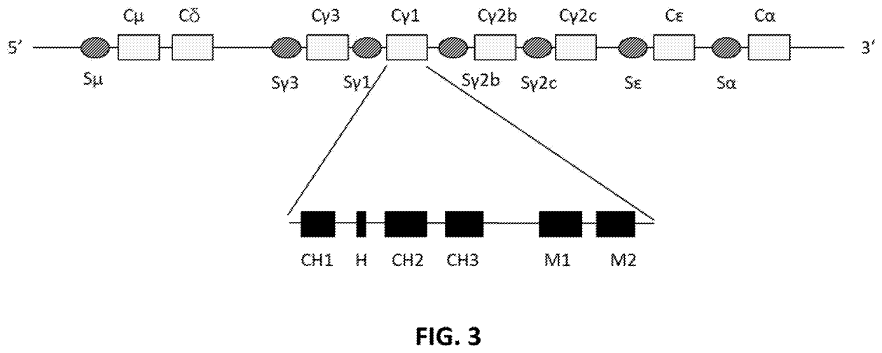

Immunoglobulin class switching (or isotype switching, or isotypic commutation, or class switch recombination (CSR)) is a biological mechanism that changes a B cell's production of antibody from one class to another; for example, from isotype IgM to isotype IgG. During this process, the constant region portion of the antibody-heavy chain is changed, but the variable region of the heavy chain stays the same. Since the variable region does not change, class switching does not affect antigen specificity. Instead, the antibody retains affinity for the same antigens, but can interact with different effector molecules. This allows different daughter cells from the same activated B cell to produce antibodies of different isotypes or subtypes (e.g. IgG1, IgG2 etc.). Class switching occurs by a mechanism called class switch recombination (CSR) binding. Class switch recombination is a biological mechanism that allows the class of antibody produced by an activated B cell to change during a process known as isotype or class switching. During CSR, portions of the antibody-heavy chain locus are removed from the chromosome, and the gene segments surrounding the deleted portion are rejoined to retain a functional antibody gene that produces antibody of a different isotype. Double-stranded breaks are generated in DNA at conserved nucleotide motifs, called switch(S) regions, which are upstream from gene segments that encode the constant regions of antibody heavy chains; these occur adjacent to all heavy chain constant region genes with the exception of Co. DNA is nicked and broken at two selected switch regions by the activity of a series of enzymes, including Activation-Induced (Cytidine) Deaminase (AID), uracil DNA glycosylase and apyrimidinic/apurinic (AP)-endonucleases. The intervening DNA between the switch regions is subsequently deleted from the chromosome, e.g., removing unwanted Cμ or C8 heavy chain constant region sequences and allowing substitution of Cγ, Cα, or Cε constant region gene segment. The free ends of the DNA are rejoined by a process called non-homologous end joining (NHEJ) to link the variable domain exon to the desired downstream constant domain exon of the antibody heavy chain. In the absence of non-homologous end joining, free ends of DNA can be rejoined by an alternative pathway biased toward microhomology joins. With the exception of the Cμ and Cδ genes, only one antibody class is expressed by a B cell at any point in time. FIG. 3 shows the location of each switch region (e.g., Sμ, Sγ3, Sγ1, Sγ2b, Sγ2c, Sε, and Sα) in the mouse immunoglobulin heavy chain constant region locus. Specifically, the Cγ1 gene comprises exons encoding the CH1 domain, the hinge region, the CH2 domain, the CH3 domain, and two transmembrane domains. The exons are labelled with CH1, H, CH2, CH3, M1, and M2, respectively.

The five main classes of immunoglobulins are IgM, IgD, IgG, IgE, and IgA, each of which can occur as transmembrane antigen receptors or secreted antibodies. In humans, IgG is found as four subclasses (IgG1, IgG2, IgG3, and IgG4), named by decreasing order of their abundance in serum, and IgA antibodies are found as two subclasses (IgAQ1 and IgA2). The different heavy chains that define these classes are known as isotypes and are designated by the lowercase Greek letters μ (IgM), δ (IgD), γ (IgG), ε (IgE), and α (IgA), respectively.

Although the position and number of disulfide bonds are different between IgG1-IgG4, structures of the four IgG subtypes are very similar. In fact, as the most abundant IgG subtype in plasma, IgG1 is widely used for making recombinant therapeutic antibodies. In contrast, IgG3 is rarely used for antibody drug development, because it has a weak binding affinity to FcRn and a short half-life (about 9 days). Therefore, IgG3-based antibody drugs have to be administered more frequently for pharmacokinetics reasons. In addition, the antibody levels of different subtypes can change during body development, which is described e.g., in Elena Blanco et al., “Age-associated distribution of normal B-cell and plasma cell subsets in peripheral blood,” Journal of Allergy and Clinical Immunology, Volume 141, Issue 6 (2018), which is incorporated herein by reference in its entirety. IgG1 is the most abundant antibody subtype in serum, with long serum half-life, strong FcγR affinity, antibody-dependent cellular cytotoxicity (ADCC), complement dependent cytotoxicity (CDC) activity, etc. IgG1 has unique advantages in the field of antibody drug development. Thus, in one aspect, the disclosure is related to preparation of humanized mice that can produce heavy-chain antibodies of IgG1 subtype. In one aspect, the present disclosure is particularly related to preparation of humanized mice that can produce heavy-chain antibodies of IgG1 subtype. In some embodiments, the heavy-chain antibodies can be further processed to generate nanobodies.

In one aspect, the disclosure relates to a genetically modified non-human animal comprising a modified immunoglobulin heavy chain locus, wherein the modified immunoglobulin heavy chain locus comprises an IgG constant region gene, wherein the IgG constant region gene encodes an IgG heavy chain constant region lacking a CH1 domain, wherein the genetically modified non-human animal expresses a heavy-chain antibody.

In some embodiments, the IgG constant region gene is one of the following: IGHG3, IGHG1, IGHG2a, IGHG2b, and IGHG2c. In some embodiments, the modified immunoglobulin heavy chain locus has one, two, three, four, or five IgG constant region genes, wherein none of them encodes a CH1 domain. In some embodiments, only one, two, or three IGHG genes do not encode a CH1 domain, while at least one, two, or three remaining IGHG genes can encode a CH1 domain. In some embodiments, the modified immunoglobulin heavy chain locus has only one (e.g., exactly one) IgG constant region gene, e.g., IGHG1. In some embodiments, the modified immunoglobulin heavy chain locus does not include IGHG3, IGHG2a, IGHG2b, and/or IGHG2c genes.

In some embodiments, the modified immunoglobulin heavy chain locus has only one IGHG gene (e.g., IGHG3, IGHG1, IGHG2a, IGHG2b, or IGHG2c), and the sequence encoding the CH1 domain in the IGHG is deleted. In some embodiments, the IGHG gene is operably linked to Sγ3, Sγ1, Sγ2a, Sγ2b, or Sγ2c (e.g., Sγ3 or Sγ1).

In some embodiments, the IGHM is an intact functional endogenous IGHM gene. In some embodiments, the sequence encoding the CH1 domain in the IGHM is deleted. In some embodiments, IGHM is deleted. In some embodiments, the IGHδ is an intact functional endogenous IGHδ gene. In some embodiments, the sequence encoding the CH1 domain in the IGHδ is deleted. In some embodiments, IGHδ is deleted. In some embodiments, both IGHM and IGHδ are deleted.

In some embodiments, the IGHE is an intact functional endogenous IGHE gene. In some embodiments, the sequence encoding the CH1 domain in the IGHE is deleted. In some embodiments, IGHE is deleted.

In some embodiments, the IGHA is an intact functional endogenous IGHA gene. In some embodiments, the sequence encoding the CH1 domain in the IGHA is deleted. In some embodiments, IGHA is deleted.

In various embodiments, the humanized heavy chain antibody comprises a unique immunoglobulin constant region (Fc), that lacks at least the CH1 domain. In some embodiment, it also lacks the hinge region of a human Fc. In some embodiment, the heavy-chain antibody comprises the CH2 and CH3 regions of an immunoglobulin G (IgG) heavy chain constant region. In some embodiments, the constant region of the heavy chain antibody includes the hinge, CH2 and CH3 regions of the IgG heavy chain Fc.

In some embodiments, a suitable number of rearranged heavy chain variable regions that can effectively survive selection when presented during B cell development is needed. In one aspect, the disclosure provides a transgenic animal comprising a germline genetic modification that comprises a deletion of a nucleotide sequence encoding a CH1 domain of an IgG, wherein the animal expresses a functional IgM and the animal expresses in its serum an IgG heavy chain antibody (e.g., with IgG1 heavy chain CH2 and CH3 domains). In some embodiments, the IgM comprises two heavy chains, and they associate with two lambda or kappa lights chains to recognize an antigen. In some embodiments, the functional IgM includes a CH1 domain. In some embodiments, the functional IgM does not include a CH1 domain. While not intending to be bound by any theory, it is believed that the deleting a sequence encoding a CH1 domain from endogenous IGHM gene does not substantially change the IgM function.

In some embodiments, the modified immunoglobulin heavy chain constant region locus includes a modified IGHδ gene lacking a sequence encoding a CH1 domain. In some embodiments, the modified IGHδ expresses a functional IgD. While not intending to be bound by any theory, it is believed that the deleting a sequence encoding a CH1 domain from endogenous IGHδ gene does not substantially change the IgD function.

### Immunoglobulin Heavy Chain Variable Region Locus

To produce a humanized heavy chain antibody (HCAb), the heavy chain immunoglobulin variable region locus in the animal can be humanized. This heavy chain immunoglobulin variable region represents the germline organization of the heavy chain locus. The locus includes V (variable), D (diversity), J (joining), and C (constant) segments. The genes in the V region form a V gene cluster (also known as IGHV gene cluster). The genes in the D region form a D gene cluster (also known as IGHD gene cluster). The genes in the J region form a J gene cluster (also known as IGHJ gene cluster).

During B cell development, a recombination event at the DNA level joins a single D segment (also known as an IGHD gene) with a J segment (also known as an IGHJ gene); the fused D-J exon of this partially rearranged D-J region is then joined to a V segment (also known as an IGHV gene). The rearranged V-D-J region containing a fused V-D-J exon is then transcribed and fused at the RNA level to the IGHM constant region; this transcript encodes a mu heavy chain. Later in development, B cells generate V-D-J-Cμ-Cδ pre-messenger RNA, which is alternatively spliced to encode either a mu or a delta heavy chain. Mature B cells in the lymph nodes undergo switch recombination, so that the fused V-D-J gene segment is brought in proximity to one of the IGHG, IGHA, or IGHE gene segments and each cell expresses either the gamma, alpha, or epsilon heavy chain. Potential recombination of many different IGHV genes with several IGHJ genes provides a wide range of antigen recognition. Additional diversity is attained by junctional diversity, resulting from the random addition of nucleotides by terminal deoxynucleotidyl transferase, and by somatic hypermutation, which occurs during B cell maturation in the spleen and lymph nodes. Several V, D, J, and C segments are known to be incapable of encoding a protein and are considered pseudogenous gene segments (often simply referred to as pseudogenes).

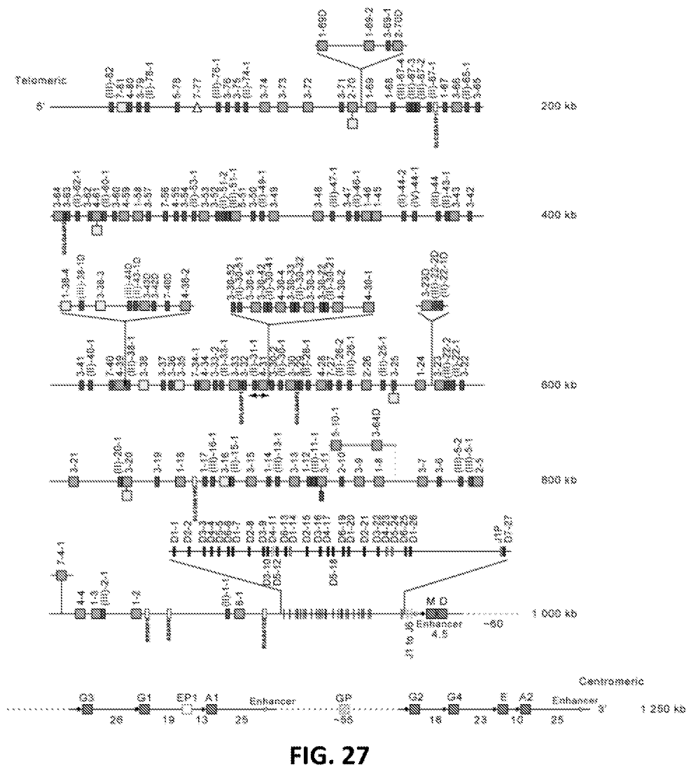

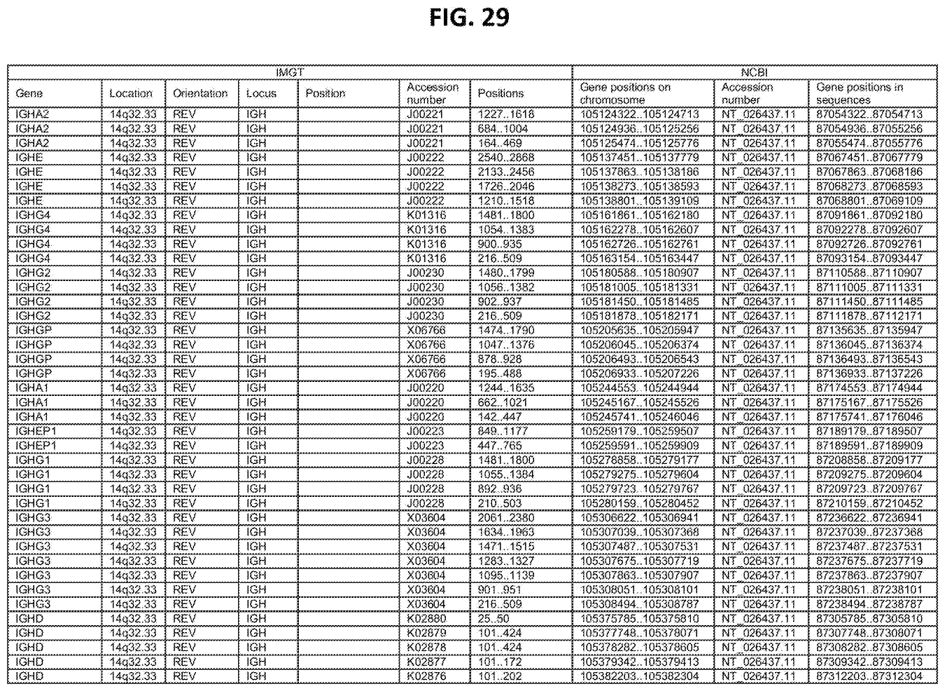

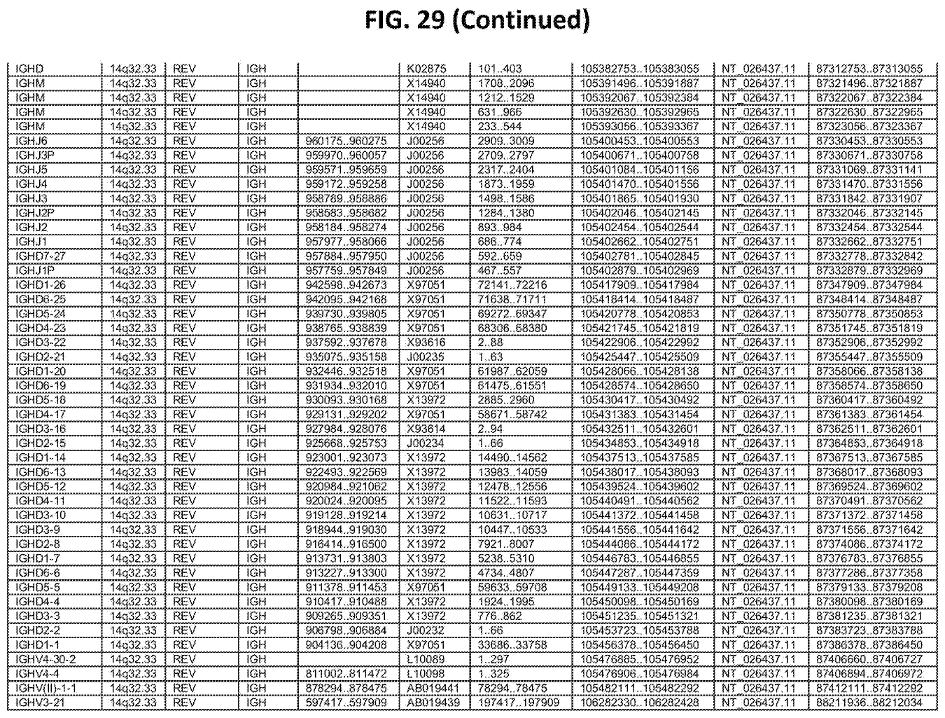

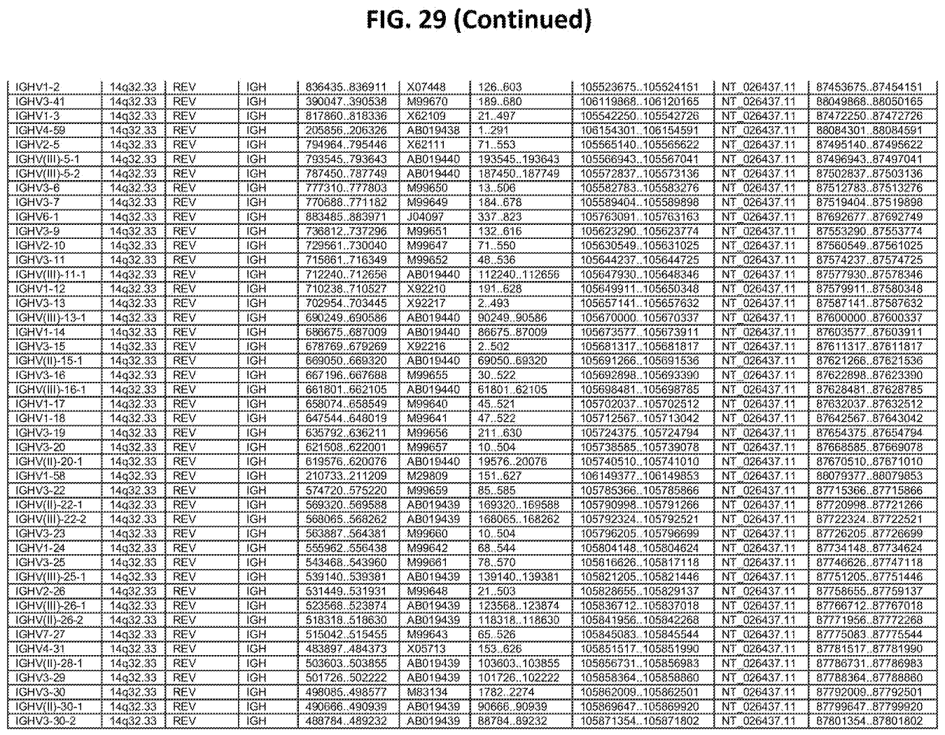

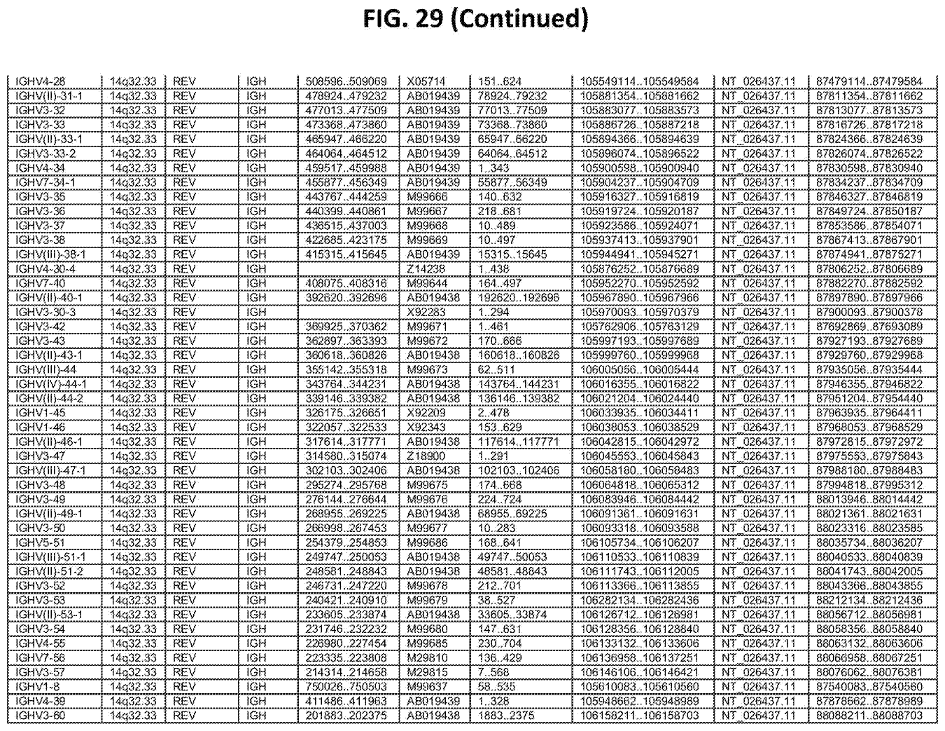

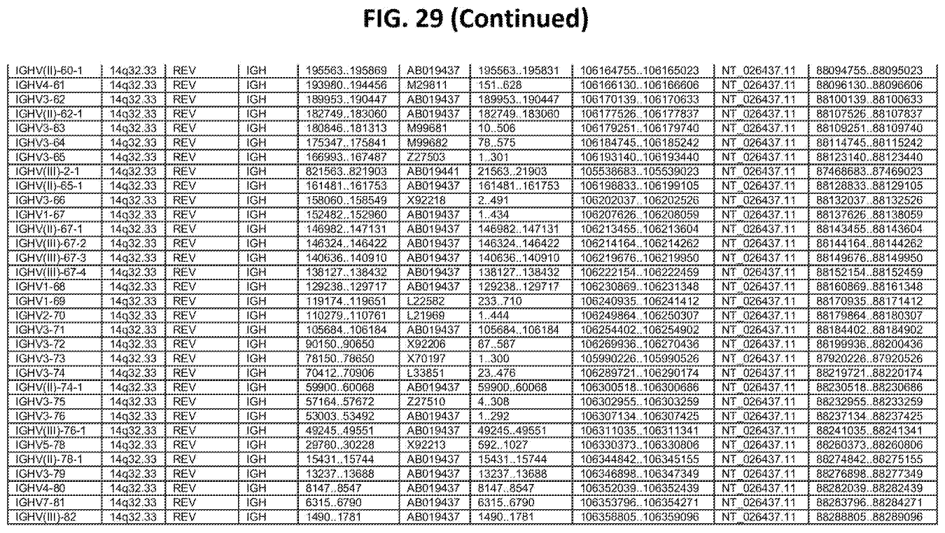

The human heavy chain immunoglobulin locus is located on human chromosome 14 (FIG. 27 and FIG. 29). Table 1 lists IGHV genes and its relative orders in this locus.

TABLE 1
List of IGHV genes on human chromosome 14

| Gene names | Order |
|---|---|
| IGHV(III)-82 | 1 |
| IGHV7-81 | 2 |
| IGHV4-80 | 3 |
| IGHV3-79 | 4 |
| IGHV(II)-78-1 | 5 |
| IGHV5-78 | 6 |
| IGHV7-77 | 7 |
| IGHV(III)-76-1 | 8 |
| IGHV3-76 | 9 |
| IGHV3-75 | 10 |
| IGHV(II)-74-1 | 11 |
| IGHV3-74 | 12 |
| IGHV3-73 | 13 |
| IGHV3-72 | 14 |
| IGHV3-71 | 15 |
| IGHV2-70 | 16 |
| IGHV1-69D | 17 |
| IGHV1-69-2 | 18 |
| IGHV3-69-1 | 19 |
| IGHV2-70D | 20 |
| IGHV1-69 | 21 |
| IGHV1-68 | 22 |
| IGHV(III)-67-4 | 23 |
| IGHV(III)-67-3 | 24 |
| IGHV(III)-67-2 | 25 |
| IGHV(II)-67-1 | 26 |
| SLC20A1P1 | 27 |
| (GLVR1) | |
| IGHV1-67 | 28 |
| IGHV3-66 | 29 |
| IGHV(II)-65-1 | 30 |
| IGHV3-65 | 31 |
| IGHV3-64 | 32 |
| GOLGA4P3 | 33 |
| (Golgin) | |
| IGHV3-63 | 34 |
| IGHV(II)-62-1 | 35 |
| IGHV3-62 | 36 |
| IGHV4-61 | 37 |
| IGHV(II)-60-1 | 38 |
| IGHV3-60 | 39 |
| IGHV4-59 | 40 |
| IGHV1-58 | 41 |
| IGHV3-57 | 42 |
| IGHV7-56 | 43 |
| IGHV4-55 | 44 |
| IGHV3-54 | 45 |
| IGHV(II)-53-1 | 46 |
| IGHV3-53 | 47 |
| IGHV3-52 | 48 |
| IGHV(II)-51-2 | 49 |
| IGHV(III)-51-1 | 50 |
| IGHV5-51 | 51 |
| IGHV3-50 | 52 |
| IGHV(II)-49-1 | 53 |
| IGHV3-49 | 54 |
| IGHV3-48 | 55 |
| IGHV(III)-47-1 | 56 |
| IGHV3-47 | 57 |
| IGHV(II)-46-1 | 58 |
| IGHV1-46 | 59 |
| IGHV1-45 | 60 |
| IGHV(II)-44-2 | 61 |
| IGHV(IV)-44-1 | 62 |
| IGHV(III)-44 | 63 |
| IGHV(II)-43-1 | 64 |
| IGHV3-43 | 65 |
| IGHV3-42 | 66 |
| IGHV3-41 | 67 |
| IGHV(II)-40-1 | 68 |
| IGHV7-40 | 69 |
| IGHV4-39 | 70 |
| IGHV1-38-4 | 71 |
| IGHV(III)-38- | 72 |
| 1D | |
| IGHV3-38-3 | 73 |
| IGHV(III)-44D | 74 |
| IGHV(II)-43- | 75 |
| 1D | |
| IGHV3-43D | 76 |
| IGHV3-42D | 77 |
| IGHV7-40D | 78 |
| IGHV4-38-2 | 79 |
| IGHV(III)-38-1 | 80 |
| IGHV3-38 | 81 |
| IGHV3-37 | 82 |
| IGHV3-36 | 83 |
| IGHV3-35 | 84 |
| IGHV7-34-1 | 85 |
| IGHV4-34 | 86 |
| IGHV3-33-2 | 87 |
| IGHV(II)-33-1 | 88 |
| IGHV3-33 | 89 |
| GOLGA4P1 | 90 |
| (Golgin) | |
| IGHV3-32 | 91 |
| IGHV(II)-31-1 | 92 |
| IGHV4-31 | 93 |
| IGHV3-30-52 | 94 |
| IGHV(II)-30-51 | 95 |
| IGHV3-30-5 | 96 |
| IGHV3-30-42 | 97 |
| IGHV(II)-30-41 | 98 |
| IGHV4-30-4 | 99 |
| IGHV3-30-33 | 100 |
| IGHV(II)-30-32 | 101 |
| IGHV3-30-3 | 102 |
| IGHV3-30-22 | 103 |
| IGHV(II)-30-21 | 104 |
| IGHV4-30-2 | 105 |
| IGHV4-30-1 | 106 |
| IGHV3-30-2 | 107 |
| IGHV(II)-30-1 | 108 |
| IGHV3-30 | 109 |
| GOLGA4P2 | 110 |
| (Golgin) | |
| IGHV3-29 | 111 |
| IGHV(II)-28-1 | 112 |
| IGHV4-28 | 113 |
| IGHV7-27 | 114 |
| IGHV(II)-26-2 | 115 |
| IGHV(III)-26-1 | 116 |
| IGHV2-26 | 117 |
| IGHV(III)-25-1 | 118 |
| IGHV3-25 | 119 |
| IGHV1-24 | 120 |
| IGHV3-23D | 121 |
| IGHV(III)-22- | 122 |
| 2D | |
| IGHV(II)-22-1D | 123 |
| IGHV3-23 | 124 |
| IGHV(III)-22-2 | 125 |
| IGHV(II)-22-1 | 126 |
| IGHV3-22 | 127 |
| IGHV3-21 | 128 |
| IGHV(II)-20-1 | 129 |
| IGHV3-20 | 130 |
| IGHV3-19 | 131 |
| IGHV1-18 | 132 |
| SLC20A1P2 | 133 |
| IGHV1-17 | 134 |
| IGHV(III)-16-1 | 135 |
| IGHV3-16 | 136 |
| IGHV(II)-15-1 | 137 |
| IGHV3-15 | 138 |
| IGHV1-14 | 139 |
| IGHV(III)-13-1 | 140 |
| IGHV3-13 | 141 |
| IGHV1-12 | 142 |
| IGHV(III)-11-1 | 143 |
| IGHV3-11 | 144 |
| IGHV2-10 | 145 |
| IGHV3-9 | 146 |
| IGHV1-8 | 147 |
| IGHV5-10-1 | 148 |
| IGHV3-64D | 149 |
| IGHV3-7 | 150 |
| IGHV3-6 | 151 |
| IGHV(III)-5-2 | 152 |
| IGHV(III)-5-1 | 153 |
| IGHV2-5 | 154 |
| IGHV7-4-1 | 155 |
| IGHV4-4 | 156 |
| IGHV1-3 | 157 |
| IGHV(III)-2-1 | 158 |
| IGHV1-2 | 159 |
| | * |
| | * |
| IGHV(II)-1-1 | 162 |
| IGHV6-1 | 163 |
| | * |

RPS8P1, ADAM6, and KIAA0125 are also located in this locus. The relative order of RPS8P1 is 160, the relative order of ADAM6 is 161, and the relative order of KIAA0125 is 164. Table 2 lists all IGHD genes and its relative orders on human chromosome 14. Table 3 lists all IGHJ genes and its relative orders on human chromosome 14. The genes for immunoglobulin constant domains are located after the IGHV, IGHD, and IGHJ genes. These genes include (as shown in the following order): immunoglobulin heavy constant mu (IGHM), immunoglobulin heavy constant delta (IGH 8), immunoglobulin heavy constant gamma 3 (IGHG3), immunoglobulin heavy constant gamma 1 (IGHG1), immunoglobulin heavy constant epsilon P1 (pseudogene) (IGHEP1), immunoglobulin heavy constant alpha 1 (IGHA1), immunoglobulin heavy constant gamma P (non-functional) (IGHGP), immunoglobulin heavy constant gamma 2 (IGHG2), immunoglobulin heavy constant gamma 4 (IGHG4), immunoglobulin heavy constant epsilon (IGHE), and immunoglobulin heavy constant alpha 2 (IGHA2). These genes and the order of these genes are also shown in FIG. 27 and FIG. 29.

TABLE 2
List of IGHD genes on human chromosome 14

| Gene names | Order |
|---|---|
| IGHD1-1 | 165 |
| IGHD2-2 | 166 |
| IGHD3-3 | 167 |
| IGHD4-4 | 168 |
| IGHD5-5 | 169 |
| IGHD6-6 | 170 |
| IGHD1-7 | 171 |
| IGHD2-8 | 172 |
| IGHD3-9 | 173 |
| IGHD3-10 | 174 |
| IGHD4-11 | 175 |
| IGHD5-12 | 176 |
| IGHD6-13 | 177 |
| IGHD1-14 | 178 |
| IGHD2-15 | 179 |
| IGHD3-16 | 180 |
| IGHD4-17 | 181 |
| IGHD5-18 | 182 |
| IGHD6-19 | 183 |
| IGHD1-20 | 184 |
| IGHD2-21 | 185 |
| IGHD3-22 | 186 |
| IGHD4-23 | 187 |
| IGHD5-24 | 188 |
| IGHD6-25 | 189 |
| IGHD1-26 | 190 |
| | * |
| IGHD7-27 | 192 |

TABLE 3
List of IGHJ genes on human chromosome 14

| Gene names | Order |
|---|---|
| IGHJ1P | 191 |
| IGHJ1 | 193 |
| IGHJ2 | 194 |
| IGHJ2P | 195 |
| IGHJ3 | 196 |
| IGHJ4 | 197 |
| IGHJ5 | 198 |
| IGHJ3P | 199 |
| IGHJ6 | 200 |

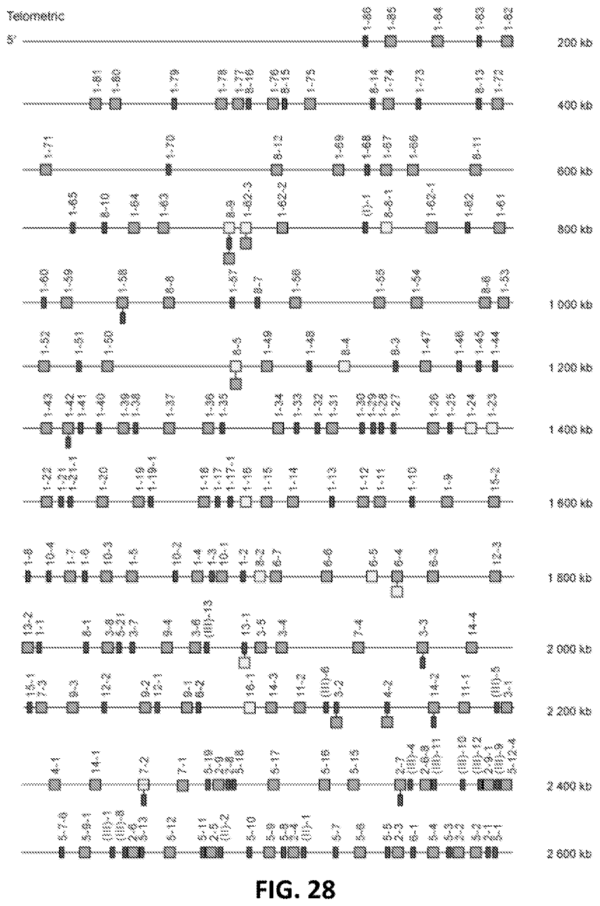

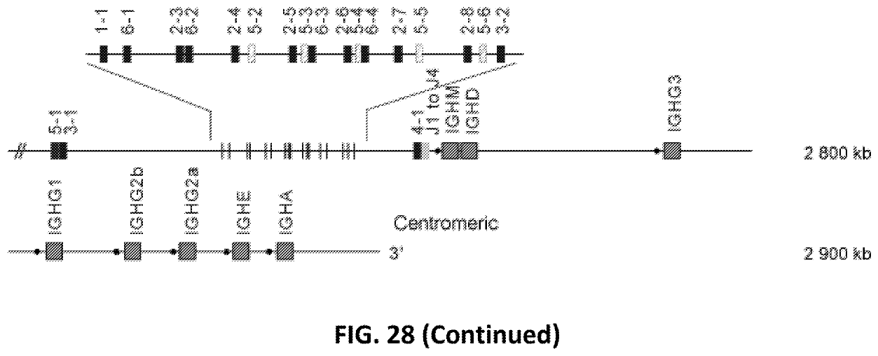

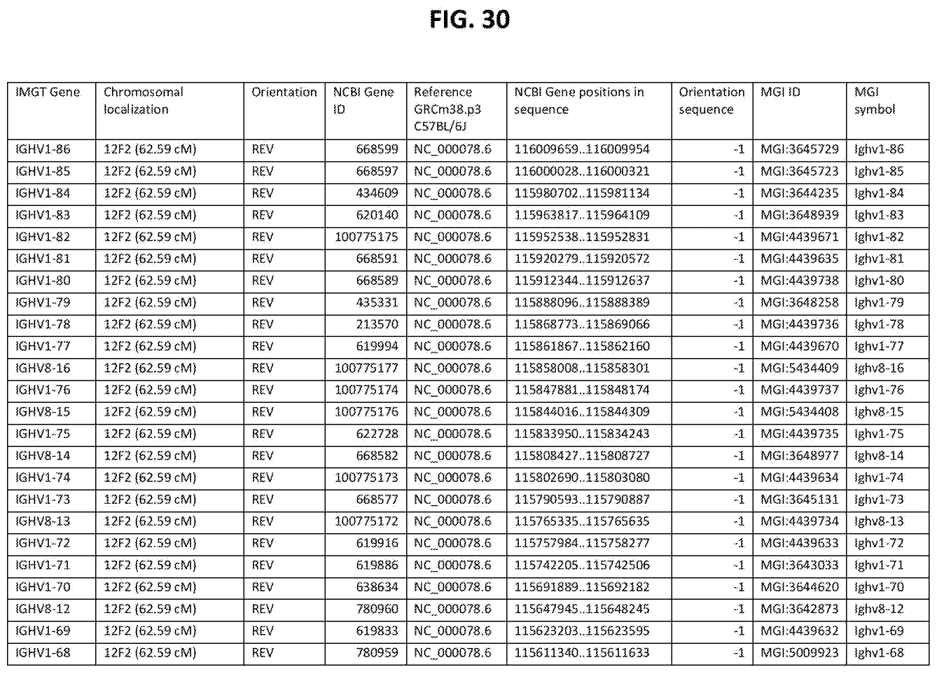

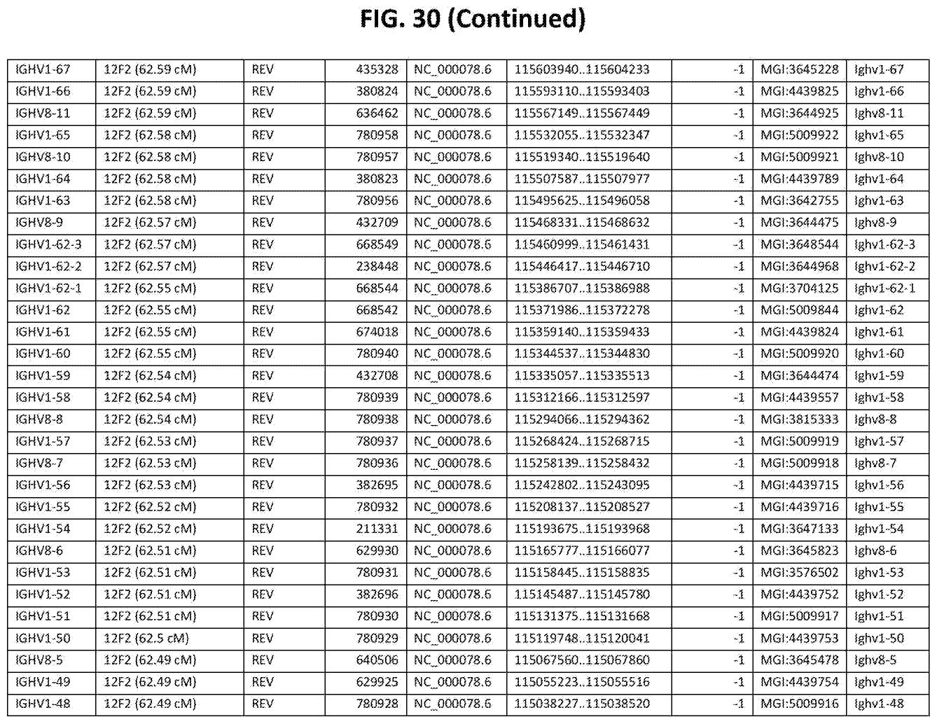

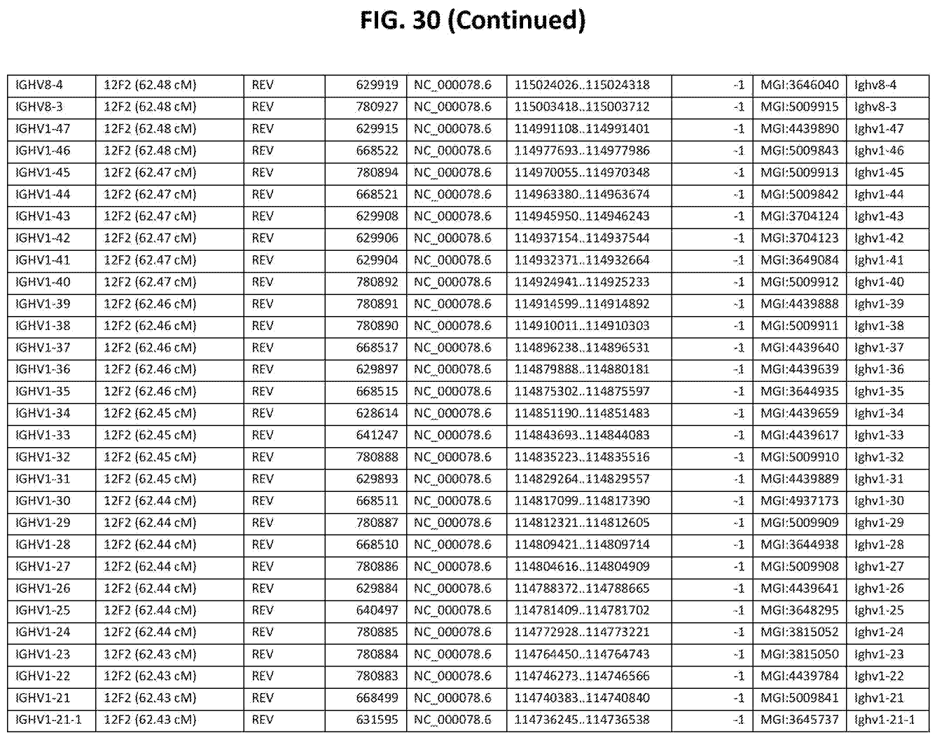

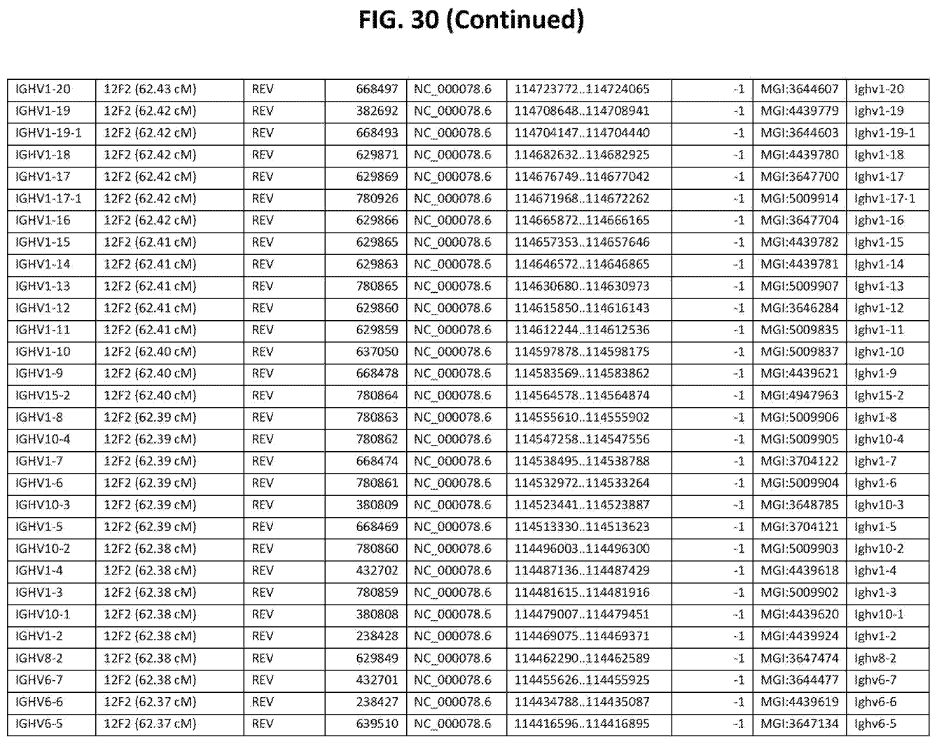

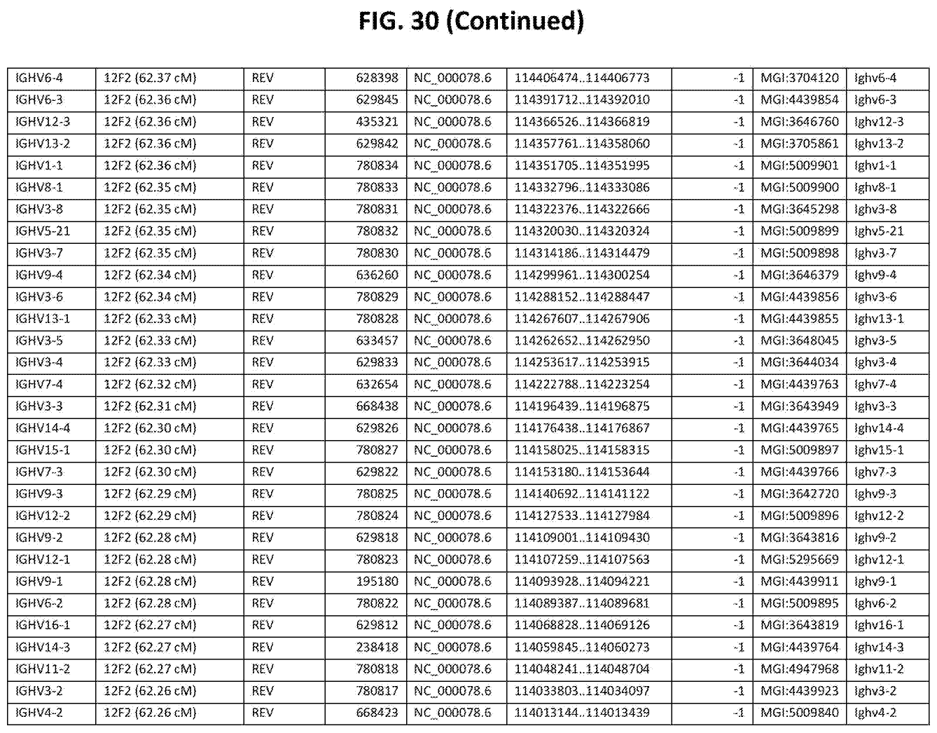

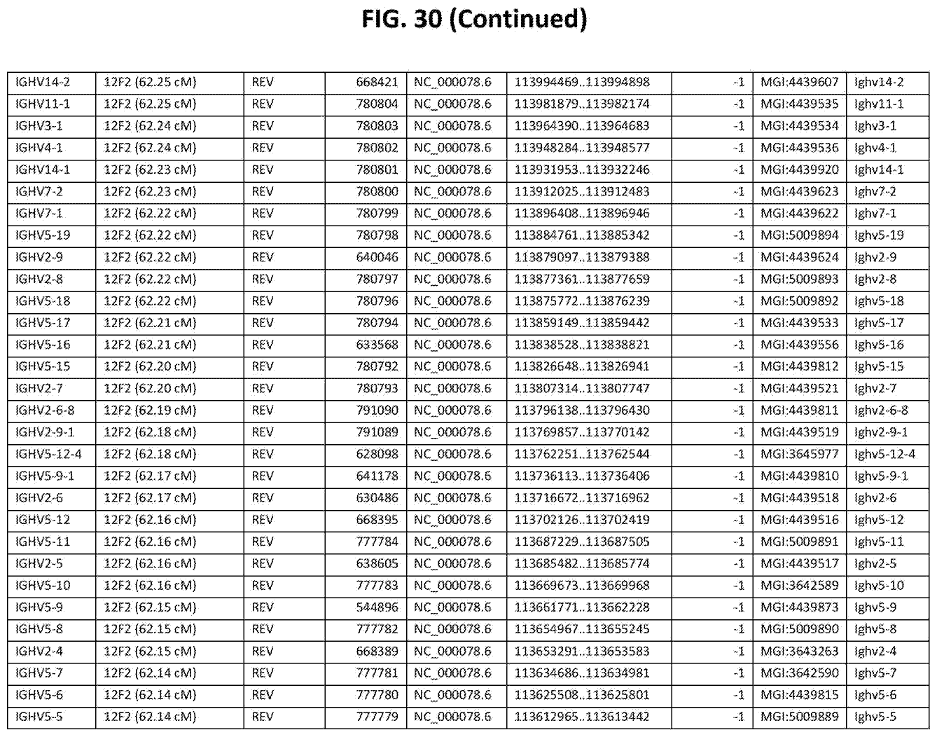

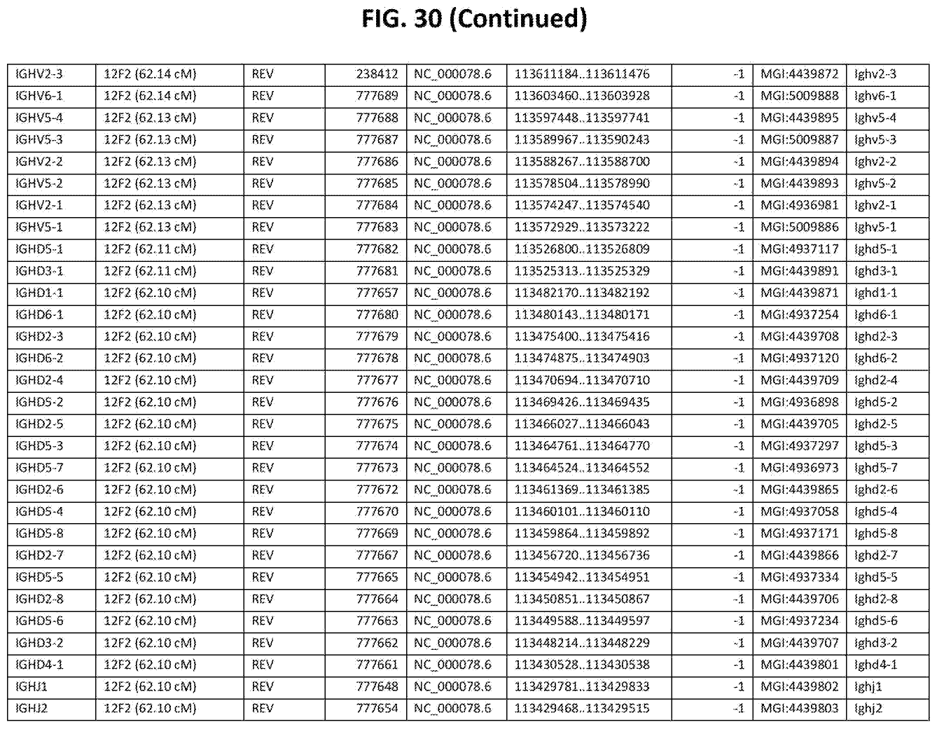

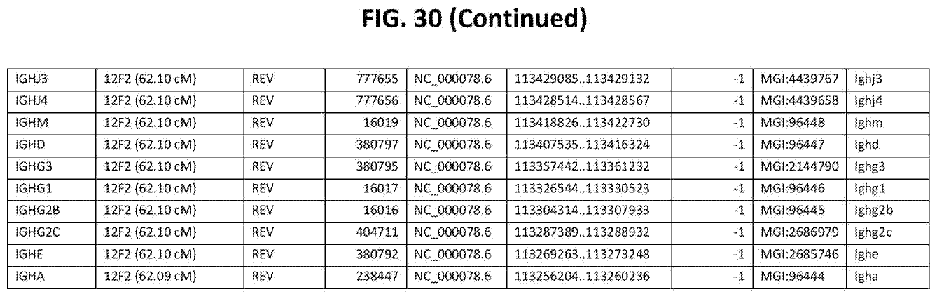

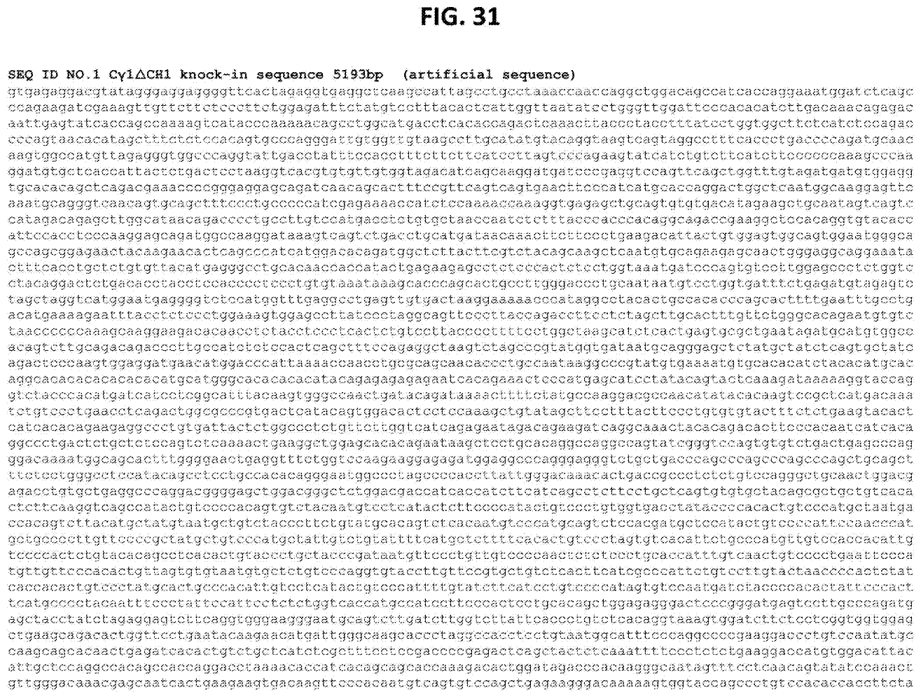

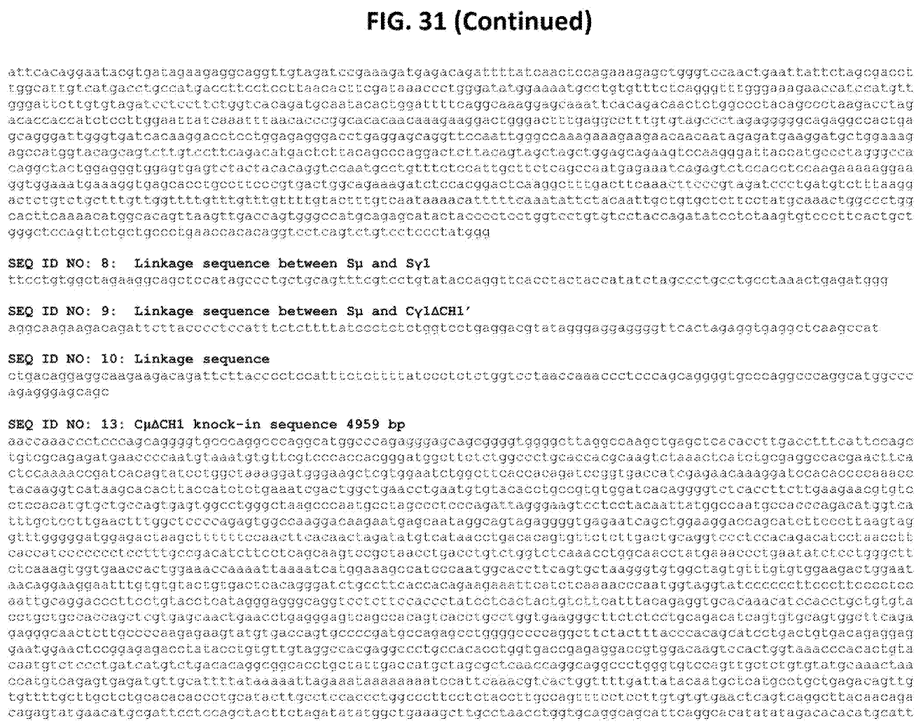

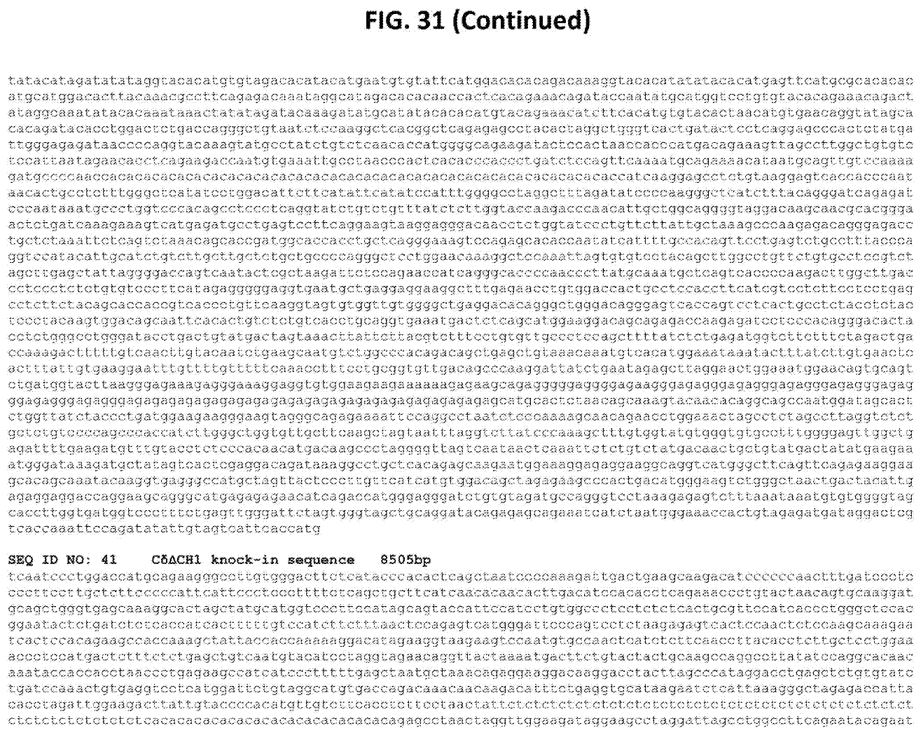

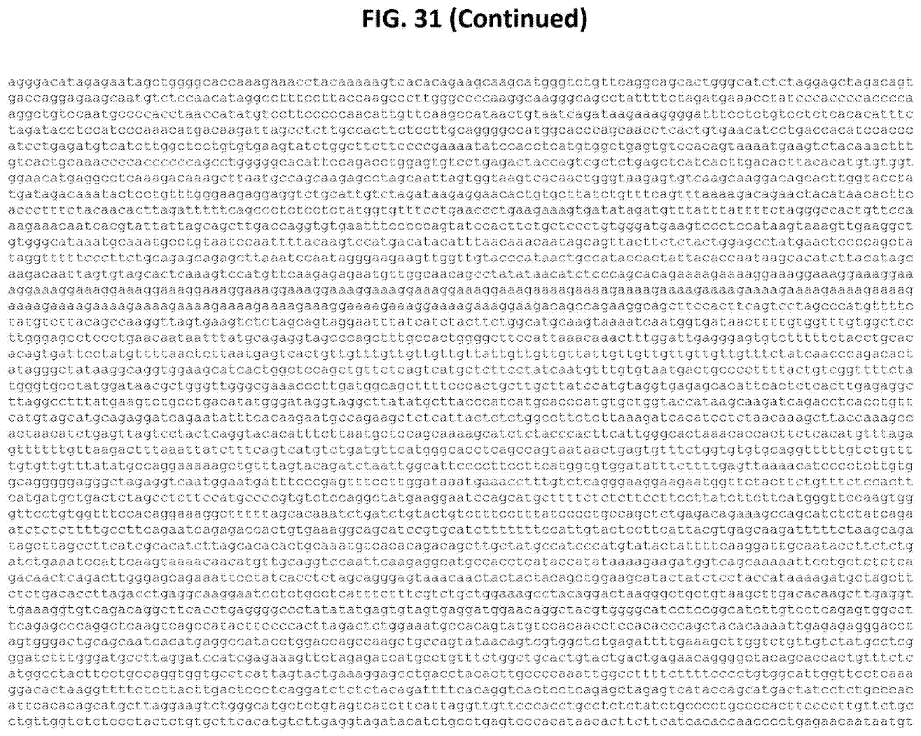

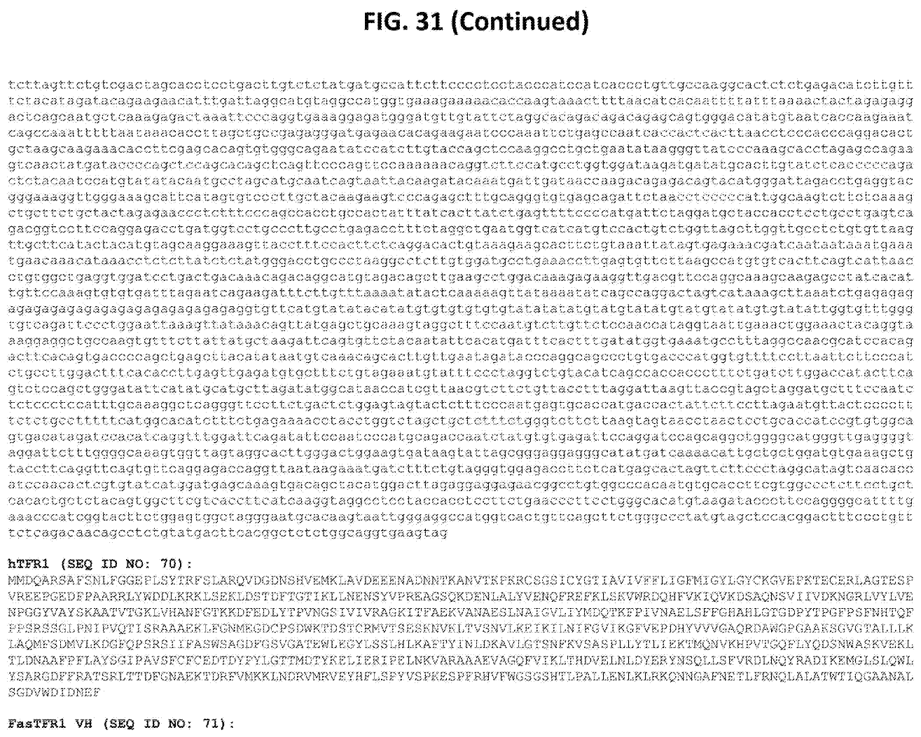

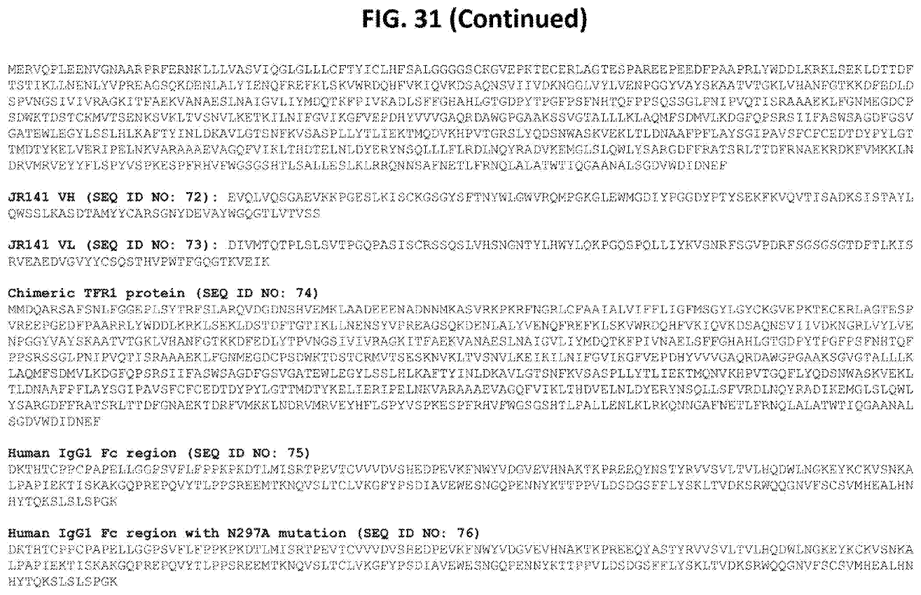

The mouse heavy chain immunoglobulin locus is located on mouse chromosome 12 (FIG. 28 and FIG. 30). Table 4 lists IGHV genes and its relative orders in this locus.

TABLE 4
List of IGHV genes on mouse chromosome 12

| Gene names | Order |
|---|---|
| IGHV1-86 | 1 |
| IGHV1-85 | 2 |
| IGHV1-84 | 3 |
| IGHV1-83 | 4 |
| IGHV1-82 | 5 |
| IGHV1-81 | 6 |
| IGHV1-80 | 7 |
| IGHV1-79 | 8 |
| IGHV1-78 | 9 |
| IGHV1-77 | 10 |
| IGHV8-16 | 11 |
| IGHV1-76 | 12 |
| IGHV8-15 | 13 |
| IGHV1-75 | 14 |
| IGHV8-14 | 15 |
| IGHV1-74 | 16 |
| IGHV1-73 | 17 |
| IGHV8-13 | 18 |
| IGHV1-72 | 19 |
| IGHV1-71 | 20 |
| IGHV1-70 | 21 |
| IGHV8-12 | 22 |
| IGHV1-69 | 23 |
| IGHV1-68 | 24 |
| IGHV1-67 | 25 |
| IGHV1-66 | 26 |
| IGHV8-11 | 27 |
| IGHV1-65 | 28 |
| IGHV8-10 | 29 |
| IGHV1-64 | 30 |
| IGHV1-63 | 31 |
| IGHV8-9 | 32 |
| IGHV1-62-3 | 33 |
| IGHV1-62-2 | 34 |
| IGHV1-62-1 | 35 |
| IGHV1-62 | 36 |
| IGHV1-61 | 37 |
| IGHV1-60 | 38 |
| IGHV1-59 | 39 |
| IGHV1-58 | 40 |
| IGHV8-8 | 41 |
| IGHV1-57 | 42 |
| IGHV8-7 | 43 |
| IGHV1-56 | 44 |
| IGHV1-55 | 45 |
| IGHV1-54 | 46 |
| IGHV8-6 | 47 |
| IGHV1-53 | 48 |
| IGHV1-52 | 49 |
| IGHV1-51 | 50 |
| IGHV1-50 | 51 |
| IGHV8-5 | 52 |
| IGHV1-49 | 53 |
| IGHV1-48 | 54 |
| IGHV8-4 | 55 |
| IGHV8-3 | 56 |
| IGHV1-47 | 57 |
| IGHV1-46 | 58 |
| IGHV1-45 | 59 |
| IGHV1-44 | 60 |
| IGHV1-43 | 61 |
| IGHV1-42 | 62 |
| IGHV1-41 | 63 |
| IGHV1-40 | 64 |
| IGHV1-39 | 65 |
| IGHV1-38 | 66 |
| IGHV1-37 | 67 |
| IGHV1-36 | 68 |
| IGHV1-35 | 69 |
| IGHV1-34 | 70 |
| IGHV1-33 | 71 |
| IGHV1-32 | 72 |
| IGHV1-31 | 73 |
| IGHV1-30 | 74 |
| IGHV1-29 | 75 |
| IGHV1-28 | 76 |
| IGHV1-27 | 77 |
| IGHV1-26 | 78 |
| IGHV1-25 | 79 |
| IGHV1-24 | 80 |
| IGHV1-23 | 81 |
| IGHV1-22 | 82 |
| IGHV1-21 | 83 |
| IGHV1-21-1 | 84 |
| IGHV1-20 | 85 |
| IGHV1-19 | 86 |
| IGHV1-19-1 | 87 |
| IGHV1-18 | 88 |
| IGHV1-17 | 89 |
| IGHV1-17-1 | 90 |
| IGHV1-16 | 91 |
| IGHV1-15 | 92 |
| IGHV1-14 | 93 |
| IGHV1-13 | 94 |
| IGHV1-12 | 95 |
| IGHV1-11 | 96 |
| IGHV1-10 | 97 |
| IGHV1-9 | 98 |
| IGHV15-2 | 99 |
| IGHV1-8 | 100 |
| IGHV10-4 | 101 |
| IGHV1-7 | 102 |
| IGHV1-6 | 103 |
| IGHV10-3 | 104 |
| IGHV1-5 | 105 |
| IGHV10-2 | 106 |
| IGHV1-4 | 107 |
| IGHV1-3 | 108 |
| IGHV10-1 | 109 |
| IGHV1-2 | 110 |
| IGHV8-2 | 111 |
| IGHV6-7 | 112 |
| IGHV6-6 | 113 |
| IGHV6-5 | 114 |
| IGHV6-4 | 115 |
| IGHV6-3 | 116 |
| IGHV12-3 | 117 |
| IGHV13-2 | 118 |
| IGHV1-1 | 119 |
| IGHV8-1 | 120 |
| IGHV3-8 | 121 |
| IGHV5-21 | 122 |
| IGHV3-7 | 123 |
| IGHV9-4 | 124 |
| IGHV3-6 | 125 |
| IGHV13-1 | 126 |
| IGHV3-5 | 127 |
| IGHV3-4 | 128 |
| IGHV7-4 | 129 |
| IGHV3-3 | 130 |
| IGHV14-4 | 131 |
| IGHV15-1 | 132 |
| IGHV7-3 | 133 |
| IGHV9-3 | 134 |
| IGHV12-2 | 135 |
| IGHV9-2 | 136 |
| IGHV12-1 | 137 |
| IGHV9-1 | 138 |
| IGHV6-2 | 139 |
| IGHV16-1 | 140 |
| IGHV14-3 | 141 |
| IGHV11-2 | 142 |
| IGHV3-2 | 143 |
| IGHV4-2 | 144 |
| IGHV14-2 | 145 |
| IGHV11-1 | 146 |
| IGHV3-1 | 147 |
| IGHV4-1 | 148 |
| IGHV14-1 | 149 |
| IGHV7-2 | 150 |
| IGHV7-1 | 151 |
| IGHV5-19 | 152 |
| IGHV2-9 | 153 |
| IGHV2-8 | 154 |
| IGHV5-18 | 155 |
| IGHV5-17 | 156 |
| IGHV5-16 | 157 |
| IGHV5-15 | 158 |
| IGHV2-7 | 159 |
| IGHV2-6-8 | 160 |
| IGHV2-9-1 | 161 |
| IGHV5-12-4 | 162 |
| IGHV5-9-1 | 163 |
| IGHV2-6 | 164 |
| IGHV5-12 | 165 |
| IGHV5-11 | 166 |
| IGHV2-5 | 167 |
| IGHV5-10 | 168 |
| IGHV5-9 | 169 |
| IGHV5-8 | 170 |
| IGHV2-4 | 171 |
| IGHV5-7 | 172 |
| IGHV5-6 | 173 |
| IGHV5-5 | 174 |
| IGHV2-3 | 175 |
| IGHV6-1 | 176 |
| IGHV5-4 | 177 |
| IGHV5-3 | 178 |
| IGHV2-2 | 179 |
| IGHV5-2 | 180 |
| IGHV2-1 | 181 |
| IGHV5-1 | 182 |

Table 5 lists all IGHD genes and its relative orders on mouse chromosome 12. Table 6 lists all IGHJ genes and its relative orders on mouse chromosome 12. The genes for immunoglobulin constant domains are after the IGHV, IGHD, and IGHJ genes. These genes include (as shown in the following order): immunoglobulin heavy constant mu (IGHM), immunoglobulin heavy constant delta (IGHδ), immunoglobulin heavy constant gamma 3 (IGHG3), immunoglobulin heavy constant gamma 1 (IGHG1), immunoglobulin heavy constant gamma 2b (IGHG2b), immunoglobulin heavy constant gamma 2c (IGHG2c), immunoglobulin heavy constant epsilon (IGHE), and immunoglobulin heavy constant alpha (IGHA) genes. In some embodiments, immunoglobulin heavy constant gamma 2a (IGHG2a) is at the position of IGHG2c. These genes and the order of these genes are also shown in FIG. 28 and FIG. 30.

TABLE 5
List of IGHD genes on mouse chromosome 12

| Gene names | Order |
|---|---|
| IGHD5-1 | 183 |
| IGHD3-1 | 184 |
| IGHD1-1 | 185 |
| IGHD6-1 | 186 |
| IGHD2-3 | 187 |
| IGHD6-2 | 188 |
| IGHD2-4 | 189 |
| IGHD2-5 | 191 |
| IGHD5-3 | 192 |
| IGHD5-7 | 193 |
| IGHD2-6 | 194 |
| IGHD5-4 | 195 |
| IGHD5-8 | 196 |
| IGHD2-7 | 197 |
| IGHD5-5 | 198 |
| IGHD2-8 | 199 |
| IGHD5-6 | 200 |
| IGHD3-2 | 201 |
| IGHD4-1 | 202 |

TABLE 6
List of IGHJ genes on mouse chromosome 12

| Gene names | Order |
|---|---|
| IGHJ1 | 203 |
| IGHJ2 | 204 |
| IGHJ3 | 205 |
| IGHJ4 | 206 |

The present disclosure provides genetically-modified, non-human animal comprising one or more human IGHV genes, one or more human IGHD genes, and/or one or more human IGHJ genes. In some embodiments, the human IGHV genes, the human IGHD genes, and the human IGHJ genes are operably linked together and can undergo VDJ rearrangement. In some embodiments, the human IGHV genes, the human IGHD genes, and the human IGHJ genes are at the endogenous heavy chain immunoglobulin gene locus.

In some embodiments, the animal compromises about or at least 1, 2, 3, 4, 5, 6, 7, 8, 9, 10, 20, 30, 40, 50, 60, 70, 80, 90, 100, 110, 120, 130, 140, 150, 151, 152, 153, 154, 155, 156, 157, 158, 159, 160 or 161 human IGHV genes (e.g., genes as shown in Table 1).

In some embodiments, the animal comprises about or at least 150, 151, 152, 153, 154, 155, 156, 157, 158, 159, 160 or 161 human IGHV genes selected from Table 1, about or at least 20, 21, 22, 23, 24, 25, 26, or 27 human IGHD genes selected from Table 2, and about or at least 5, 6, 7, 8, or 9 human IGHJ genes selected from Table 3. In some embodiments, the animal comprises all human IGHV genes in Table 1 except IGHV2-10, IGHV3-9, and IGHV1-8, all human IGHD genes in Table 2, and all human IGHJ genes in Table 3. In some embodiments, the animal comprises all human IGHV genes in Table 1 except IGHV5-10-1 and IGHV3-64D, all human IGHD genes in Table 2, and all human IGHJ genes in Table 3. In some embodiments, the animal comprises all human IGHV genes, all human IGHD genes, and all human IGHJ genes at the endogenous heavy chain immunoglobulin gene locus of human chromosome 14 of a human subject. In some embodiments, the animal comprises all human IGHV genes, all human IGHD genes, and all human IGHJ genes at the endogenous heavy chain immunoglobulin gene locus of human chromosome 14 of a human cell (e.g., a somatic cell, a cultured cell, a non-immune cell, a cell without any V (D) J rearrangement).

In some embodiments, the animal compromises 1, 2, 3, 4, 5, 6, 7, 8, 9, or 10 genes selected from IGHV(III)-82, IGHV7-81, IGHV4-80, IGHV3-79, IGHV(II)-78-1, IGHV5-78, IGHV7-77, IGHV(III)-76-1, IGHV3-76, and IGHV3-75.

In some embodiments, the animal compromises 1, 2, 3, 4, 5, 6, 7, 8, 9, or 10 genes selected from IGHV(III)-5-2, IGHV(III)-5-1, IGHV2-5, IGHV7-4-1, IGHV4-4, IGHV1-3, IGHV(III)-2-1, IGHV1-2, IGHV(II)-1-1, and IGHV6-1.

In some embodiments, the animal compromises an unmodified human sequence comprising a sequence starting from a gene selected from IGHV(III)-82, IGHV7-81, IGHV4-80, IGHV3-79, IGHV(II)-78-1, IGHV5-78, IGHV7-77, IGHV(III)-76-1, IGHV3-76, and IGHV3-75, and ending at a gene selected from IGHV(III)-5-2, IGHV(III)-5-1, IGHV2-5, IGHV7-4-1, IGHV4-4, IGHV1-3, IGHV(III)-2-1, IGHV1-2, IGHV(II)-1-1, and IGHV6-1. In some embodiments, the unmodified human sequence derived from a human heavy chain immunoglobulin gene locus starting from human IGHV(III)-82 to human IGHV1-2. In some embodiments, the unmodified human sequence derived from a human heavy chain immunoglobulin gene locus starting from human IGHV(III)-82 to human IGHV(II)-1-1. In some embodiments, the unmodified human sequence derived from a human heavy chain immunoglobulin gene locus starting from human IGHV(III)-82 to human IGHV-6-1.

In some embodiments, the animal compromises about or at least 1, 2, 3, 4, 5, 6, 7, 8, 9, 10, 11, 12, 13, 14, 15, 16, 17, 18, 19, 20, 21, 22, 23, 24, 25, 26, or 27 human IGHD genes (e.g., genes as shown in Table 2). In some embodiments, the animal compromises 1, 2, 3, 4, 5, 6, 7, 8, 9, or 10 genes selected from IGHD1-1, IGHD2-2, IGHD3-3, IGHD4-4, IGHD5-5, IGHD4-23, IGHD5-24, IGHD6-25, IGHD1-26, and IGHD7-27.

In some embodiments, the animal compromises about or at least 1, 2, 3, 4, 5, 6, 7, 8, or 9 human IGHJ genes (e.g., genes as shown in Table 3). In some embodiments, the animal compromises 1, 2, 3, 4, 5, 6, 7, 8, or 9 human IGHJ genes selected from IGHJ1P, IGHJ1, IGHJ2. IGHJ2P, IGHJ3, IGHJ4, IGHJ5, IGHJ3P, and IGHJ6.

In some embodiments, the animal compromises an unmodified human sequence comprising a sequence starting from a gene selected from IGHD1-1, IGHD2-2, IGHD3-3, IGHD4-4, IGHD5-5, IGHD4-23, IGHD5-24, IGHD6-25, IGHD1-26, and IGHD7-27, and ending at a gene selected from IGHJ1P, IGHJ1, IGHJ2, IGHJ2P, IGHJ3, IGHJ4, IGHJ5, IGHJ3P, and IGHJ6. In some embodiments, the unmodified human sequence derived from a human heavy chain immunoglobulin gene locus starting from human IGHD1-1 to human IGHJ6.

In some embodiments, the unmodified human sequence derived from a human heavy chain immunoglobulin gene locus starting from human IGHD1-1 to human IGHD7-27.

In some embodiments, the unmodified human sequence derived from a human heavy chain immunoglobulin gene locus starting from human IGHJ1P to human IGHJ6. In some embodiments, the unmodified human sequence derived from a human heavy chain immunoglobulin gene locus starting from human IGHJ1 to human IGHJ6.

In some embodiments, the unmodified human sequence derived from a human heavy chain immunoglobulin gene locus starting from human IGHV(III)-82 to human IGHJ6.

In some embodiments, the unmodified human sequence derived from a human heavy chain immunoglobulin gene locus starting from human IGHV1-2 to human IGHJ6. In some embodiments, the unmodified human sequence derived from a human heavy chain immunoglobulin gene locus starting from human IGHV(II)-1-1 to human IGHJ6. In some embodiments, the unmodified human sequence derived from a human heavy chain immunoglobulin gene locus starting from human IGHV6-1 to human IGHJ6.

In some embodiments, the animal can have one, two, three, four, five, six, seven, eight, nine, or ten unmodified human sequences. In some embodiments, the unmodified human sequence has a length of about or at least 10, 20, 30, 40, 50, 60, 70, 80, 90, 100, 200, 300, 400, 500, 600, 700, 800, 900, or 1000 kb.

In some aspects, the disclosure relates to a genetically-modified animal comprising at an endogenous heavy chain immunoglobulin gene locus, a first sequence comprising one or more human IGHV genes; a second sequence comprising an endogenous sequence; and a third sequence comprising one or more human IGHD genes, and one or more human IGHJ genes, wherein the first sequence, the second sequence, and the third sequence are operably linked.

In some embodiments, the first sequence comprises about or at least 150, 151, 152, 153, 154, 155, 156, 157, 158, 159, 160 or 161 human IGHV genes selected from Table 1. In some embodiments, the first sequence comprises about or at least 20, 21, 22, 23, 24, 25, 26, or 27 human IGHD genes selected from Table 2.

In some embodiments, the first sequence is an unmodified sequence derived from a human heavy chain immunoglobulin gene locus. In some embodiments, the first sequence is about or at least 10, 20, 30, 40, 50, 60, 70, 80, 90, 100, 200, 300, 400, 500, 600, 700, 800, 900, or 1000 kb.

In some embodiments, the second sequence comprises an endogenous sequence that is about or at least 1, 2, 3, 4, 5, 6, 7, 8, 9, 10, 11, 12, 13, 14, 15, 16, 17, 18, 19, or 20 kb.

In some embodiments, the third sequence comprises about or at least 20, 21, 22, 23, 24, 25, 26, or 27 human IGHD genes selected from Table 2. In some embodiments, the third sequence comprises about or at least 5, 6, 7, 8, or 9 human IGHJ genes selected from Table 3. In some embodiments, the third sequence comprises all human IGHD genes in Table 2, and all human IGHJ genes in Table 3.

In some embodiments, the animal comprises one or more endogenous genes selected from the group consisting of immunoglobulin heavy constant mu (IGHM), immunoglobulin heavy constant delta (IGHδ), immunoglobulin heavy constant gamma 3 (IGHG3), immunoglobulin heavy constant gamma 1 (IGHG1), immunoglobulin heavy constant gamma 2b (IGHG2b), immunoglobulin heavy constant gamma 2c (IGHG2c), immunoglobulin heavy constant epsilon (IGHE), and immunoglobulin heavy constant alpha (IGHA) genes. In some embodiments, immunoglobulin heavy constant gamma 2a (IGHG2a) is at the position of IGHG2c. In some embodiments, these endogenous genes are operably linked together. In some embodiments, these endogenous genes have the same order as in a wildtype animal. In some embodiments, isotype switching (immunoglobulin class switching) can occur in the animal.

In some embodiments, the IGHV genes, the IGHD genes, and/or the IGHJ genes are operably linked together. The VDJ recombination can occur among these genes and produce functional antibodies. In some embodiments, these genes are arranged in an order that is similar to the order in human heavy chain immunoglobulin locus. This arrangement offers various advantages, e.g., the arrangement of these genes allow the production of heavy chain variable domains with a diversity that is very similar to the diversity of the heavy chain variable domains in human. As some random sequences may be inserted to the sequence during VDJ recombination, in some embodiments, the complete human antibody repertoires with no or minimum modifications can reduce the likelihood that non-human sequence is inserted during the VDJ recombination.

In some embodiments, the IGHV genes, the IGHD genes, and/or the IGHJ genes are operably linked together to one or more genes (e.g., all genes) selected from IGHM, IGHδ, IGHG3, IGHG1, IGHG2a, IGHG2b, IGHG2c. IGHE, and IGHA genes.

In some embodiments, the animal comprises a disruption in the animal's endogenous heavy chain immunoglobulin gene locus. In some embodiments, the disruption in the animal's endogenous heavy chain immunoglobulin gene locus comprises a deletion of one or more endogenous IGHV genes, one or more endogenous IGHD genes, and one or more endogenous IGHJ genes.

In some embodiments, the animal is a mouse. The disruption in the animal's endogenous heavy chain immunoglobulin gene locus comprises a deletion of at least or about 1, 2, 3, 4, 5, 6, 7, 8, 9, 10, 20, 30, 40, 50, 60, 70, 80, 90, 100, 110, 120, 130, 140, 150, 160, 170, 171, 172, 173, 174, 175, 176, 177, 178, 179, 180, 181, or 182 mouse IGHV genes (e.g., genes as shown in Table 4). In some embodiments, the disruption compromises a deletion of about or at least 1, 2, 3, 4, 5, 6, 7, 8, 9, or 10 mouse IGHV genes selected from IGHV1-86, IGHV1-85, IGHV1-84, IGHV1-83, IGHV1-82, IGHV1-81, IGHV1-80, IGHV1-79, IGHV1-78, and IGHV1-77. In some embodiments, the mouse still compromises about or at least 1, 2, 3, 4, 5, 6, 7, 8, 9, or 10 mouse IGHV genes selected from IGHV1-86, IGHV1-85, IGHV1-84, IGHV1-83, IGHV1-82, IGHV1-81, IGHV1-80, IGHV1-79, IGHV1-78, and IGHV1-77 (e.g., IGHV1-86).

In some embodiments, the disruption compromises a deletion of about or at least 1, 2, 3, 4, 5, 6, 7, 8, 9, or 10 mouse IGHV genes selected from IGHV5-6, IGHV5-5, IGHV2-3, IGHV6-1, IGHV5-4, IGHV5-3, IGHV2-2, IGHV5-2, IGHV2-1, and IGHV5-1. In some embodiments, the mouse still compromises a deletion of about or at least 1, 2, 3, 4, 5, 6, 7, 8, 9, or 10 mouse IGHV genes selected from IGHV5-6, IGHV5-5, IGHV2-3, IGHV6-1, IGHV5-4, IGHV5-3, IGHV2-2, IGHV5-2, IGHV2-1, and IGHV5-1.

In some embodiments, the disruption in the animal's endogenous heavy chain immunoglobulin gene locus comprises a deletion of at least or about 1, 2, 3, 4, 5, 6, 7, 8, 9, 10, 11, 12, 13, 14, 15, 16, 17, 18, 19, or 20 mouse IGHD genes (e.g., genes as shown in Table 5). In some embodiments, the disruption compromises a deletion of about or at least 1, 2, 3, 4, 5, 6, 7, 8, 9, or 10 mouse IGHD genes selected from IGHD5-1, IGHD3-1, IGHD1-1, IGHD6-1, IGHD2-3, IGHD2-7, IGHD2-8, IGHD5-6, IGHD3-2, and IGHD4-1. In some embodiments, the mouse still compromises about or at least 1, 2, 3, 4, 5, 6, 7, 8, 9, or 10 mouse IGHD) genes selected from IGHD5-1, IGHD3-1, IGHD1-1, IGHD6-1, IGHD2-3, IGHD2-7, IGHD2-8, IGHD5-6, IGHD3-2, and IGHD4-1.

In some embodiments, the disruption compromises a deletion of about or at least 1, 2, 3, or 4 mouse IGHJ genes selected from IGHJ1, IGHJ2, IGHJ3, and IGHJ4. In some embodiments, the mouse still compromises about or at least 1, 2, 3, or 4 mouse IGHJ genes selected from IGHJ1, IGHJ2, IGHJ3, and IGHJ4.

In some embodiments, the disruption in the animal's endogenous heavy chain immunoglobulin gene locus comprises a deletion of about or at least 500 kb, 600 kb, 700 kb, 800 kb, 900 kb, 1000 kb, 1500 kb, 2000 kb, 2500 kb, or 3000 kb of an endogenous sequence.

In some embodiments, the deleted sequence starts from IGHV1-86 to IGHJ4, from IGHV1-85 to IGHJ4, from IGHV1-84 to IGHJ4, from IGHV1-83 to IGHJ4, or from IGHV1-82 to IGHJ4 (e.g., from IGHV1-85 to IGHJ4).

In some embodiments, the animal comprises about or at least 1, 2, 3, 4, 5, 6, 7, 8, 9, or 10 sequences that are at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a sequence in the human heavy chain immunoglobulin gene locus. In some embodiments, the sequence has a length of about or at least 10, 20, 30, 40, 50, 60, 70, 80, 90, 100, 200, 300, 400, 500, 600, 700, 800, 900, 1000, 1500, 2000, 2500, 3000 or 3500 kb. In some embodiments, the sequence starts from human IGHV(III)-82 to IGHV1-2. In some embodiments, the sequence starts from human IGHV7-81 to IGHV1-2. In some embodiments, the sequence starts from human IGHV(II)-1-1 to IGHVJ6. In some embodiments, the sequence starts from human IGHV6-1 to IGHVJ6.

The human IGHV genes, the human IGHD genes, and the human IGHJ genes are operably linked together and can undergo VDJ rearrangement. In some embodiments, the modified mouse has complete human IGHV, IGHD, and IGHJ gene repertoires (e.g., including all non-pseudo human IGHV, IGHD, and IGHJ genes). Thus, the modified mouse can produce a complete human antibody repertory. In some embodiments, after VDJ recombination, one IGHV gene (e.g., IGHV3-21 or IGHV3-74) contributes to the sequence that encodes an antibody heavy chain variable region. One IGHD gene contributes to the sequence that encodes an antibody heavy chain variable region. And one IGHJ gene contributes to the sequence that encodes an antibody heavy chain variable region. In some embodiments, the IGHV gene is IGHV3-21 or IGHV3-74.

In some embodiments, one IGHV gene (e.g., IGHV3-30, IGHV3-33, IGHV4-39, or IGHV4-34) contributes to the sequence that encodes an antibody heavy chain variable region. One IGHD gene (e.g., IGHD6-19) contributes to the sequence that encodes an antibody heavy chain variable region. And one IGHJ gene (e.g., IGHJ4 or IGHJ6) contributes to the sequence that encodes an antibody heavy chain variable region.

Furthermore, in some cases, the entire mouse IGHV genes, IGHD genes, and IGHJ genes (e.g., including all none-pseudo genes) are knocked out, and the heavy chain variable region will not have any sequence that is encoded by a sequence derived from the mouse, thereby minimizing immunogenicity in human.

In some embodiments, the locus can have about or at least 1, 2, 3, 4, 5, 6, 7, 8, 9, 10, 12, 14, 16, 18, 20, 30 or 40 non-human exogenous IGHV genes (e.g., from camelid). In some embodiments, the locus can have about or at least 1, 2, 3, 4, 5, 6, 7, 8, 9, 10, 12, 14, 16, 18, 20, 30 or 40 non-human exogenous IGHD genes (e.g., from camelid). In some embodiments, the locus can have about or at least 1, 2, 3, 4, 5, 6, 7, 8, 9, 10, 12, 14, 16, 18, 20, 30 or 40 non-human exogenous IGHJ genes (e.g., from camelid). These non-human exogenous genes can help improve the diversity of VHH domains.

Various modifications on the heavy chain immunoglobulin locus are described e.g., in WO2020169022A1, which is incorporated herein by reference in its entirety.

### Immunoglobulin Light Chain Locus

In some embodiments, the animal has an intact kappa and/or lambda chain immunoglobulin locus. In some embodiments, the animal has a disrupted kappa and/or lambda chain immunoglobulin locus.

Kappa chain immunoglobulin locus (also known as IGK or immunoglobulin kappa locus) is a region on the chromosome (e.g., chromosome 6) that contains genes for the light chains of human antibodies (or immunoglobulins). Similarly, the immunoglobulin light chain genes can also undergo a series rearrangement that lead to the production of a mature immunoglobulin light-chain nucleic acid (e.g., a kappa chain).

The joining of a V segment (also known as an IGKV gene) and a J segment (also known as an IGKJ gene) creates a continuous exon that encodes the whole of the light-chain variable domain. In the unrearranged DNA, the V gene segments (or IGKV gene cluster) are located relatively far away from the C region. The J gene segments (or IGKJ gene cluster) are located close to the C region. Joining of a V segment to a J gene segment also brings the V gene close to a C-region sequence. The J gene segment of the rearranged V region is separated from a C-region sequence only by an intron. To make a complete immunoglobulin light-chain messenger RNA, the V-region exon is joined to the C-region sequence by RNA splicing after transcription.

In some embodiments, the animal comprises a disruption in the animal's endogenous light chain immunoglobulin gene locus. In some embodiments, the disruption in the animal's endogenous light chain immunoglobulin gene locus comprises a deletion of one or more endogenous IGKV genes, and one or more endogenous IGKJ genes.

In some embodiments, the animal is a mouse. The disruption in the animal's endogenous kappa chain immunoglobulin gene locus comprises a deletion of at least or about 1, 2, 3, 4, 5, 6, 7, 8, 9, 10, 20, 30, 40, 50, 60, 70, 80, 90, 100, 110, 120, 130, 140, 150, 151, 152, 153, 154, 155, 156, 157, 158, 159, 160, 161, 162, or 163 mouse IGKV genes. In some embodiments, the disruption compromises a deletion of about or at least 1, 2, 3, 4, 5, 6, 7, 8, 9, or 10 mouse IGKV genes selected from IGKV2-137, IGKV1-136, IGKV1-135, IGKV14-134-1, IGKV17-134, IGKV1-133, IGKV1-132, IGKV1-131, IGKV14-130, and IGKV9-129. In some embodiments, the mouse still compromises about or at least 1, 2, 3, 4, 5, 6, 7, 8, 9, or 10 mouse IGKV genes selected from IGKV2-137, IGKV1-136, IGKV1-135, IGKV14-134-1, IGKV17-134, IGKV1-133, IGKV1-132, IGKV1-131, IGKV14-130, and IGKV9-129.

In some embodiments, the disruption compromises a deletion of about or at least 1, 2, 3, 4, 5, 6, 7, 8, 9, or 10 mouse IGKV genes selected from IGKV3-10, IGKV3-9, IGKV3-8, IGKV3-7, IGKV3-6, IGKV3-5, IGKV3-4, IGKV3-3, IGKV3-2, and IGKV3-1. In some embodiments, the mouse still compromises about or at least 1, 2, 3, 4, 5, 6, 7, 8, 9, or 10 mouse IGKV genes selected from IGKV3-10, IGKV3-9, IGKV3-8, IGKV3-7, IGKV3-6, IGKV3-5, IGKV3-4, IGKV3-3, IGKV3-2, and IGKV3-1.

In some embodiments, the disruption compromises a deletion of about or at least 1, 2, 3, 4, or 5 mouse IGKJ genes selected from IGKJ1, IGKJ2, IGKJ3, IGKJ4, and IGKJ5. In some embodiments, the mouse still compromises about or at least 1, 2, 3, 4, or 5 mouse IGKJ genes selected from IGKJ1, IGKJ2, IGKJ3, IGKJ4, and IGKJ5 (e.g., IGKJ5).

In some embodiments, the disruption in the animal's endogenous kappa light chain immunoglobulin gene locus comprises a deletion of about or at least 500 kb, 600 kb, 700 kb, 800 kb, 900 kb, 1000 kb, 1500 kb, 2000 kb, 2500 kb, 3000 kb or 3500 kb of an endogenous sequence.

In some embodiments, the deleted sequence starts from IGKV2-137 to IGKJ4, from IGKV1-136 to IGKJ4, from IGKV1-135 to IGKJ4, from IGKV2-137 to IGKJ5, from IGKV1-136 to IGKJ5, or from IGKV1-135 to IGKJ5 (e.g., from IGKV2-137 to IGKJ5).

In some embodiments, the animal comprises a disruption in the animal's endogenous lambda light chain immunoglobulin gene locus. In some embodiments, the disruption in the animal's endogenous light chain immunoglobulin gene locus comprises a deletion of one or more endogenous IGLV genes, one or more endogenous IGLJ genes, and/or one or more immunoglobulin lambda constant (IGLC) genes (e.g., IGLC1, IGLC2, IGLC3, and IGLC4).

The disruption in the animal's endogenous lambda light chain immunoglobulin gene locus comprises a deletion of at least or about 1, 2, 3, 4, 5, 6, 7, 8, 9, 10, 11, or 12 mouse IGLV, IGLJ, and IGLC genes. In some embodiments, the deletion compromises about or at least 1, 2, 3, or 4 mouse IGKC genes selected from IGLC1, IGLC2, IGLC3, and IGLC4. In some embodiments, the disruption compromises a deletion of about or at least 1, 2, or 3 mouse IGLV genes selected from IGLV1, IGLV2, and IGLV3. In some embodiments, the disruption compromises a deletion of about or at least 1, 2, 3, 4, or 5 mouse IGLJ genes selected from IGLJ1, IGLJ2, IGLJ3, IGLJ3P, and IGLJ4.

In some embodiments, the disruption in the animal's endogenous lambda light chain immunoglobulin gene locus comprises a deletion of about or at least 10 kb, 20 kb, 30 kb, 40 kb, 50 kb, 60 kb, 70 kb, 80 kb, 90 kb, 100 kb, 110 kb, 120 kb, 130 kb, 140 kb, 150 kb, 160 kb, 170 kb, 180 kb, 190 kb, 200 kb, 210 kb, 220 kb, 230 kb, 240 kb, 250 kb, 260 kb, 270 kb, 280 kb, 290 kb, 300 kb, 350 kb, 400 kb, 450 kb, 500 kb, or 1000 kb of nucleotides. In some embodiments, there is no disruption in the animal's endogenous lambda light chain immunoglobulin gene.

In some embodiments, the deleted sequence starts from IGLV2 to IGLC1, from IGLV3 to IGLC1, or from IGLJ2 to IGLC1.

Various modifications on the light chain immunoglobulin locus are described e.g., in WO2020169022A1, which is incorporated herein by reference in its entirety.

### Genetically Modified Animals

The present application provides genetically modified non-human animals that produce heavy chain-only antibodies, i.e., antibodies that lack light chains. In some embodiments, the genetically modified non-human animal does not produce IgG (e.g., IgG1) molecules comprising light chains. In some embodiments, the genetically modified non-human animal does not produce any conventional IgG molecules, i.e., IgG antibodies that have two heavy chains and two light chains.

In some embodiments, the genetically modified non-human animal has a fully functional endogenous light chain locus. In some embodiments, the immunoglobulin heavy chain locus of the animal comprises a fully functional IGHM, IGHδ, and/or IGHA gene. In some embodiments, the genome of the animal does not comprise exogenous sequences (e.g., human immunoglobulin heavy chain constant region genes) in the endogenous immunoglobulin heavy chain constant region locus.

In some embodiments, the modified immunoglobulin heavy chain locus lacks the CH1 exon of endogenous IGHG gene (e.g., IGHG1 gene). In some embodiments, the immunoglobulin heavy chain locus of the animal comprises fully functional H, CH2, CH3, M1, and/or M2 exons of IGHG1 gene.

In some embodiments, the immunoglobulin heavy chain locus of the animal only has modifications to the CH1 exon of endogenous IGHG1 gene. In some embodiments, the modification does not comprise a mutation, such as deletion or loss-of-function mutation, of a gene encoding a hinge region of the IgG1. In some embodiments, the modification does not comprise a mutation, such as deletion or loss-of-function mutation to the CH2 or the CH3 exons of the IGHG1 gene. In some embodiments, the immunoglobulin heavy chain locus does not have mutations or modifications to the CH1 exon of IGHM gene. In some embodiments, the genetically modified non-human animal has a functional gene segment encoding the CH1 domain of IgM at the endogenous IGHM locus.

In some embodiments, the genetically modified non-human animal has fully functional genes encoding other heavy chain constant isotypes, such as IgM, IgD, and/or IgA. In some embodiments, the genetically modified non-human animal has fully functional IGHM, IGHδ and/or IGHA genes. In some embodiments, the modified immunoglobulin heavy chain locus has wild-type IGHM, IGHδ, and/or IGHA genes. In some embodiments, the modified immunoglobulin heavy chain locus does not comprise any mutation to the endogenous IGHM, IGHδ and/or IGHA genes. In some embodiments, the endogenous immunoglobulin heavy chain locus has an intact endogenous IGHM, IGHδ, or IGHA gene. In some embodiments, the genetically modified non-human animal expresses wild-type IgM, IgD, and/or IgA proteins.

In some embodiments, the immunoglobulin heavy chain locus includes a mutation (e.g., deletion) or modifications to the CH1 exon of IGHM and/or IGHδ genes. In some embodiments, the endogenous immunoglobulin heavy chain locus has a modified IGHM gene and/or a modified IGHδ gene. In some embodiments, the genetically modified non-human animal does not express wild-type IgM and/or IgD. In some embodiments, the genetically modified non-human animal expresses IgM lacking a CH1 domain and/or IgD lacking a CH1 domain.

In some embodiments, introduction of modifications to the endogenous immunoglobulin heavy chain locus in the genetically modified non-human animal maintain health of the animal, including substantially normal B cell development and maturation. In some embodiments, introduction of modifications to the endogenous immunoglobulin heavy chain locus in the genetically modified non-human animal reduces or avoids immunogenicity of exogenous sequences. In some embodiments, introduction of minimal changes to the endogenous immunoglobulin heavy chain locus in the genetically modified non-human animal preserves the normal functions of the endogenous immunoglobulin heavy chain locus, including V-D-J recombination, classic switch recombination, and somatic hyper-mutation.

In some embodiments, the genetically modified non-human animal has one or more fully functional light chain loci, e.g., lambda light chain locus, and/or kappa light chain locus. In some embodiments, the genetically modified non-human animal has an unaltered endogenous light chain locus. In some embodiments, no mutations are introduced to the endogenous light chain loci of the genetically modified non-human animal. In some embodiments, the lambda and/or kappa light chain variable region locus of the genetically modified non-human animal is functional, not silenced. In some embodiments, the genetically modified non-human animal expresses a wild-type lambda light chain, and/or wild-type kappa light chain. In some embodiments, the genetically modified non-human animal expresses functional IgM molecules comprising light chains. In some embodiments, the genetically modified non-human animal expresses functional IgA, IgD, and/or IgM molecules comprising light chains. In some embodiments, the genetically modified non-human animal does not have an exogenous light chain gene or gene cluster. For example, the lambda and/or kappa light chain variable region locus of the genetically modified non-human animal can be knocked out.

In some embodiments, the modified immunoglobulin heavy chain locus does not comprises rearranged genes (e.g., rearranged IGHV, IGHD, and/or IGHJ genes). In some embodiments, the modified immunoglobulin heavy chain locus comprises the full set of unrearranged human IGHV, IGHD, and IGHJ genes.

In some embodiments, the modified immunoglobulin heavy chain locus comprises a functional splice site immediately after the CH1 exon of endogenous IGHG1 gene. In some embodiments, the modified immunoglobulin heavy chain locus comprises a wild-type splice site immediately after the CH1 exon of endogenous IGHG1 gene

In some embodiments, the animal described herein expresses a membrane-bound IgG1 lacking a CH1 domain. In some embodiments, the animal described herein expresses a soluble IgG1 lacking a CH1 domain.

In one aspect, the present disclosure provides genetically-modified, non-human animal comprising a humanized heavy chain immunoglobulin locus. In some embodiments, the animal comprises one or more human IGHV genes, one or more human IGHD genes, and/or one or more human IGHJ genes. In some embodiments, these genes are at the endogenous immunoglobulin gene locus.

In some embodiments, the animal comprises an endogenous kappa or lambda chain immunoglobulin locus. In some embodiments, the animal does not comprise an endogenous kappa or lambda chain immunoglobulin locus. In some embodiments, the animal comprises a disruption in the animal's endogenous kappa or lambda light chain immunoglobulin gene locus. In some embodiments, the animal does not have a disruption in the animal's endogenous kappa or lambda light chain immunoglobulin gene locus.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety. Thus, in various embodiments, human V, D, and/or J segments can be operably linked to non-human animal (e.g., rodent, mouse, rat, hamster) constant region gene sequences. During B cell development, these rearranged human V, D, and/or J segments are linked to the non-human animal immunoglobulin constant region.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129Sγ, 129S8, 129T1, 129T2. These mice are described, e.g., in Festing et al., Revised nomenclature for strain 129 mice, Mammalian Genome 10:836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129). In some embodiments, the non-human animal is a rodent. In some embodiments, the non-human animal is a mouse having a BALB/c, A, A/Hc, A/J, A/WySN, AKR, AKR/A, AKR/J, AKR/N, TAI, TA2, RF, SWR, C3H, C57BR, SJL, C57L, DBA/2, KM, NIH, ICR, CFW, FACA, C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL (C57BL/10Cr and C57BL/Ola), C58, CBA/Br, CBA/Cα, CBA/J, CBA/st, or CBA/H background.

Different animals have different germline organization and genes at their endogenous immunoglobulin heavy chain (IgH) loci. The IgH loci of many species have been sequenced. The gene positions and exon/intron organization of the IgH loci in mouse, rat and rabbit can be found, for example, at IMGT repertoire and NCBI databases, which are incorporated herein by reference in the entirety.

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the humanized animal is made.

Genetically modified non-human animals that comprise a modification of an endogenous non-human immunoglobulin gene locus. In some embodiments, the modification can comprise a human nucleic acid sequence encoding at least a portion of a human protein (e.g., at least 10%, 20%, 30%, 40%, 50%, 60%, 70%, 80%, 90%, 95%, 96%, 97%, 98%, or 99% identical to the human heavy chain variable domain or light chain variable domain sequence). Although genetically modified cells are also provided that can comprise the modifications described herein (e.g., ES cells, somatic cells), in many embodiments, the genetically modified non-human animals comprise the modification of the endogenous locus in the germline of the animal.

Genetically modified animals can express a humanized antibody and/or a chimeric antibody from endogenous mouse loci, wherein one or more endogenous mouse immunoglobulin genes have been replaced with human immunoglobulin genes and/or a nucleotide sequence that is at least 10%, 20%, 30%, 40%, 50%, 60%, 70%, 80%, 90%, 95%, 96%, 97%, 98%, or 99% identical to the human immunoglobulin gene sequences (e.g., IGHV, IGHD, IGHJ, IGKV and/or IGKJ genes). In various embodiments, an endogenous non-human immunoglobulin gene locus is modified in whole or in part to comprise human nucleic acid sequence.

Genetic, molecular and behavioral analyses for the non-human mammals described above can performed. The present disclosure also relates to the progeny produced by the non-human mammal provided by the present disclosure mated with the same or other genotypes. Non-human mammals can be any non-human animal known in the art and which can be used in the methods as described herein. Preferred non-human mammals are mammals, (e.g., rodents). In some embodiments, the non-human mammal is a mouse.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cell can be obtained from the cell culture established using the same constructs and the standard cell transfection techniques. The integration of genetic constructs containing DNA sequences encoding human or humanized immunoglobulins can be detected by a variety of methods.

There are many analytical methods that can be used to detect exogenous DNA or modifications on the genomic DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized proteins.

### Antibodies and Antigen Binding Fragments

The present disclosure provides antibodies and antigen-binding fragments thereof (e.g., heavy chain antibodies, humanized heavy chain antibodies, or multi-specific antibodies) that are produced by the methods described herein.

In general, conventional antibodies are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or subclasses including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, V_{H}) and multiple constant domains (or constant regions), bind to one another via disulfide bonding within their constant domains to form the “stem” of the antibody. The light chains, which each contain one variable domain (or variable region, V_{L}) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132:211-250; Martin et al., Methods Enzymol. 203:121-53 (1991); Morea et al., Biophys Chem. 68 (1-3): 9-16 (October 1997); Morea et al., J Mol Biol. 275 (2): 269-94 (January 1998); Chothia et al., Nature 342 (6252): 877-83 (December 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG2c, IgG3, IgG4, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, cd. The human IgG subclasses: molecular analysis of structure, function and regulation. Elsevier, 2016; each of which is incorporated herein by reference in its entirety. The heavy chain constant regions in the heavy chain antibodies can be derived from any immunoglobulin molecules described herein (e.g., IgG1, IgG2a, IgG2b, IgG2c, IgG3, IgG4, IgM, IgD, IgE, IgA).

The antibody can also be an immunoglobulin molecule that is derived from any species (e.g., human, rodent, mouse, rat, camelid). Antibodies disclosed herein also include, but are not limited to, polyclonal, monoclonal, monospecific, multi-specific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antigen binding fragment can form a part of a chimeric antigen receptor (CAR). In some embodiments, the chimeric antigen receptor are fusions of VHH as described herein, fused to CD3-zeta transmembrane- and endodomain.

The antibodies and antigen-binding fragments thereof (e.g., humanized antibodies or chimeric antibodies) that are produced by the methods described herein have various advantages. In some embodiments, no further optimization is required to obtain desired properties (e.g., binding affinities, thermal stabilities, and/or limited aggregation).

In some implementations, the antibody (or antigen-binding fragments thereof) specifically binds to a target with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) are greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) are less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶M, less than 1×10⁻⁷M, less than 1×10⁻⁸ M, less than 1×10⁻⁹ M, or less than 1×10⁻¹⁰ M. In some embodiments, the KD is less than 50 nM, 40 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M. In some embodiments, the antibody binds to a target with KD less than or equal to about 0.9 nM, 0.8 nM, 0.7 nM, 0.6 nM, 0.5 nM, 0.4 nM, 0.3 nM, 0.2 nM, or 0.1 nM.

In some embodiments, thermal stabilities are determined. The antibodies or antigen binding fragments as described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In various embodiments, substitutions are performed to a parental heavy chain antibody sequence to make a variant heavy chain antibody. In general, a heavy chain antibody variant of a parental heavy chain antibody has an antigen binding affinity that is at least 10%, at least 20%, at least 30%, at least 40%, at least 50%, at least 60%, at least 70%, at least 80%, at least 90% or at least 100% (e.g., at least 150%, at least 200%, at least 500%, at least 1000%, or up to at least 10,000%) of the binding affinity of the parental heavy chain antibody to a particular antigen. In some embodiments, a variant heavy chain antibody will comprise a single substitution as compared to a parental heavy chain antibody. However, in other embodiments, several amino acids, e.g., up to about 5 or 10 or more, are substituted as compared to the parental heavy chain antibody sequence that are derived from other human heavy chain sequences that share identity at a given position. In various embodiments, the resultant variant heavy chain antibody is tested to confirm that the desired binding affinity and/or specificity has not been significantly decreased by the replacement residues. In some embodiments, an improved variant heavy chain antibody is produced by the substitution of amino acids from a different human heavy chain sequence. In various embodiments, the VHH is at least 70%, 75%, 80%, 85%, 90%, 91%, 92%, 93%, 94%, 95%, 96%, 97%, 98%, 99%, or 100% identical to a parental VHH.

The VHH described herein can be used to make multi-specific (e.g., bispecific antibodies). In one aspect, the present disclosure provides a multi-specific antibody comprising: a first antigen binding portion and a second antigen binding portion. In some embodiments, the first antigen binding portion comprises a heavy chain variable domain (VH) and a light chain variable domain (VL), wherein the VH and VL together form an antigen-binding site that specifically binds a first epitope. In some embodiments, the first antigen binding portion comprises a VHH that specifically binds a first epitope. In some embodiments, the second antigen binding portion comprises a VHH that specifically binds a second epitope. In some embodiments, the first epitope and the second epitope are from the same antigen. In some embodiments, the first epitope and the second epitope are from different antigens.

In some embodiments, the first antigen binding portion is a full-length antibody consisting of two heavy chains and two light chains. In some embodiments, the first antigen binding portion is an antibody fragment comprising a heavy chain comprising the VH and a light chain comprising the VL. In some embodiments, the second antigen binding portion comprises a single polypeptide chain. In some embodiments, the C terminus of the second antigen binding portion is fused to the N-terminus of at least one heavy chain of the first antigen binding portion. In some embodiments, the C terminus of the second antigen binding portion is fused to the N-terminus of at least one light chain of the first antigen binding portion. In some embodiments, the N terminus of the second antigen binding portion is fused to the C-terminus of at least one heavy chain of the first antigen binding portion. In some embodiments, the N terminus of the second antigen binding portion is fused to the C-terminus of at least one light chain of the first antigen binding portion. In some embodiments, the second antigen binding portion is a Fab-like domain comprising a first polypeptide chain comprising a first VHH fused to a CH1 domain, and a second polypeptide chain comprising a second VHH fused to a CL domain.

In some embodiments, the antibody or antigen-binding fragment thereof is a tri-specific antibody. In some embodiments, the tri-specific antibody is a tri-specific VHH-Fc. In some embodiments, the tri-specific antibody comprises the same VHHs. In some embodiments, the tri-specific antibody comprises different VHHs. In some embodiments, the VHHs bind to the same epitope. In some embodiments, the VHHs bind to different epitopes.

In some embodiments, the antibody or antigen-binding fragment thereof has four or more than four VHHs. In some embodiments, in order to increase developability, at least four VHHs are combined without the addition of IgG Fc domain to construct tetra-specific VHHs. These molecules would have the added advantage of increased affinity and avidity towards the antigen compared to bi- and tri-specific VHH-Fes, despite lacking the Fc effector functions.

In some embodiments, these the antibody or antigen-binding fragment thereof (e.g., comprising 1, 2, 3, 4, 5, 6, 7, 8, 9, 10, or more than 10) has a functional Fc.

In some embodiments, the heavy-chain antibody produced by the genetically modified non-human animal described herein has a VHH domain that includes CDR1, CDR2, and CDR3. In some embodiments, the CDR3 length is between 6-23, e.g., 6, 7, 8, 9, 10, 11, 12, 13, 14, 15, 16, 17, 18, 19, 20, 21, 22 or 23. In some embodiments, the CDR3 length is at least 6, at least 7, at least 8, at least 9, at least 10, at least 11, at least 12, at least 13, at least 14, at least 15, at least 16, at least 17, at least 18, at least 19, at least 20, at least 21, at least 22, or at least 23.

### Methods of Making Genetically Modified Animals

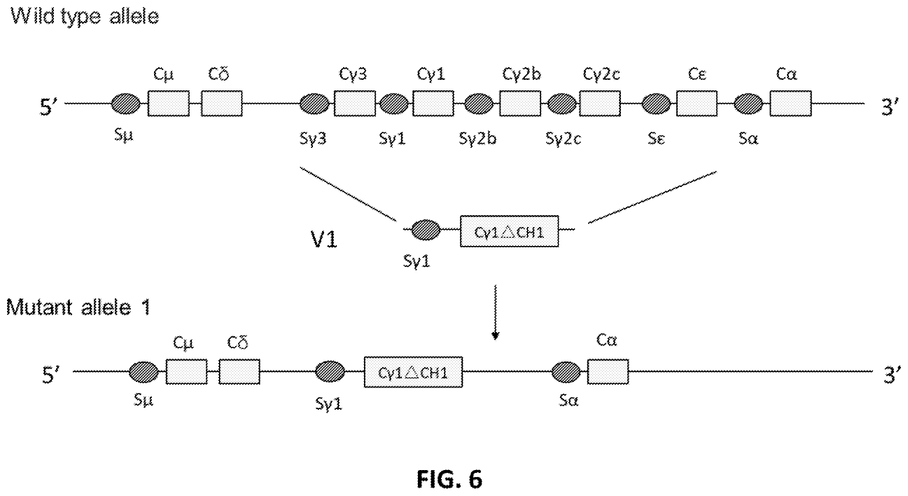

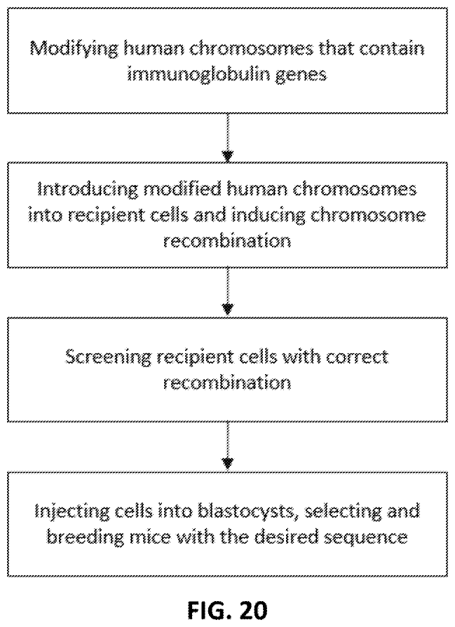

The genetically modified animals can be made by modifying the immunoglobulin gene locus. FIGS. 6-12 shows workflows of genetic modifications using targeting vectors V1-V7, respectively. FIG. 20 shows the methods of making the humanized animals. In some embodiments, the methods first involve modifying the human immunoglobulin locus on the human chromosome. The modified human chromosomes are then introduced into the mouse recipient cell. The human immunoglobulin variable region is then introduced into the corresponding region of the mouse genome by direct replacement. Then, the recipient cells are screened. In some embodiments, the cells do not contain the human chromosomes. The cells are then injected to blastocysts to prepare chimeric mice. Subsequent breeding can be performed to obtain mice containing intact humanized immunoglobulin locus.

Several other techniques may be used in making genetically modified animals, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

The genetic modification process can involve replacing endogenous sequence with human sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous sequence with human sequence.

In some embodiments, the methods for deleting CH1 sequence in an IGHG gene involve one or the combination of the following methods. These modifications can be performed in various cells. In some embodiments, the cell is a stem cell, an embryonic stem cell, or a fertilized egg cell.

In some embodiments, a sequence starting from the Sγ3 switch region to Cε is knocked out, then a sequence comprising the Sγ1 switch region and a Cγ1 sequence without CH1 (abbreviated Cγ1ΔCH1) is inserted. As a result, the Cγ1ΔCH1 is operably linked with Sγ1.

In some embodiments, a sequence comprising the Sγ1 switch region and a Cγ1 sequence without CH1 (abbreviated Cγ1ΔCH1) is used to directly replace all sequences starting from Sγ3 switch region to Cε (FIG. 6). As a result, the Cγ1ΔCH1 is operably linked with Sγ1.

In some embodiments, Sγ3 switch region and the Cγ3 sequence are knocked out first, and then a Cγ1 sequence without CH1 (abbreviated as Cγ1ΔCH1) is used to replace all the sequences from Cγ1 to Cα. As a result, the Cγ1ΔCH1 is operably linked with Sγ1.

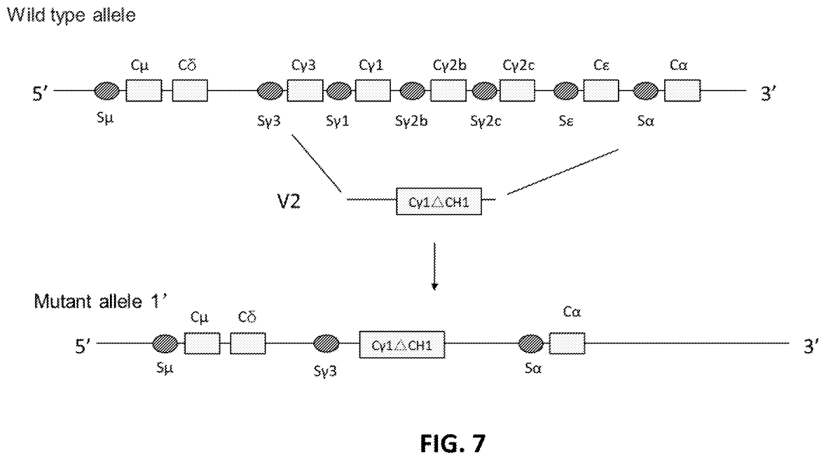

In some embodiments, a Cγ1 sequence without CH1 (abbreviated as Cγ1ΔCH1) is used to directly replace all sequences starting from Cγ3 to Cε (FIG. 7). As a result, the Cγ1ΔCH1 is operably linked with Sγ3.

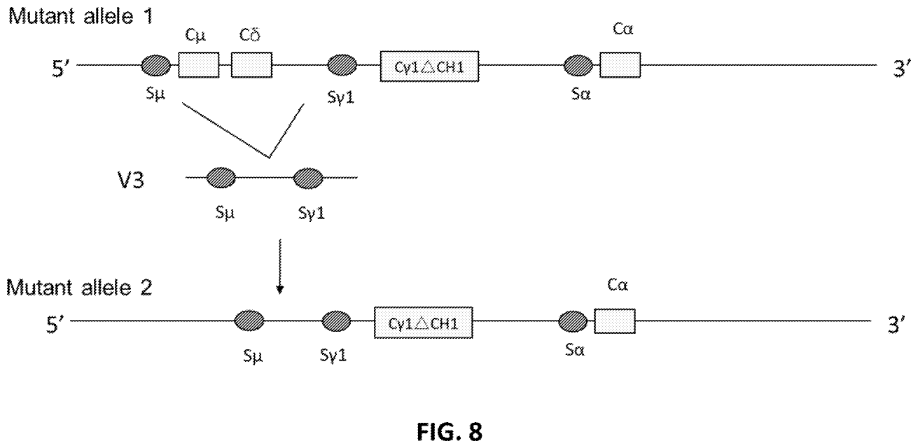

In some embodiments, based on the allele shown in FIG. 6, a sequence including Cμ and Co is knocked out (FIG. 8). As a result, both IGHM and IGHδ genes are knocked out, and the Cγ1ΔCH1 is operably linked with Sγ1.

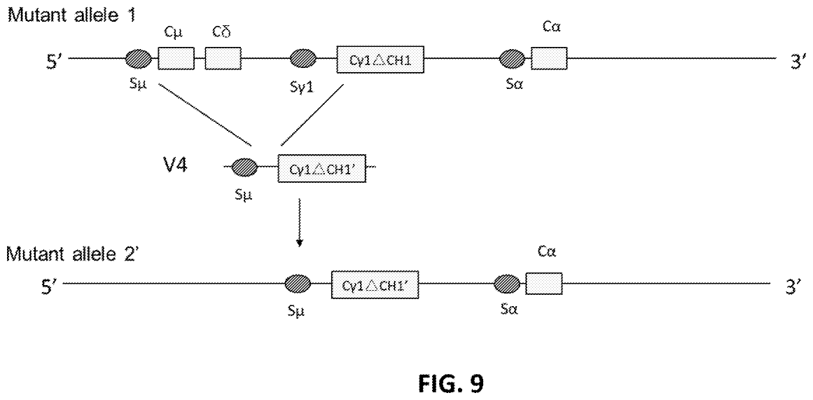

In some embodiments, based on the allele shown in FIG. 6, a sequence including Cμ, Cδ, and Sγ1 is knocked out (FIG. 9). As a result, both IGHM and IGHδ genes are knocked out, and the Cγ1ΔCH1′ is operably linked with Sμ.

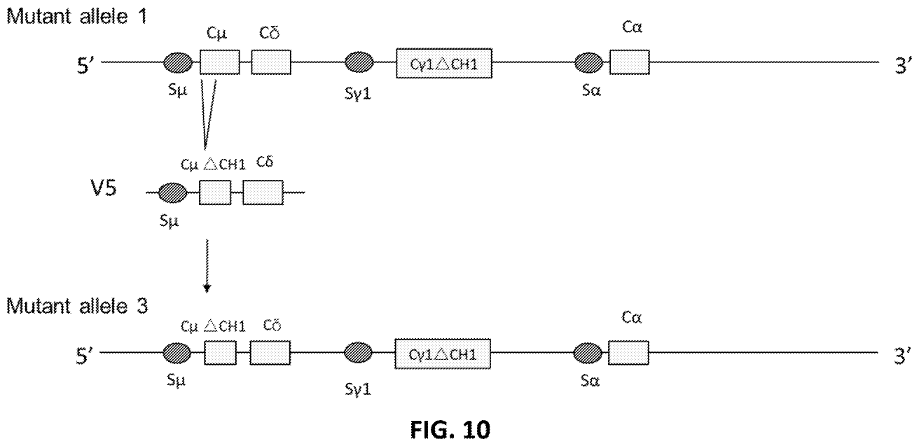

In some embodiments, based on the allele shown in FIG. 6, the CH1 coding region of Cμ is knocked out (FIG. 10). As a result, the modified locus includes sequences encoding IgM lacking a CH1 domain, IgD, and IgG1 lacking a CH1 domain. In addition, the Cγ1ΔCH1 is operably linked with Sγ1.

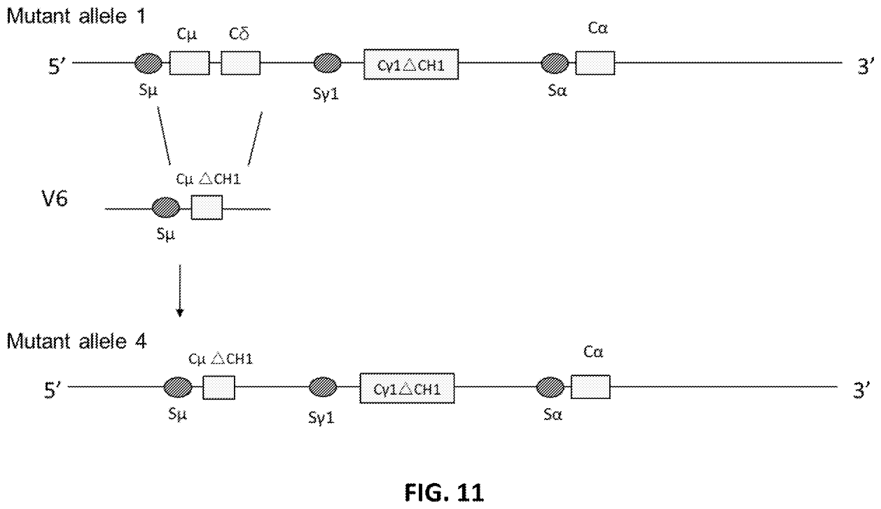

In some embodiments, based on the allele shown in FIG. 6, a Cμ sequence without the CH1 coding region (abbreviated as CμΔCH1) is used to directed replace a sequence including Cμ and Cδ (FIG. 11). As a result, the modified locus includes sequences encoding IgM lacking a CH1 domain, and IgG1 lacking a CH1 domain. In addition, the Cγ1ΔCH1 is operably linked with Sγ1.

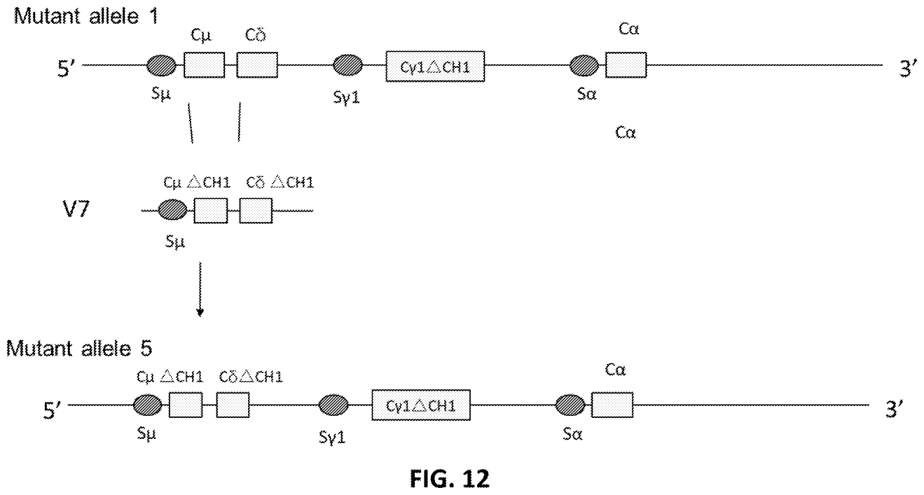

In some embodiments, based on the allele shown in FIG. 6, a sequence including CμΔCH1 and Co without the CH1 coding region (abbreviated as CδΔCH1) is used to directed replace a sequence including Cμ and Cδ (FIG. 12). As a result, the modified locus includes sequences encoding IgM lacking a CH1 domain, IgD lacking a CH1 domain, and IgG1 lacking a CH1 domain. In addition, the Cγ1ΔCH1 is operably linked with Sγ1.

In some embodiments, provided herein is a genetically modified non-human animal comprising a modified immunoglobulin heavy chain constant region locus. In some embodiments, the modified immunoglobulin heavy chain constant region locus comprises a sequence that is at least 80%, 85%, 90%, 95%, 96%, 97%, 98%, 99%, or 100 identical to the sequence of Cγ1ΔCH1, Cγ1ΔCH1′, CμΔCH1, or CδΔCH1. In some embodiments, the sequence of Cγ1ΔCH1′ includes a deletion of at least 1, at least 2, at least 3, or at least 4 nucleotides at the 5′ end of the sequence of Cγ1ΔCH1 (SEQ ID NO: 1).

The present disclosure also relates to a genetically modified non-human animal comprising a nucleic acid sequence, wherein the nucleic acid sequence can be selected from the group consisting of:
a) a nucleic acid sequence as shown in SEQ ID NO: 1, 8, 9, 10, 13, or 41;
b) a nucleic acid sequence that is able to hybridize to the nucleotide sequence as shown in SEQ ID NO: 1, 8, 9, 10, 13, or 41 under a low stringency condition or a strict stringency condition;
c) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 1, 8, 9, 10, 13, or 41; and
d) a nucleic acid sequence that encodes an amino acid sequence, wherein the amino acid sequence has a homology of at least 90% with or at least 90% identical to the amino acid sequence of endogenous IgG, IgM, IgD, or IgA.

The method further involves transplanting the genetically modified cell to the fallopian tube or uterus of the recipient female non-human mammal, allowing the cell to develop in the uterus of the female non-human mammal. In some embodiments, experiments are performed to identify the germline transmission in the offspring the genetically modified genes.

In some embodiments, the methods for making a genetically modified, humanized animal, can also include the step of replacing at an endogenous locus (or site), a nucleic acid (e.g., V, D, J regions, or V, J regions) with a corresponding region of human sequence. The sequence can include a region (e.g., a part or the entire region) of IGHV, IGHD, IGHJ, IGKV, and/or IGKJ genes. In some embodiments, the replacement is mediated by homologous recombination. In some embodiments, the replacement is mediated by Cre recombinase.

The 5′ end homology arm and/or the 3′ end homology arm can have a desired length to facilitate homologous recombination. In some embodiments, the homology arm is about or at least 1, 2, 3, 4, 5, 6, 7, 8, 9, 10, 20, 30, 40, or 50 kb (e.g., about 3 kb). In some embodiments, the homology arm is less than 1, 2, 3, 4, 5, 6, 7, 8, 9, 10, 20, 30, 40, or 50 kb.

In some embodiments, the vector may also optionally include a reporter protein, e.g., a luciferase (e.g., Gluc) or a fluorescent protein (e.g., EGFP, BFP, etc.).

These modifications can be performed in various cells. In some embodiments, the cell is a stem cell, an embryonic stem cell, or a fertilized egg cell.

The present disclosure further provides a method for establishing a humanized animal model, involving the following steps:
(a) providing the cell (e.g. a fertilized egg cell) based on the methods described herein;
(b) culturing the cell in a liquid culture medium;
(c) transplanting the cultured cell to the fallopian tube or uterus of the recipient female non-human mammal, allowing the cell to develop in the uterus of the female non-human mammal;
(d) identifying the germline transmission in the offspring genetically modified humanized non-human mammal of the pregnant female in step (c).

In some embodiments, the non-human mammal in the foregoing method is a mouse (e.g., a C57 mouse, a BALB/c mouse, or a C57BL/6 mouse).

In some embodiments, the non-human mammal in step (c) is a female with pseudo pregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the methods described above.

Cells, tissues, and animals (e.g., mouse) are also provided that comprise the nucleotide sequences as described herein, as well as cells, tissues, and animals (e.g., mouse) that express humanized or chimeric antibodies from an endogenous non-human locus.

The present disclosure also provides various targeting vectors (e.g., vectors that are useful for making the genetically modified animals). In some embodiments, the vector can comprise: a) a DNA fragment homologous to the 5′ end of a region to be altered (5′ homology arm); b) a sequence comprising desired genetic elements (e.g., LoxP recognition site, drug resistance genes, and/or reporter genes etc.); and c) a second DNA fragment homologous to the 3′ end of the region to be altered (3′ homology arm). The disclosure also relates to a cell comprising the targeting vectors as described herein.

In some embodiments, the genes in the cell are heterozygous. In some embodiments, the genes in the cell are homozygous.

In some embodiments, the non-human mammalian cell is a mouse cell. In some embodiments, the cell is a fertilized egg cell.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, or 400 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences. For purposes of illustration, the comparison of sequences and determination of percent identity between two sequences can be accomplished using a Blossum 62 scoring matrix with a gap penalty of 12, a gap extend penalty of 4, and a frameshift gap penalty of 5 when appropriate.

The percentage of residues conserved with similar physicochemical properties (percent homology), e.g. leucine and isoleucine, can also be used to measure sequence similarity. Families of amino acid residues having similar physicochemical properties have been defined in the art. These families include e.g., amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage. Thus, the present disclosure also provides an amino acid sequence that has at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% homology percentage to any amino acid sequence as described herein, or a nucleic acid encoding these amino acid sequences.

### Methods of Using Genetic Modified Animals

The genetic modified animals can be used to generate heavy-chain antibodies that can bind specifically to a target. In some embodiments, the target (e.g., a protein or a fragment of the protein) can be used as an immunogen to generate antibodies in these animals using standard techniques for polyclonal and monoclonal antibody preparation. In some embodiments, the genetic modified animal is exposed to a selected antigen for a time and under conditions which permit the animal to produce antibody specific for the antigen.

Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence and encompasses an epitope of the protein such that an antibody raised against the peptide forms a specific immune complex with the protein.

An immunogen typically is used to prepare antibodies by immunizing a suitable subject (e.g., the genetically modified animal as described herein). An appropriate immunogenic preparation can contain, for example, a recombinantly-expressed or a chemically-synthesized polypeptide (e.g., a fragment of the protein). The preparation can further include an adjuvant, such as Freund's complete or incomplete adjuvant, or a similar immunostimulatory agent.

The antibody titer in the immunized subject can be monitored over time by standard techniques, such as with an enzyme-linked immunosorbent assay (ELISA) using the immobilized polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

In one aspect, the disclosure provides a mouse that comprises a modification of an endogenous immunoglobulin heavy chain locus, wherein the mouse produces a B cell that comprises a rearranged immunoglobulin sequence operably linked to a heavy chain constant region gene sequence. In some embodiment, the rearranged immunoglobulin sequence operably linked to the heavy chain constant region gene sequence comprises a human heavy chain V, D. and/or J sequence. In some embodiments, the heavy chain constant region gene sequence comprises a human or a mouse heavy chain sequence selected from the group consisting of a CH1, a hinge, a CH2, a CH3, and a combination thereof.

In one aspect, the disclosure relates to a method for making a somatically mutated heavy chain antibody in an animal. The method involves immunizing an animal with an antigen, maintaining the animal under conditions sufficient to initiate an immune response to the antigen; and, isolating from the animal a somatically mutated heavy chain antibody comprising a variable domain that is derived from the human or endogenous heavy chain immunoglobulin variable region gene segment, and wherein the somatically mutated heavy chain antibody specifically binds the antigen. In some embodiments, the animal comprises an unrearranged human or endogenous heavy chain immunoglobulin variable region gene segment, and wherein the animal lacks a nucleotide sequence of at least one allele that encodes a functional IgG CH1 domain, and wherein the animal expresses an IgM that comprises a CH1 domain;

The B cells or spleen cells can comprise a rearranged non-mouse immunoglobulin variable gene sequence, e.g., operably linked to a mouse immunoglobulin constant region gene. The sequences for encoding human heavy chain variable region are determined. The sequences can be determined by e.g., sequencing the hybridoma of interest or B cells. In some embodiments, single B cell screening is used. It can screen the natural antibody repertoire without the need for hybridoma fusion and combinatorial display. For example, B cells can be mixed with a panel of DNA-barcoded antigens, such that both the antigen barcode(s) and B-cell receptor (BCR) sequences of individual B cells are recovered via single-cell sequencing protocols.

The antibodies can be further modified to obtain a humanized antibody or a human antibody, e.g., by operably linking the sequence encoding human heavy chain variable region to a sequence encoding a human heavy chain constant region.

In some embodiments, if the mouse expresses a protein that is very similar to the antigen of interest, it can be difficult to elicit an immune response in the mouse. This is because during immune cell development, B-cells and T-cells that recognize MHC molecules bound to peptides of self-origin are deleted from the repertoire of immune cells. In those cases, the humanized mouse can be further modified. The corresponding gene in the mouse can be knocked out, and the mouse is then exposed to the antigen of interest. Because the mouse does not go through negative selection for the gene product, the mouse can generate an antibody that can specifically bind to the target easily.

The disclosure also provides methods of making antibodies, nucleic acids, cells, tissues (e.g., spleen tissue). In some embodiments, the methods involve exposing the animal as described herein to the antigen. Antibodies (e.g., hybrid antibodies), nucleic acids encoding the antibodies, cells, and/or tissues (e.g., spleen tissue) can be obtained from the animal. In some embodiments, the nucleic acids encoding the variable regions are determined, e.g., by sequencing. In some embodiments, the nucleic acid encoding the human heavy chain immunoglobulin variable region can be operably linked with a nucleic acid encoding a human heavy chain immunoglobulin constant region. In some embodiments, the cells containing the nucleic acids as described herein are cultured and the antibodies are collected.

In some embodiments, no mouse immunoglobulin V, D. J genes (e.g., no mouse IGHV, IGHD, IGHJ, IGKV, or IGKJ genes) contributes to the heavy chain variable region sequence. In some embodiments, the heavy chain variable region sequence produced by the animal are fully human, and are completely contributed by human immunoglobulin V, D, J genes (e.g., human IGHV, IGHD, IGHJ, IGKV, and IGKJ genes). In some embodiments, the rearranged VDJ sequences can further undergo somatic hypermutations.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

Additional modifications to the antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. (Cancer Res. 53:2560-2565, 1993). Alternatively, an antibody can be engineered which has dual Fc regions (see, for example, Stevenson et al., Anti-Cancer Drug Design 3:219-230, 1989).

In some embodiments, a covalent modification can be made to the antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

### Transferrin Receptor 1 (TFR1)

TFR1, also known as cluster of differentiation 71 (CD71), is widely expressed and can bind to transferrin (Tf) with high affinity. Human TFR1 is a 90 kDa type II transmembrane glycoprotein consisting of 760 amino acids that is found as a dimer (180 kDa) linked by disulfide bonds on the cell surface. The TFR1 monomer is composed of a large extracellular, C-terminal domain of 671 amino acids containing the Tf-binding site, a transmembrane domain (28 amino acids), and an intracellular N-terminal domain (61 amino acids). The C-terminal extracellular domain contains three N-linked glycosylation sites at asparagine residues 251, 317, and 727 and one O-linked glycosylation site at threonine 104, which are all required for adequate function of the receptor.

Transferrin (Tf) It is an 80 kDa glycoprotein composed of two 40 kDa subunits, known as the N- and C-lobes that are separated by a short linker sequence. Each subunit is capable of binding to one free ferric iron (Fe³⁺) and thus, Tf may have up to two atoms of iron attached. Tf in its iron free form, apo-Tf, binds Fe³⁺ with high efficiency in the blood and transports it to the cell surface for internalization through the interaction with TFR1. As a membrane protein regulating iron import, TFR1 is a member of the TFR family that shows nanomolar affinity to transferrin (Tf) bound to Fe³⁺. The complex of Tf-TFR1 is internalized through endocytosis mediated by clathrin, and Fes is disassociated from Tf when pH decreases to 5.5. At this pH, apo-Tf and TFR1 are still associated and recycled to cell surface with physiological pH, so the former is released.

Iron uptake by transferrin receptor is an important way for cancer cells to absorb iron, thus accumulating evidence has proven that TFR1 participated in tumor onset and progression, and its expression was dysregulated significantly in many cancers. The relationship between TFR1 and cancers has been revealed, rendering TFR1 a valuable pharmaceutical target for intervening with cancers.

TFR1 expressed on the endothelial cells of the blood-brain barrier is used also in preclinical research to allow the delivery of large molecules including antibodies into the brain. The TFR1 targeting antibodies can cross the blood-brain barrier, without interfering with the uptake of iron.

A detailed description of TFR1, Tf, and their functions can be found, e.g., in Candelaria, P. V., et al. “Antibodies targeting the transferrin receptor 1 (TfR1) as direct anti-cancer agents.” Frontiers in Immunology 12 (2021): 607692; and Shen, Y., et al. “Transferrin receptor 1 in cancer: a new sight for cancer therapy.” American Journal of Cancer Research 8.6 (2018): 916; each of which is incorporated by reference in its entirety.

### Heavy Chain Single Variable Domain (VHH) Antibodies

Monoclonal and recombinant antibodies are important tools in medicine and biotechnology. Like all mammals, camelids (e.g., llamas) can produce conventional antibodies made of two heavy chains and two light chains bound together with disulfide bonds in a Y shape (e.g., IgG1). However, they also produce two unique subclasses of IgG: IgG2 and IgG3, also known as heavy chain IgG. These antibodies are made of only two heavy chains, which lack the CH1 region but still bear an antigen-binding domain at their N-terminus called VHH (or nanobody). Conventional Ig require the association of variable regions from both heavy and light chains to allow a high diversity of antigen-antibody interactions. Although isolated heavy and light chains still show this capacity, they exhibit very low affinity when compared to paired heavy and light chains. The unique feature of heavy chain IgG is the capacity of their monomeric antigen binding regions to bind antigens with specificity, affinity and especially diversity that are comparable to conventional antibodies without the need of pairing with another region. This feature is mainly due to a couple of major variations within the amino acid sequence of the variable region of the two heavy chains, which induce deep conformational changes when compared to conventional Ig. Major substitutions in the variable regions prevent the light chains from binding to the heavy chains, but also prevent unbound heavy chains from being recycled by the immunoglobulin binding proteins.

The single variable domain of these antibodies (designated VHH, sdAb, or nanobody) is the smallest antigen-binding domain generated by adaptive immune systems. The third Complementarity Determining Region (CDR3) of the variable region of these antibodies has been found to be twice as long as the conventional ones. This results in an increased interaction surface with the antigen as well as an increased diversity of antigen-antibody interactions, which compensates the absence of the light chains. With a long complementarity-determining region 3 (CDR3). VHHs can extend into crevices on proteins that are not accessible to conventional antibodies, including functionally interesting sites such as the active site of an enzyme or the receptor-binding canyon on a virus surface.

VHHs offer numerous other advantages compared to conventional antibodies carrying variable domains (VH and VL) of conventional antibodies, including higher stability, solubility, expression yields, and refolding capacity, as well as better in vivo tissue penetration and internalization. Moreover, in contrast to the VH domains of conventional antibodies, VHH do not display an intrinsic tendency to bind to light chains. Since VHH do not bind to VL domains, it is much easier to reformat VHHs into multi-specific (e.g., bispecific antibody) constructs than constructs containing conventional VH-VL pairs or single domains based on VH domains.

The disclosure provides e.g., anti-TFR1 antibodies, the modified antibodies thereof, the chimeric antibodies thereof, and the humanized antibodies thereof.

The CDR sequences for 23B8, and 23B8 derived antibodies (e.g., humanized antibodies) include CDRs of the VHH domain as set forth in SEQ ID NOs: 42, 43, and 44, respectively, as defined by Kabat numbering. The CDRs can also be defined by IMGT system. Under the IMGT numbering, the CDRs of the VHH domain are set forth in SEQ ID NOs: 54, 55, and 56, respectively.

The CDR sequences for 24A1, and 24A1 derived antibodies (e.g., humanized antibodies) include CDRs of the VHH domain as set forth in SEQ ID NOs: 45, 46, and 47, respectively, as defined by Kabat numbering. The CDRs can also be defined by IMGT system. Under the IMGT numbering, the CDRs of the VHH domain are set forth in SEQ ID NOs: 57, 58, and 59, respectively.

The CDR sequences for 24C9, and 24C9 derived antibodies (e.g., humanized antibodies) include CDRs of the VHH domain as set forth in SEQ ID NOs: 48, 49, and 50, respectively, as defined by Kabat numbering. The CDRs can also be defined by IMGT system. Under the IMGT numbering, the CDRs of the VHH domain are set forth in SEQ ID NOs: 60, 61, and 62, respectively.

The CDR sequences for 24G5, and 24G5 derived antibodies (e.g., humanized antibodies) include CDRs of the VHH domain as set forth in SEQ ID NOs: 51, 52, and 53, respectively, as defined by Kabat numbering. The CDRs can also be defined by IMGT system. Under the IMGT numbering, the CDRs of the VHH domain are set forth in SEQ ID NOs: 63, 64, and 65, respectively.

The amino acid sequence for the VHH domain of 23B8 antibody is set forth in SEQ ID NO: 66. The amino acid sequence for the VHH domain of 24A1 antibody is set forth in SEQ ID NO: 67. The amino acid sequence for the VHH domain of 24C9 antibody is set forth in SEQ ID NO: 68. The amino acid sequence for the VHH domain of 24G5 antibody is set forth in SEQ ID NO: 69.

The amino acid sequences for various modified or humanized VHH are also provided. As there are different ways to modify or humanize a heavy-chain antibody (e.g., a sequence can be modified with different amino acid substitutions), the VHH domain of a heavy-chain antibody can have more than one version of humanized sequences. In some embodiments, the humanized VHH domain is at least 80%, 85%, 90%, 91%, 92%, 93%, 94%, 95%, 96%, 97%, 98%, or 99% identical to any sequence of SEQ ID NOS: 66-69.

Furthermore, in some embodiments, the antibodies or antigen-binding fragments thereof described herein can also contain one, two, or three VHH domain CDRs selected from the group of SEQ ID NOs: 42-44, SEQ ID NOs: 45-47, SEQ ID NOs: 48-50, SEQ ID NOs: 51-53, SEQ ID NOs: 54-56, SEQ ID NOs: 57-59, SEQ ID NOs: 60-62, and SEQ ID NOs: 63-65.

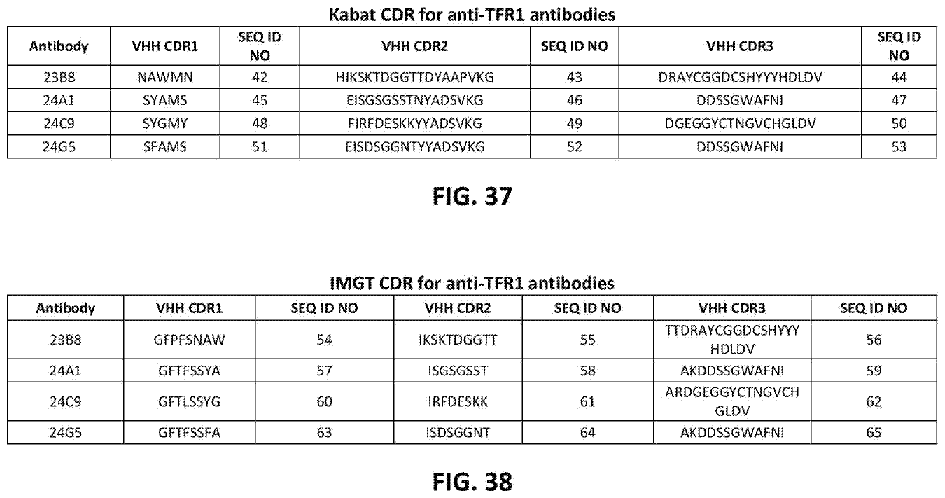

In some embodiments, the antibodies can have a heavy chain single variable domain (VHH) comprising complementarity determining regions (CDRs) 1, 2, 3, wherein the CDR1 region comprises or consists of an amino acid sequence that is at least 80%, 85%, 90%, or 95% identical to a selected VHH CDR1 amino acid sequence, the CDR2 region comprises or consists of an amino acid sequence that is at least 80%, 85%, 90%, or 95% identical to a selected VHH CDR2 amino acid sequence, and the CDR3 region comprises or consists of an amino acid sequence that is at least 80%, 85%, 90%, or 95% identical to a selected VHH CDR3 amino acid sequence. The selected VHH CDRs 1, 2, 3 amino acid sequences is shown in FIG. 37 and FIG. 38.

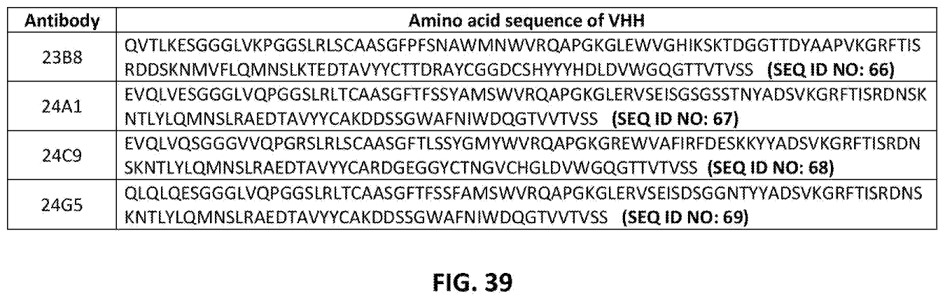

In some embodiments, the antibody or an antigen-binding fragment described herein can contain a heavy chain single variable domain (VHH) containing one, two, or three of VHH CDR1 with zero, one or two amino acid insertions, deletions, or substitutions; VHH CDR2 with zero, one or two amino acid insertions, deletions, or substitutions; VHH CDR3 with zero, one or two amino acid insertions, deletions, or substitutions, wherein VHH CDR1, VHH CDR2, and VHH CDR3 are selected from the CDRs in FIG. 39.

In some embodiments, the antibody or an antigen-binding fragment described herein can contain a heavy chain single variable domain (VHH) containing one, two, or three of the CDRs of SEQ ID NO: 42 with zero, one or two amino acid insertions, deletions, or substitutions; SEQ ID NO: 43 with zero, one or two amino acid insertions, deletions, or substitutions; SEQ ID NO: 44 with zero, one or two amino acid insertions, deletions, or substitutions.

In some embodiments, the antibody or an antigen-binding fragment described herein can contain a heavy chain single variable domain (VHH) containing one, two, or three of the CDRs of SEQ ID NO: 45 with zero, one or two amino acid insertions, deletions, or substitutions; SEQ ID NO: 46 with zero, one or two amino acid insertions, deletions, or substitutions; SEQ ID NO: 47 with zero, one or two amino acid insertions, deletions, or substitutions.

In some embodiments, the antibody or an antigen-binding fragment described herein can contain a heavy chain single variable domain (VHH) containing one, two, or three of the CDRs of SEQ ID NO: 48 with zero, one or two amino acid insertions, deletions, or substitutions; SEQ ID NO: 49 with zero, one or two amino acid insertions, deletions, or substitutions; SEQ ID NO: 50 with zero, one or two amino acid insertions, deletions, or substitutions.

In some embodiments, the antibody or an antigen-binding fragment described herein can contain a heavy chain single variable domain (VHH) containing one, two, or three of the CDRs of SEQ ID NO: 51 with zero, one or two amino acid insertions, deletions, or substitutions; SEQ ID NO: 52 with zero, one or two amino acid insertions, deletions, or substitutions; SEQ ID NO: 53 with zero, one or two amino acid insertions, deletions, or substitutions.

In some embodiments, the antibody or an antigen-binding fragment described herein can contain a heavy chain single variable domain (VHH) containing one, two, or three of the CDRs of SEQ ID NO: 54 with zero, one or two amino acid insertions, deletions, or substitutions; SEQ ID NO: 55 with zero, one or two amino acid insertions, deletions, or substitutions; SEQ ID NO: 56 with zero, one or two amino acid insertions, deletions, or substitutions.

In some embodiments, the antibody or an antigen-binding fragment described herein can contain a heavy chain single variable domain (VHH) containing one, two, or three of the CDRs of SEQ ID NO: 57 with zero, one or two amino acid insertions, deletions, or substitutions; SEQ ID NO: 58 with zero, one or two amino acid insertions, deletions, or substitutions; SEQ ID NO: 59 with zero, one or two amino acid insertions, deletions, or substitutions.

In some embodiments, the antibody or an antigen-binding fragment described herein can contain a heavy chain single variable domain (VHH) containing one, two, or three of the CDRs of SEQ ID NO: 60 with zero, one or two amino acid insertions, deletions, or substitutions; SEQ ID NO: 61 with zero, one or two amino acid insertions, deletions, or substitutions; SEQ ID NO: 62 with zero, one or two amino acid insertions, deletions, or substitutions.

In some embodiments, the antibody or an antigen-binding fragment described herein can contain a heavy chain single variable domain (VHH) containing one, two, or three of the CDRs of SEQ ID NO: 63 with zero, one or two amino acid insertions, deletions, or substitutions; SEQ ID NO: 64 with zero, one or two amino acid insertions, deletions, or substitutions; SEQ ID NO: 65 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on a combination numbering scheme. In some embodiments, the CDR is determined based on IMGT numbering scheme.

The disclosure also provides antibodies or antigen-binding fragments thereof that bind to TFR1 (human TFR1). The antibodies or antigen-binding fragments thereof contain a heavy chain single variable region (VHH) comprising or consisting of an amino acid sequence that is at least 80%, 85%, 90%, or 95% identical to a selected VHH sequence. In some embodiments, the selected VHH sequence is SEQ ID NO: 66. In some embodiments, the selected VHH sequence is SEQ ID NO: 67. In some embodiments, the selected VHH sequence is SEQ ID NO: 68. In some embodiments, the selected VHH sequence is SEQ ID NO: 69.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of illustration, the comparison of sequences and determination of percent identity between two sequences can be accomplished, e.g., using a Blossum 62 scoring matrix with a gap penalty of 12, a gap extend penalty of 4, and a frameshift gap penalty of 5.

The disclosure also provides nucleic acid comprising a polynucleotide encoding a polypeptide comprising an immunoglobulin heavy chain single variable domain (VHH). The VHH comprises CDRs as shown in FIG. 37 and FIG. 38, or has sequences as shown in FIG. 39.

The antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof.

In some embodiments, the antibodies or antigen-binding fragments thereof comprises an Fc domain that can be originated from various types (e.g., IgG, IgE, IgM, IgD, IgA, and IgY), class (e.g., IgG1, IgG2, IgG3, IgG4, IgA1, and IgA2), or subclass. In some embodiments, the Fc domain is originated from an IgG antibody or antigen-binding fragment thereof. In some embodiments, the Fc domain comprises one, two, three, four, or more heavy chain constant regions.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp 120 exterior envelope glycoprotein.” Journal of virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

In some embodiments, the antibody or antigen-binding fragment thereof comprises a heavy chain single variable domain (VHH) CDR1 selected from SEQ ID NOs: 42, 45, 48, 51, 54, 57, 60, and 63

In some embodiments, the antibody or antigen-binding fragment thereof comprises a heavy chain single variable domain (VHH) CDR2 selected from SEQ ID NOs: 43, 46, 49, 52, 55, 58, 61, and 64.

In some embodiments, the antibody or antigen-binding fragment thereof comprises a heavy chain single variable domain (VHH) CDR3 selected from SEQ ID NOs: 44, 47, 50, 53, 56, 59, 62, and 65.

### Antibody Characteristics

TFR1 performs a critical role in cellular iron uptake through the interaction with iron-bound TF. Iron is required for multiple cellular processes and is essential for DNA synthesis and, thus, cellular proliferation. Due to its central role in cancer cell pathology, malignant cells often overexpress TFR1 and this increased expression can be associated with poor prognosis in different types of cancer. The elevated levels of TfR1 expression on malignant cells, together with its extracellular accessibility, ability to internalize, and central role in cancer cell pathology make this receptor an attractive target for antibody-mediated therapy.

In some embodiments, the antibodies or antigen-binding fragments thereof described herein cannot block the binding between TFR1 and TF. In some embodiments, the antibodies or antigen-binding fragments thereof described herein can block the binding between TFR1 and TF. In some embodiments, the antibodies or antigen-binding fragments thereof described herein can be conjugated to anti-cancer agents that are internalized by receptor-mediated endocytosis. In some embodiments, the antibodies or antigen-binding fragments thereof described herein can disrupt the function of the receptor. In some embodiments, the antibodies or antigen-binding fragments thereof described herein cannot induce Fc effector functions, therefore preventing or ameliorating their negative effects to normal cells.

The disclosure provides antibodies or antigen-binding fragments thereof comprising a human Fc domain, which induce Fc-dependent effector functions by at least or about at least or about 1 fold, at least or about 2 folds, at least or about 3 folds, at least or about 4 folds, at least or about 5 folds, at least or about 6 folds, at least or about 7 folds, at least or about 8 folds, at least or about 9 folds, at least or about 10 folds, at least or about 20 folds, at least or about 30 folds, at least or about 40 folds, at least or about 50 folds, or at least or about 100 folds as compared when no antibodies or antigen-binding fragments thereof as described herein are present.

The disclosure provides antibodies or antigen-binding fragments thereof comprising a human Fc domain, which induce host immune response by at least or about at least or about 1 fold, at least or about 2 folds, at least or about 3 folds, at least or about 4 folds, at least or about 5 folds, at least or about 6 folds, at least or about 7 folds, at least or about 8 folds, at least or about 9 folds, at least or about 10 folds, at least or about 20 folds, at least or about 30 folds, at least or about 40 folds, at least or about 50 folds, or at least or about 100 folds as compared when no antibodies or antigen-binding fragments thereof as described herein are present.

The disclosure provides antibodies or antigen-binding fragments thereof that can internalize into human brain cells (e.g., cortical microvascular endothelial cells) that the endocytosis rate is at least 50%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%. In some embodiments, the endocytosis rate of the antibodies or antigen-binding fragments thereof described herein is at least 1-fold, 2-fold, 3-fold, 4-fold, 5-fold, 6-fold, 7-fold, 8-fold, 9-fold, 10-fold, 50-fold, 100-fold, 500-fold, or 1000-fold as compared to that of an isotype control antibody.

In some embodiments, provided herein is an antibody or antigen-binding fragment thereof comprising a single heavy chain. In some embodiments, provided herein is an antibody or antigen-binding fragment thereof comprising a pair of heavy chains. In some embodiments, the heavy chain pair is linked by disulfide bonds. In some embodiments, the heavy chain pair comprises knob-in-hole modifications. In some embodiments, the heavy chain comprises a human IgG Fc domain. In some embodiments, the antibody or antigen-binding fragment thereof comprises in each heavy chain at least 1, 2, 3, 4, 5, 6, 7, 8, 9, or 10 VHH domains. In some embodiments, the VHH domains in each heavy chain specifically bind to the same epitope. In some embodiments, the VHH domains in each heavy chain specifically bind to different epitopes. In some embodiments, the VHH domains in each heavy chain bind to at least 1, 2, 3, 4, or 5 different epitopes.

In some embodiments, the antibody or antigen-binding fragment thereof is a bi-specific antibody, or tri-specific antibody. In some embodiments, the antibody or antigen-binding fragment thereof can specifically bind to at least 4, 5, or 6 antigens.

In some embodiments, the antibody (or antigen-binding fragment thereof) specifically binds to TFR1 with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶ M, less than 1×10⁻⁷M, less than 1×10⁻⁸ M, less than 1×10⁻⁹ M, or less than 1×10⁻¹⁰ M. In some embodiments, the KD is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M. General techniques for measuring the affinity of an antibody for an antigen include, e.g., ELISA, RIA, and surface plasmon resonance (SPR). In some embodiments, the antibody binds to human TFR1, monkey TFR1, mouse TFR1, or chimeric TFR1. In some embodiments, the antibody does not bind to human TFR1, monkey TFR1, mouse TFR1, or chimeric TFR1. In some embodiments, thermal stabilities are determined. The antibodies or antigen-binding fragments as described herein can have a Tm (melting temperature) greater than 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm is less than 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. The antibodies or antigen-binding fragments as described herein can have a Tagg (aggregation temperature, e.g., Tagg at 266 nm (Tagg266) or Tagg at 473 nm (Tagg473)) great than 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tagg is less than 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the Fc region is human IgG1, human IgG2, human IgG3, or human IgG4.

In some embodiments, the antibodies or antigen binding fragments thereof have a functional Fc region. In some embodiments, the antibodies or antigen binding fragments thereof comprise a human IgG1 Fc region. In some embodiments, the human IgG1 Fc region comprises an amino acid sequence that is at least 80%, 85%, 90%, 95%, 96%, 97%, 98%, 99%, or 100% identical to SEQ ID NO: 75.

In some embodiments, the antibodies or antigen binding fragments do not have an Fc region. For example, the antibody (or antigen-binding fragment thereof) is a polypeptide comprising one or more VHH domains that are interconnected by linker peptides. In some embodiments, the antibody comprises at least 1, 2, 3, 4, 5, 6, 7, 8, 9, or 10 VHH domain. In some embodiments, the VHH domains specifically bind to the same epitope. In some embodiments, the VHH domains bind to different epitopes. In some embodiments, the VHH domains bind to at least 1, 2, 3, 4, or 5 different epitopes.

In some embodiments, the antibodies or antigen binding fragments thereof do not have a functional Fc region. In some embodiments, the Fc region has LALA mutations (L234A and L235A mutations in EU numbering), or LALA-PG mutations (L234A, L235A, P329G mutations in EU numbering). In some embodiments, the Fc region has a mutation at position 297 (e.g., N297A) according to EU numbering. In some embodiments, the mutated human IgG1 Fc region comprises an amino acid sequence that is at least 80%, 85%, 90%, 95%, 96%, 97%, 98%, 99%, or 100% identical to SEQ ID NO: 76.

In some embodiments, after being administered to a subject after 6 hours, 12 hours, 24 hours, 36 hours, 48 hours, 60 hours, or 72 hours, concentration of the antibody or antigen binding fragment thereof described herein in brain (e.g., whole brain or parenchyma) can be greater than 10%, 20%, 30%, 40%, 50%, 60%, 70%, or 80% of its concentration immediately (e.g., 0.5 hour) after administration. In some embodiments, after being administered to a subject after 6 hours, 12 hours, 24 hours, 36 hours, 48 hours, 60 hours, or 72 hours, concentration of the antibody or antigen binding fragment thereof described herein in brain (e.g., whole brain or parenchyma) can be at least 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 1000-fold, 2000-fold, 5000-fold, or 10000-fold of the concentration of a control antibody (e.g., hIgG1 or JR141-N), or the concentration in serum of the subject.

### Methods of Making Anti-TFR1 Antibodies

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein, e.g., TFR1. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites. In some embodiments, the heavy-chain antibody or antigen-binding fragment thereof described herein is obtained by immunizing any of the genetically modified animals (e.g., mice that are homozygous for heavy chain Mutant allele 3 genotype) described herein.

Humanized antibodies include antibodies having variable and constant regions derived from (or having the same amino acid sequence as those derived from) human germline immunoglobulin sequences of human immunoglobulin scaffold sequences. Humanized antibodies may include amino acid residues not encoded by human germline immunoglobulin sequences (e.g., mutations introduced by random or site-specific mutagenesis in vitro or by somatic mutation in vivo). Accordingly, “humanized” antibodies are chimeric antibodies wherein sequences from a non-human species are substituted by the corresponding human sequences.

Ordinarily, amino acid sequence variants of the human, humanized, or chimeric anti-TFR1 antibody will contain an amino acid sequence having at least 75%, 80%, 85%, 90%, 95%. 96%, 97%, 98%, or 99% percent identity with a sequence present in the VHH domain of the original antibody.

Identity or homology with respect to an original sequence is usually the percentage of amino acid residues present within the candidate sequence that are identical with a sequence present within the human, humanized, or chimeric anti-TFR1 antibody or fragment, after aligning the sequences and introducing gaps, if necessary, to achieve the maximum percent sequence identity, and not considering any conservative substitutions as part of the sequence identity.

Additional modifications to the anti-TFR1 antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. Wolff et al. (“Monoclonal antibody homodimers: enhanced antitumor activity in nude mice.” Cancer research 53.11 (1993): 2560-2565). Alternatively, an antibody can be engineered which has dual Fc regions.

In some embodiments, a covalent modification can be made to the anti-TFR1 antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody composition may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. In some embodiments, the promoter is a cytomegalovirus (CMV) promoter. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH can be used.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

In some embodiments, the antibody or antigen-binding fragment thereof is expressed in yeast, insect cells, or mammalian cells (e.g., CHO cells).

### Methods of Treatment and Diagnosis

The anti-TFR1 antibodies or antibody or antigen-binding fragments thereof of the present disclosure can be used for various therapeutic purposes. In one aspect, the disclosure provides methods for treating a brain disease (e.g., brain cancer, dementia, or Alzheimer's disease) in a subject, methods of identifying a subject having a brain disease (e.g., brain cancer, dementia, or Alzheimer's disease), methods of reducing the risk of developing a brain disease, or methods of reducing the risk of developing an additional symptoms in a subject. In some embodiments, the treatment can halt, slow, retard, or inhibit progression of a brain disease (e.g., brain cancer, dementia, or Alzheimer's disease). In some embodiments, the treatment can result in the reduction of in the number, severity, and/or duration of one or more symptoms of a brain disease (e.g., brain cancer, dementia, or Alzheimer's disease) in a subject.

In one aspect, the disclosure features methods that include administering a therapeutically effective amount of an antibody or antigen-binding fragment thereof disclosed herein to a subject in need thereof (e.g., a subject having, or identified or diagnosed as having, a brain disease).

In one aspect, the disclosure features methods that carry a therapeutic agent to cross the blood brain barrier. In some embodiments, the antibodies or antigen-binding fragments thereof as described herein are linked to the therapeutic agent. In some embodiments, the therapeutic agent is an antibody, an antigen binding fragment thereof, a small molecule, or an antibody-drug conjugate.

In some embodiments, the compositions and methods disclosed herein can be used for treatment of patients at risk for a brain disease (e.g., brain cancer, dementia, or Alzheimer's disease). Patients with a brain disease (e.g., brain cancer, dementia, or Alzheimer's disease) can be identified with various methods known in the art.

In some embodiments, the brain disease is a brain cancer.

In one aspect, the disclosure is related to methods of decreasing the rate of tumor growth, including contacting a tumor cell with an effective amount of a composition including the antibody or antigen-binding fragment thereof, or the antibody-drug conjugate described herein. In one aspect, the disclosure is related to methods of killing a tumor cell, including contacting a tumor cell with an effective amount of a composition including the antibody or antigen-binding fragment thereof, or the antibody-drug conjugate described herein.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment thereof is an amount sufficient to ameliorate, stop, stabilize, reverse, inhibit, slow and/or delay progression of a disease in a patient. As is understood in the art, an effective amount of an antibody or antigen binding fragment may vary, depending on, inter alia, patient history as well as other factors such as the type (and/or dosage) of antibody used.

Effective amounts and schedules for administering the antibodies, antibody-encoding polynucleotides, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, and/or compositions disclosed herein, the route of administration, the particular type of antibodies, antibody-encoding polynucleotides, antigen binding fragments, and/or compositions disclosed herein used and other drugs being administered to the mammal. Guidance in selecting appropriate doses for antibody or antigen binding fragment can be found in the literature on therapeutic uses of antibodies and antigen binding fragments, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of an antibody is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, or pharmaceutical composition (e.g., any of the antibodies, antigen-binding fragments, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody or antigen-binding fragment and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of the disease). As described herein, a skilled medical professional can also change the identity and number (e.g., increase or decrease) of antibodies or antigen-binding antibody fragments (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-PD1 antibody, an anti-PD-L1 antibody, an anti-LAG-3 antibody, an anti-TIGIT antibody, an anti-BTLA antibody, an anti-CTLA-4 antibody, or an anti-GITR antibody.

### Pharmaceutical Compositions and Routes of Administration

Also provided herein are pharmaceutical compositions that contain at least one (e.g., one, two, three, or four) of the antibodies or antigen-binding fragments described herein. Two or more (e.g., two, three, or four) of any of the antibodies or antigen-binding fragments described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies or antigen-binding fragments described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for case of administration and uniformity of dosage).

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease in a subject in a subject, or a subject identified as being at risk of developing the disease, decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof for various uses as described herein.

### Additional Embodiments

It is to be understood that while the invention has been described in conjunction with the detailed description thereof, the description is intended to illustrate and not limit the scope of the invention, which is defined by the scope of the claims. Other aspects, advantages, and modifications are within the scope of the claims. Also provided are additional embodiments.

Embodiment 1 is a genetically modified rodent comprising a modified immunoglobulin heavy chain locus, wherein the modified immunoglobulin heavy chain locus comprises an IgG constant region gene, wherein the IgG constant region gene encodes an IgG heavy chain constant region lacking a CH1 domain, wherein the genetically modified rodent expresses a heavy-chain antibody.

Embodiment 2 is the genetically modified rodent of embodiment 1, wherein the rodent comprises exactly one IgG constant region gene.

Embodiment 3 is the genetically modified rodent of embodiment 1 or 2, wherein the IgG heavy chain constant region gene is IGHG1.

Embodiment 4 is the genetically modified rodent of any one of embodiments 1-3, wherein the IgG heavy chain constant region comprises or consists of a CH2 domain and a CH3 domain, and optionally a hinge region.

Embodiment 5 is a genetically modified rodent whose genome comprises a germline genetic modification comprising a deletion of IGHG3, IGHG2b, and IGHG2c genes and a deletion of the CH1 exon of IGHG1 gene at a rodent immunoglobulin heavy chain gene locus.

Embodiment 6 is the rodent of embodiment 5, wherein the germline genetic modification further comprises a deletion of rodent IGHE gene at the rodent immunoglobulin heavy chain gene locus.

Embodiment 7 is the rodent of embodiment 5 or 6, wherein the genetic modification further comprises a deletion of rodent Sγ2b, Sγ2c, and Se switch regions at the rodent immunoglobulin heavy chain gene locus.

Embodiment 8 is the rodent of any one of embodiments 5-7, wherein the modified immunoglobulin heavy chain gene locus comprises a modified IGHG1 gene lacking a sequence encoding a CH1 domain, wherein the modified IGHG1 gene comprises a sequence that is at least 80%, 90%, 95% or 99% identical to SEQ ID NO: 1.

Embodiment 9 is the rodent of any one of embodiments 5-8, wherein the genetic modification further comprises a deletion of rodent Sγ3 switch region at the rodent immunoglobulin heavy chain gene locus.

Embodiment 10 is the rodent of any one of embodiments 5-9, wherein the rodent's genome comprises rodent Sμ, Sγ1, Sα switch regions, the modified rodent IGHG1 gene lacking a sequence encoding a CH1 domain, and rodent IGHM, IGHδ, IGHA genes.

Embodiment 11 is the rodent of any one of embodiments 5-9, wherein the genetic modification further comprises a deletion of rodent IGHM and IGHδ genes at the rodent immunoglobulin heavy chain gene locus.

Embodiment 12 is the rodent of any one of embodiments 5-9 and 11, wherein the rodent genome comprises rodent Sμ, Sγ1, Sα switch regions, the modified IGHG1 gene lacking a sequence encoding a CH1 domain, and the rodent IGHA gene.

Embodiment 13 is the rodent of embodiment 12, wherein the Su and Sγ1 switch regions are linked with a sequence that is at least 80%, 90%, 95% or 99% identical to SEQ ID NO: 8.

Embodiment 14 is the rodent of any one of embodiments 5-9, wherein the genetic modification further comprises a deletion of the CH1 coding sequence of IGHM gene at the rodent immunoglobulin heavy chain gene locus.

Embodiment 15 is the rodent of any one of embodiments 5-9 and 14, wherein the rodent's genome comprises rodent Sμ, Sγ1, Sα switch regions, a modified rodent IGHM gene lacking a sequence encoding a CH1 domain, the modified IGHG1 gene lacking a sequence encoding a CH1 domain, and rodent IGHδ, IGHA genes.

Embodiment 16 is the rodent of embodiment 15, wherein the Su switch region and the modified IGHM gene are linked with a sequence that is at least 80%, 90%, 95% or 99% identical to SEQ ID NO: 10.

Embodiment 17 is the rodent of any one of embodiments 5-9, wherein the genetic modification further comprises a deletion of the CH1 exon of IGHM gene and a deletion of IGHδ gene at the rodent immunoglobulin heavy chain gene locus.

Embodiment 18 is the rodent of any one of embodiments 5-9 and 17, wherein the rodent's genome comprises rodent Sμ, Sγ1, Sα switch regions, a modified IGHM gene lacking a sequence encoding a CH1 domain, the modified IGHG1 gene lacking a sequence encoding a CH1 domain, and a rodent IGHA gene.

Embodiment 19 is the rodent of any one of embodiments 5-9, wherein the genetic modification further comprises a deletion of the CH1 exon of IGHM gene and a deletion of the CH1 coding sequence of IGHδ gene at the rodent immunoglobulin heavy chain gene locus.

Embodiment 20 is the rodent of any one of embodiments 5-9 and 19, wherein the rodent's genome comprises rodent Sμ, Sγ1, Sα switch regions, a modified IGHM gene lacking a sequence encoding a CH1 domain, a modified IGHδ gene lacking a sequence encoding a CH1 domain, the modified IGHG1 gene lacking a sequence encoding a CH1 domain, and a rodent IGHA gene.

Embodiment 21 is the rodent of embodiment 19 or 20, wherein the modified IGHM gene are linked with a sequence that is at least 80%, 90%, 95% or 99% identical to SEQ ID NO: 10, and the modified IGHδ gene comprises a sequence that is at least 80%, 90%, 95%, or 99% identical to SEQ ID NO: 41.

Embodiment 22 is the rodent of any one of embodiments 14-21, wherein the modified IGHM gene comprises a sequence that is at least 80%, 90%, 95%, or 99% identical to SEQ ID NO: 13.

Embodiment 23 is the rodent of any one of embodiments 5-8, wherein the genetic modification further comprises a deletion of rodent Sγ1 switch region at the rodent immunoglobulin heavy chain gene locus.

Embodiment 24 is the rodent of any one of embodiments 5-8 and 23, wherein the rodent's genome comprises rodent Sμ, Sγ3, Sα switch regions, the modified IGHG1 gene lacking a sequence encoding a CH1 domain, and rodent IGHM, IGHδ, IGHA genes.

Embodiment 25 is the rodent of any one of embodiments 5-8 and 23, wherein the genetic modification further comprises a deletion of rodent Sγ3 switch region at the rodent immunoglobulin heavy chain gene locus.

Embodiment 26 is the rodent of any one of embodiments 5-8, 23 and 25, wherein the genetic modification further comprises a deletion of rodent IGHM and IGHδ genes at the rodent immunoglobulin heavy chain gene locus.

Embodiment 27 is the rodent of any one of embodiments 5-8, 23, 25 or 26, wherein the rodent's genome comprises rodent Sμ, Sα switch regions, the modified IGHG1 gene lacking a sequence encoding a CH1 domain, and the rodent IGHA gene.

Embodiment 28 is the rodent of embodiment 27, wherein the Su switch region and the modified IGHG1 gene are linked with a sequence that is at least 80%, 90%, 95%, or 99% identical to SEQ ID NO: 9.

Embodiment 29 is the rodent of any one of embodiments 5-10, 23 and 24, wherein the modified genome comprises a functional IGHM gene.

### EXAMPLES

The invention is further described in the following examples, which do not limit the scope of the invention described in the claims.

### Example 1: Overview

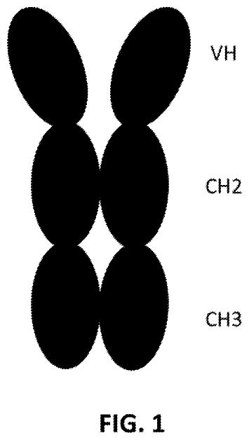

Immunoglobulin heavy chain locus of non-human animals was modified by gene editing. For example, in order to obtain mice that can express heavy-chain antibodies, the immunoglobulin heavy chain constant region locus within mouse chromosome 12 was modified. The genetically-modified mice can express heavy-chain antibodies as shown in FIG. 1. Furthermore, all endogenous VDJ sequences in the heavy chain variable region locus on chromosome 12 was replaced by the human VDJ sequences, so that the variable regions in the heavy-chain antibodies expressed by the mice had fully humanized sequences. In some experiments, the genetic modification on immunoglobulin heavy chain constant region locus was performed on mice with human VDJ sequences.

### Example 2: Modification of Mouse Immunoglobulin Heavy Chain Constant Region Locus

As shown in FIG. 2, the mouse (C57BL/6) immunoglobulin constant region genes include (as shown in the following order): immunoglobulin heavy constant mu (IGHM, or Cμ), immunoglobulin heavy constant delta (IGHδ, or Cδ), immunoglobulin heavy constant gamma 3 (IGHG3, or Cγ3), immunoglobulin heavy constant gamma 1 (IGHG1, or Cγ1), immunoglobulin heavy constant gamma 2b (IGHG2b, or Cγ2b), immunoglobulin heavy constant gamma 2c (IGHG2c, or Cγ2c), immunoglobulin heavy constant epsilon (IGHE, or Cε), and immunoglobulin heavy constant alpha (IGHA, or Cα) genes. As shown in FIG. 3, switch regions (e.g., Sμ, Sγ3, Sγ1, Sγ2b, Sγ2c, Sε, and Sα) and their respective promoters are located upstream of the corresponding constant region genes. Particularly, the Cγ1 gene includes sequences that encode, from N-terminus to C-terminus, CH1, H (hinge), CH2, CH3, M1 and M2 regions of IgG1.

The mouse immunoglobulin heavy chain constant region locus was modified by various methods discussed as follows. The experiments were performed in mice with fully humanized VDJ sequences. Details of the VDJ region humanized mice can be found, e.g., in WO2020169022A1 and US20200390073A1; each of which is incorporated herein by reference in its entirety.

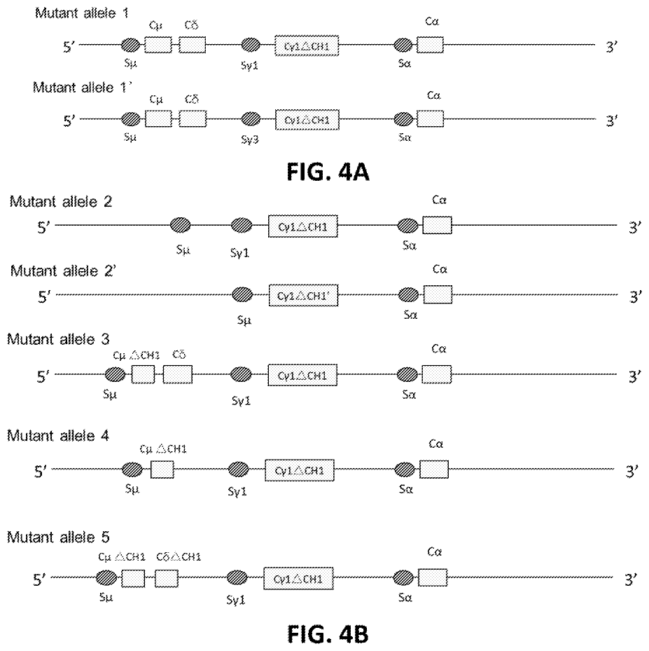

In order to obtain mice expressing only heavy-chain antibodies, the Cγ3, Cγ1, Cγ2b, Cγ2c, and Cε gene loci were modified to only keep a truncated Cγ1 sequence without the CH1 coding region (Cγ1ΔCH1), to obtain a mutated allele shown as Mutant allele 1 or Mutant allele 1′ in FIG. 4A. On the basis of Mutant allele 1, different modifications were made to the switch region (e.g., Su and Sγ1), Cμ and/or Cδ sequences. For example, the entire Cμ and Cδ sequences were knocked out, and the resultant alleles are shown as Mutant allele 2 and Mutant allele 2′ (further missing Sγ1). Alternatively, the region being knocked out was selected from: the CH1 coding region of Cμ (with resultant allele shown as Mutant allele 3); the CH1 coding region of Cμ and the entire Co (with resultant allele shown as Mutant allele 4); and both the CH1 coding region of Cμ and the CH1 coding region of Co (with resultant alleles shown as Mutant allele 5).

### Example 3: Construction of Targeting Vectors

Targeting vectors, e.g., V1, V2, V3, V4, V5, V6, and V7 were used in the modifications.

### Vector V1

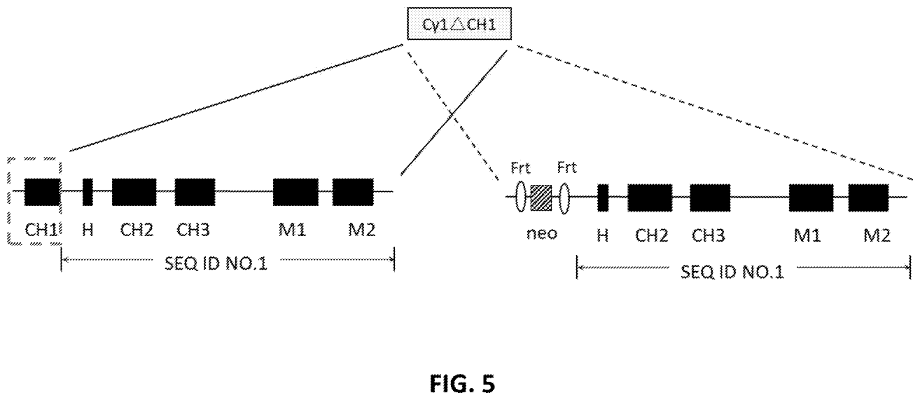

Vector V1 included the Cγ1ΔCH1 knock-in sequence (SEQ ID NO: 1) as shown in FIG. 5. The Cγ1ΔCH1 sequence did not have the coding sequence of CH1. The CH1 coding region was either deleted or replaced with a Neo cassette. The Neo cassette includes a Neo gene sequence flanked by two Frt (or LoxP) sequences. Flp transgenic mice were bred with the mice carrying the Neo cassette to remove the cassette.

The targeting vector V1 included the following feature sequences in a 5′ to 3′ order: an upstream homologous arm (5′ homologous arm), the mouse Sγ1 promoter sequence, the mouse Sγ1, the Cγ1ΔCH1 sequence, and a downstream homologous arm (3′ homologous arm). The targeting vector can further include an antibiotic resistance gene for positive clone screening (e.g., neomycin phosphotransferase gene, or Neo), and two Frt recombination sites flanking the antibiotic resistance gene. In addition, a coding gene with a negative selectable marker (a gene encoding diphtheria toxin A subunit (DTA)) can also be inserted to the targeting vector.

A 16076 bp sequence comprising the mouse Sγ1 promoter, the mouse Sγ1, and the Cγ1ΔCH1 knock-in sequence (SEQ ID NO: 1) was cloned from mouse bacterial artificial chromosome (RP23-38K22 or RP23-265P18). The following two sets of primer pairs were used to obtain a sequence comprising mouse Sγ1 promoter, the mouse Sγ1, and the Cγ1ΔCH1 knock-in sequence for the construction of the V1 vector. The sequence amplified from V1-F1 and V1-R1 and the sequence amplified from V1-F2 and V1-R2 were then ligated.

V1-F1 (SEQ ID NO: 2):
5′-GTGGTTCTGGCTACAAGATAGAGCTCTGTCAATGATGTTTGCAGAGA
CTACA-3′
V1-R1 (SEQ ID NO: 3):
5′-CTCCCTATACGTCCTCTCACCTACAAGAAAAAGTATATGTGATTACA
CTGTCAGACAG-3′
V1-F2 (SEQ ID NO: 4):
5′-GTGTAATCACATATACTTTTTCTTGTAGGTGAGAGGACGTATAGGGA
GGAGGGGTTC-3′
V1-R2 (SEQ ID NO: 5):
5′-CGTCTAGTCCTTGCCCACGTGTCGACCCCATAGGGAGGACAGACTGA
GG-3′

As shown in FIG. 6, the V1 vector was used to replace a 100883 bp sequence (nucleic acids 113232142 to 113333024 of NCBI Reference Sequence NC_000078.7) of the mouse heavy chain constant region locus (from Sγ3 to Cε) in a single step.

### Vector V2

As shown in FIG. 7, the V2 vector was used to replace a 92859 bp sequence (nucleic acids 113232142 to 113325000 of NCBI Reference Sequence NC_000078.7) of the mouse heavy chain constant region (spanning from Cγ3 to Cα) in a single step.

The method also included using a pair of primers to clone from mouse bacterial artificial chromosome (BAC) to obtain a knock-in sequence comprising the Cγ1ΔCH1 knock-in sequence (SEO ID NO: 1). The following primers were used:

V2-F1 (SEQ ID NO: 6):
5′-TCTGAACTACTTCGTCGACGTGAGAGGACGTATAGGGAGGAGGG-3′
V2-R1 (SEQ ID NO: 7):
5′-CACGTGGATCCGCGGCCGCCCATAGGGAGGACAGACTGAGGAC-3′

### Vector V3

As shown in FIG. 8, the V3 vector was used to knock out a total of 16434 bp nucleotides from the chromosome including Cμ and Cδ. The linkage sequence between Su and Sγ1 in the recombined Mutant allele 2 is shown in SEQ ID NO: 8.

### Vector V4

As shown in FIG. 9, the targeting vector V4 included the following feature sequences in a 5′ to 3′ order: an upstream homologous arm (5′ homologous arm), a portion of the mouse Sμ, and a downstream homologous arm (3′ homologous arm). The targeting vector further included an antibiotic resistance gene for positive clone screening (neomycin phosphotransferase gene, or Neo), and two Frt recombination sites flanking the antibiotic resistance gene. In addition, a coding gene with a negative selectable marker (a gene encoding diphtheria toxin A subunit (DTA)) was also inserted to the targeting vector. Specifically, Su was directly linked to Cγ1ΔCH1′ in the recombined Mutant allele 2′ (i.e., there was no other switch region or immunoglobulin gene sequences in between). The linkage sequence is shown in SEQ ID NO: 9.

### Vector V5

As shown in FIG. 10, the V5 vector was used to knock out the CH1 coding sequence in Cμ. Upon deletion of the CH1 coding sequence in Cμ, the linkage sequence is shown in SEQ ID NO: 10.

### Vector V6

As shown in FIG. 11, the targeting vector V6 included the following feature sequences in a 5′ to 3′ order: an upstream homologous arm (5′ homologous arm), a portion of the mouse Su region, a mouse CμΔCH1 sequence and a downstream homologous arm (3′ homologous arm). The targeting vector further included an antibiotic resistance gene for positive clone screening (neomycin phosphotransferase gene, or Neo), and two Frt recombination sites flanking the antibiotic resistance gene. In addition, a coding gene with a negative selectable marker (a gene encoding diphtheria toxin A subunit (DTA)) was also inserted to the targeting vector.

A sequence including the mouse CμΔCH1 sequence were cloned from mouse somatic cells. The following primer pair was used to obtain a sequence including the mouse CμΔCH1 (or CμΔCH1 knock-in sequence; SEQ ID NO: 13) for the construction of the V6 vector.

V6-F1 (SEQ ID NO: 11):
5′-ATCCCTCTCTGGTCCTAACCAAACCCTCCCAGCAGGGGTG-3′
V6-R1 (SEQ ID NO: 12):
5′-TTGACCCATCTCAGTTTACATGGTGAATGACTACAATATATCTGGAA
TTTGG-3′

### Vector V7

As shown in FIG. 12, the targeting vector V7 included the following feature sequences in a 5′ to 3′ order: an upstream homologous arm (5′ homologous arm), a CμΔCH1 sequence, a CδΔCH1 sequence (or CδΔCH1 knock-in sequence; SEQ ID NO: 41) and a downstream homologous arm (3′ homologous arm). The targeting vector further included an antibiotic resistance gene for positive clone screening (neomycin phosphotransferase gene, or Neo), and two Frt recombination sites flanking the antibiotic resistance gene. In addition, a coding gene with a negative selectable marker (a gene encoding diphtheria toxin A subunit (DTA)) was also inserted to the targeting vector.

A sequence including the mouse Su switch region, the CμΔCH1 sequence and the CδΔCH1 sequence were cloned from mouse genome. The following primer pair was used to obtain a knock-in sequence including the mouse CμΔCH1 and nucleotides upstream of CδΔCH1 for the construction of the V7 vector.

V7-F1 (SEQ ID NO: 14):
5′-ATCCCTCTCTGGTCCTAACCAAACCCTCCCAGCAGGGGTG -3′
V7-R1 (SEQ ID NO: 15):
5′-TTCTGCATGGTCCAGGGATTGATCAGACAGATAGTGAAGTTCTGAGG
ACA-3′

### Example 4: Verification of Genetic Modifications

### Mutant Allele 1

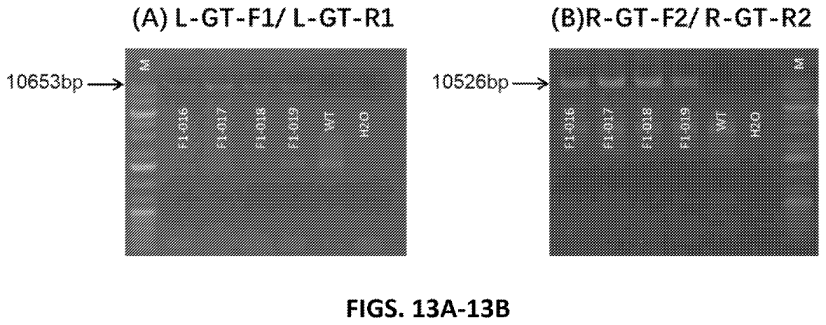

PCR and Southern Blot were used to detect the genotype of Mutant Allele 1. First, positive clone cells were identified by PCR using two sets of primer pairs L-GT-F1/L-GT-R1 and R-GT-F2/R-GT-R2. Exemplary results are shown in FIGS. 13A-13B, respectively. Sequences of the primers are shown in the table below.

TABLE 7

| | | Fragment |
|---|---|---|
| Primer | Sequence (5′ to 3′) | size (bp) |
| L-GT-F1 | TAGGAAGTGGCTCAGAAGTCTGCCT | Mut: 10653 bp |
| | (SEQ ID NO: 16) | |
| L-GT-R1 | TCTCCAGCCCCATTTATCCCAGCTA | |
| | (SEQ ID NO: 17) | |
| R-GT-F2 | TGGGATAGCTATGTGGGGAGACCAG | Mut: 10526 bp |
| | (SEQ ID NO: 18) | |
| R-GT-R2 | AGTTCTGTCATCAGGCCAACTACGC | |
| | (SEQ ID NO: 19) | |

Next, the positive clones were verified by Southern Blot (digested with BclI, ScaI, XmnI, and BglII, respectively, followed by hybridization with 4 corresponding probes) to screen out correct positive clone cells. The Southern Blot detection strategy (including restriction enzymes, probes, and target fragment sizes) and the probe primers are shown in the tables below.

TABLE 8

| Restriction enzyme | Probe | WT size | Targeted size |
|---|---|---|---|
| BclI | LR Probe | 6.1 kb | 18.4 kb |
| ScaI | 3′ Probe | 9.7 kb | 12.8 kb |
| XmnI | A Probe | 9.7 kb | 16.6 kb |
| BglII | 5′ Probe | 7.2 kb | 4.3 kb |

TABLE 9

| Probe | Sequence (5′ -3′) |
|---|---|
| LR | TCCTTGCCCCTCAGTCAGTCTTGA (SEQ ID NO: 20) |
| Probe | TCCTCCATGGCTTGGTGCCTTCT (SEQ ID NO: 21) |
| 3′ | GCCAGCTCTTGAGGACCTGCTCC (SEQ ID NO: 22) |
| Probe | TGGCTCAGACAAGGAGAGCAAGACC (SEQ ID NO: 23) |
| A | ATTAGCCTGCCTAAACCAACCAGGC (SEQ ID NO: 24) |
| Probe | TCAATACCTGGGCCACCCTCTAACA (SEQ ID NO: 25) |
| 5′ | GAAGTCTGCCTGACCCTCTACATG (SEQ ID NO: 26) |
| Probe | CACAGATGTGTAATACATGTACTTGC (SEQ ID NO: 27) |

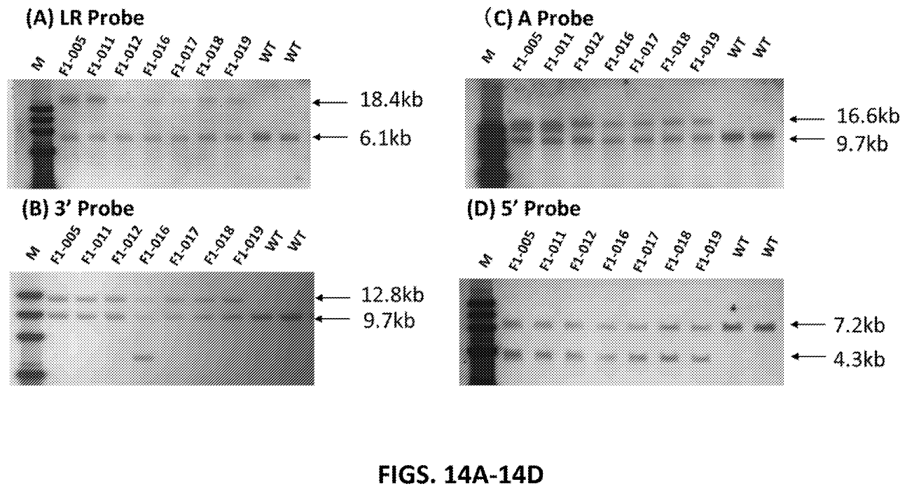

Exemplary detection results are shown in FIGS. 14A-14D. According to the PCR and Southern Blot results, mice numbered F1-012, F1-017, F1-018 and F1-019 were identified as Mutant Allele 1 positive heterozygous mice. No random insertion in Mutant Allele 1 was detected.

### Mutant Allele 2

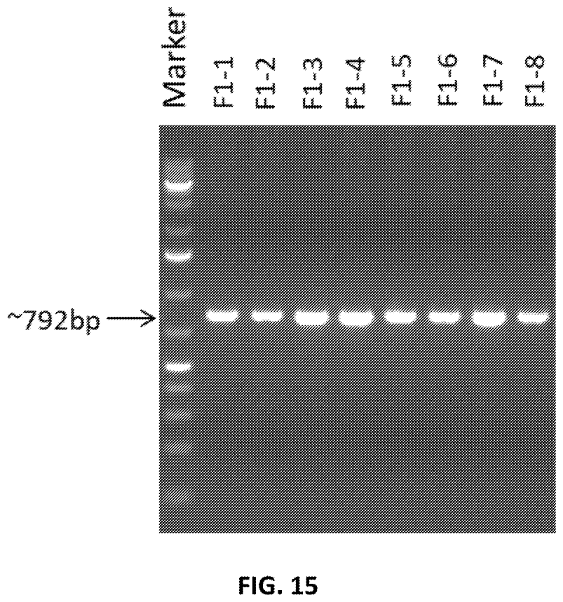

Primers DE-F1 and DE-R1 were used to confirm knock-out of the sequence from Cμ to Cδ in Mutant Allele 2. The detection result is shown in FIG. 15. Mice numbered F1-1, F1-2, F1-3, F1-4, F1-5, F1-6, F1-7 and F1-8 were identified as positive heterozygous mice. Sequences of the primers are shown in the table below.

TABLE 10

| | | Fragment |
|---|---|---|
| Primer | Sequence (5′ to 3′) | size (bp) |
| DE-F1 | TGTGTGCTGATACAGAAGCCTGAGC | Mut: ~792 bp |
| | (SEQ ID NO: 28) | |
| DE-R1 | TCCCATCACCCTGTGTTATCATCTGT | |
| | (SEQ ID NO: 29) | |

### Mutant Allele 2′

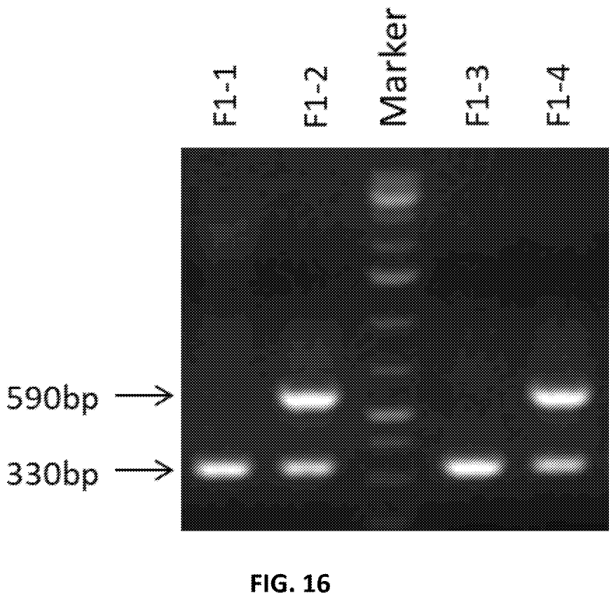

Primers GT-Mut-F, GT-Mut-R and GT-WT-R were used to confirm knock-out of the sequence from Cμ to Sγ1 in Mutant Allele 2′. The detection result is shown in FIG. 16. Mice numbered F1-2, and F1-4 were identified as positive heterozygous mice. Sequences of the primers are shown in the table below.

TABLE 11

| | | Fragment |
|---|---|---|
| Primer | Sequence (5′ to 3′) | size (bp) |
| GT-Mut-F | ACCATATCTAGCCCTGCCTGCCTTA | WT: 330 bp |
| | (SEQ ID NO: 30) | Mut: 590 bp |
| GT-Mut-R | TCAATACCTGGGCCACCCTCTAACA | |
| | (SEQ ID NO: 31) | |
| GT-WT-R | ACTTGCCCCCTGTCCTCAGTG | |
| | (SEQ ID NO: 32) | |

### Mutant Allele 3

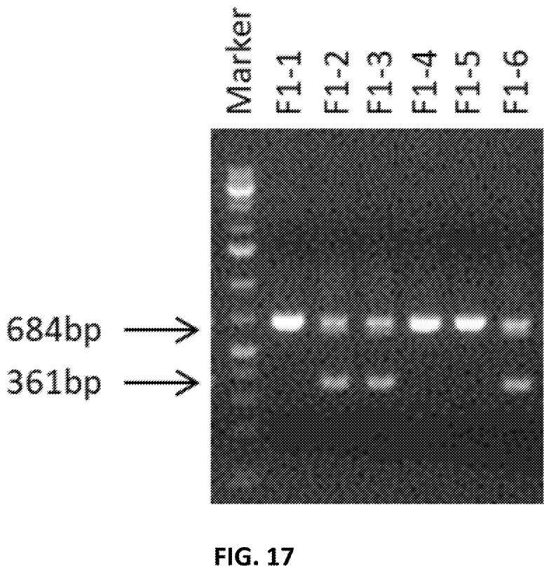

Primers GT-3F and GT-3R were used to confirm knock-out of the CH1 coding sequence of Cμ in Mutant Allele 3. The detection result is shown in FIG. 17. Mice numbered F1-2, F1-3 and F1-6 were identified as positive heterozygous mice. Sequences of the primers are shown in the table below.

TABLE 12

| | | Fragment |
|---|---|---|
| Primer | Sequence (5′ to 3′) | size (bp) |
| GT-3F | GTGGCTAGAAGGCAGCTCCA | Mut: 361 bp |
| | (SEQ ID NO: 33) | WT: 684 bp |
| GT-3R | CAGAGAAGCCATCCCGTGGT | |
| | (SEQ ID NO: 34) | |

### Mutant Allele 4

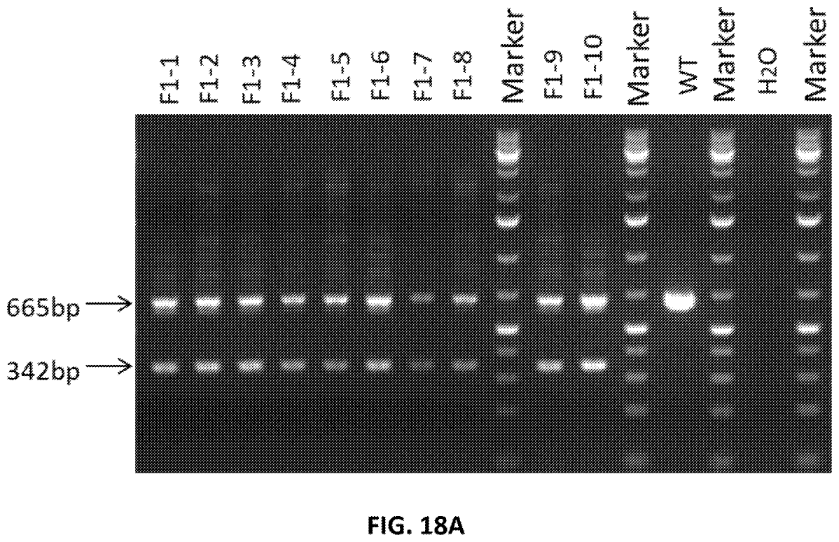

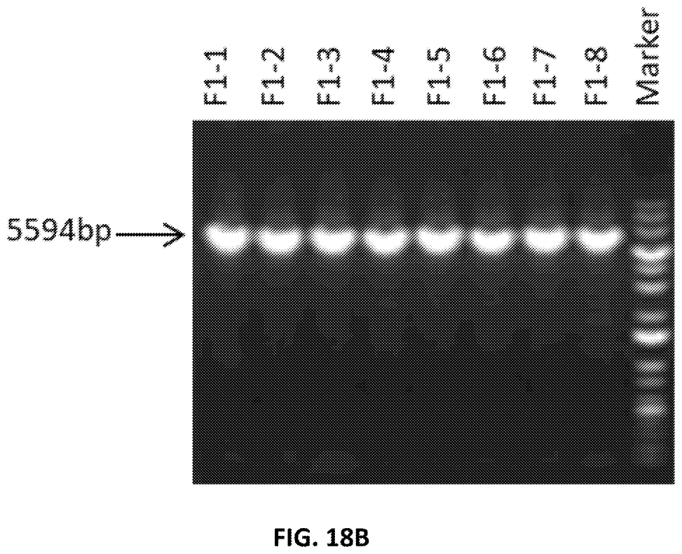

Primers Mut-F and Mut-R were used to confirm the sequence of CμΔCH1 in Mutant Allele 4. Primers F4 and R4 were used to confirm the absence of Co in Mutant Allele 4. The detection results are shown in FIGS. 18A-18B, respectively. Mice numbered F1-1, F1-2, F1-3, F1-4, F1-5, F1-6, F1-7, F1-8, F1-9 and F1-10 were identified as positive heterozygous mice. Sequences of the primers are shown in the table below.

TABLE 13

| | | Fragment |
|---|---|---|
| Primer | Sequence (5′ to 3′) | size (bp) |
| Mut-F | TACCATATCTAGCCCTGCCTGCCTT | Mut: 342 bp |
| | (SEQ ID NO: 35) | WT: 665 bp |
| Mut-R | TTCGTGGCCTCGCAGATGAGTTTAG | |
| | (SEQ ID NO: 36) | |
| F4 | GCCTCTTTGGGCTCATATCCTGGAC | Mut: 5594 bp |
| | (SEQ ID NO: 39) | |
| R4 | GAGTGTGTCACATGACTCCACTGGG | |
| | (SEQ ID NO: 40) | |

### Mutant Allele 5

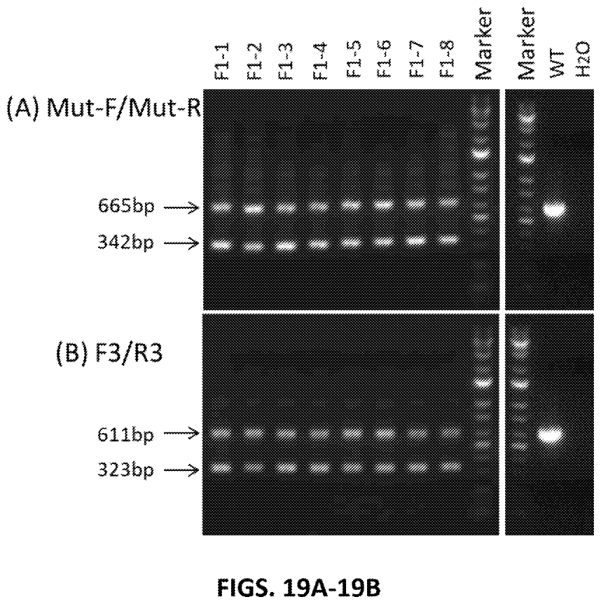

Primers Mut-F and Mut-R were used to confirm the sequence of CμΔCH1, and primers F3 and R3 were used to confirm the sequence of CδΔCH1 in Mutant Allele 5. The detection results are shown in FIGS. 19A-19B, respectively. Mice numbered F1-1, F1-2, F1-3, F1-4, F1-5, F1-6, F1-7 and F1-8 were identified as positive heterozygous mice. Sequences of the primers are shown in the table below.

TABLE 14

| | | Fragment |
|---|---|---|
| Primer | Sequence (5′ to 3′) | size (bp) |
| Mut-F | TACCATATCTAGCCCTGCCTGCCTT | Mut: 342 bp |
| | (SEQ ID NO: 35) | WT: 665 bp |
| Mut-R | TTCGTGGCCTCGCAGATGAGTTTAG | |
| | (SEQ ID NO: 36) | |
| F3 | CAGACCTCACCTTTGTCCCCAGTTC | Mut: 323 bp |
| | (SEQ ID NO: 37) | WT: 611 bp |
| R3 | GGTACTGCTATGGAAGGGACCATGC | |
| | (SEQ ID NO: 38) | |

### Example 5: Modification of Mouse Immunoglobulin Heavy Chain Variable Region Locus

Experiments were performed to introduce human immunoglobulin genes into the mouse genome to produce mice expressing humanized antibodies. FIG. 20 shows the methods of making the humanized mice. The methods first involve modifying the human immunoglobulin region on the human chromosome. The modified human chromosomes were then introduced into the mouse recipient cell.

Sequences of the mouse immunoglobulin variable region locus were replaced with sequences of the human immunoglobulin variable region locus by direct replacement (e.g., homologous recombination, or Cre-mediated recombination). In some cases, the human immunoglobulin variable region genes can be introduced into the mouse genome by a stepwise approach. Then, the recipient cells were screened for the correct replacement. The cells were then injected to blastocysts to prepare chimeric mice. Subsequent breeding was performed to obtain mice containing human or humanized immunoglobulin variable region locus sequences.

The immunoglobulin heavy chain locus is located on mouse chromosome 12. Two recombination sites were introduced on both sides of the immunoglobulin heavy chain variable region locus.

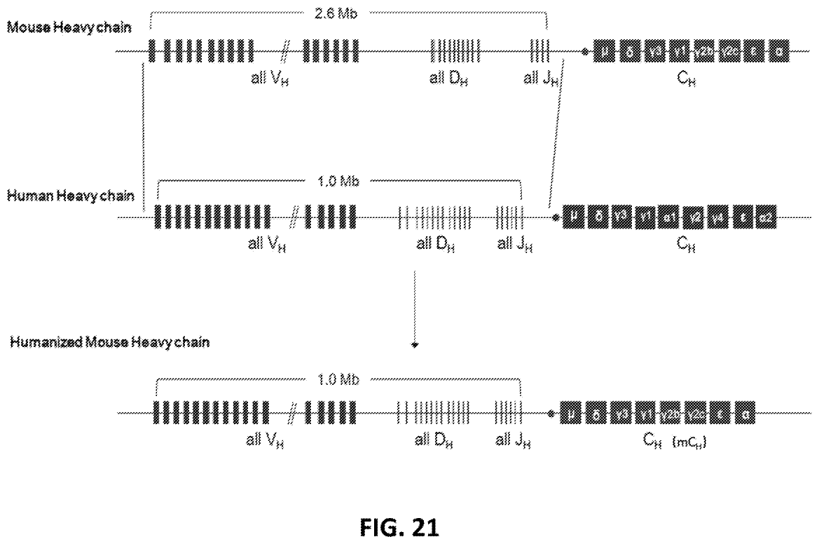

Experiments were also performed to generate a modified human chromosome. Two recombination sites were introduced on both sides of the variable region of the immunoglobulin heavy chain locus. The modified human chromosome was then introduced into mouse cell. The cells were then screened, and those containing only one human chromosome were selected. Cre recombinase then mediated the replacement of V, D, J regions on mouse chromosome with the V, D, J regions on human chromosome (FIG. 21).

The positive clone cells were injected into the blastocysts of BALB/c mice by microinjection. The embryo microinjection was carried out according to the method described, e.g., in A. Nagy, et al., “Manipulating the Mouse Embryo: A Laboratory Manual (Third Edition),” Cold Spring Harbor Laboratory Press, 2003. The injected fertilized eggs were then transferred to a culture medium for a short time culture, and then transplanted into the oviduct of the recipient mouse to produce the genetically-modified humanized mice (F0 generation). The mice were then bred with mice having C57BL/6 background. PCR analysis was performed on the DNA obtained from the tail of the mice. The mice were further bred with mice with BALB/c background several times (e.g., at least 5 times) to obtain heavy chain immunoglobulin locus humanized heterozygous mice with BALB/c background.

The heterozygous mice were then bred with each other to obtain homozygous mice. A detailed description of how to make immunoglobulin heavy chain locus humanized homozygous mice is provided in WO2020169022A1 and US20200390073A1; each of which is incorporated herein by reference in its entirety.

### Example 6: Modification of Mouse Immunoglobulin Light Chain Loci

### Kappa (κ) Light Chain Locus Knock-Out Mice

The immunoglobulin kappa light chain (κ) locus is located on mouse chromosome 6. The mouse chromosome 6 was modified by knocking out the entire sequence of the immunoglobulin kappa light chain variable region locus. Detailed knock-out methods can be found, e.g., in WO2020169022A1 and US20200390073A1; Zou, X., et al. “Subtle differences in antibody responses and hypermutation of λ light chains in mice with a disrupted χ constant region.” European Journal of Immunology 25.8 (1995): 2154-2162; Zou, Y. R., et al. “Gene targeting in the Ig kappa locus: efficient generation of lambda chain-expressing B cells, independent of gene rearrangements in Ig kappa.” The EMBO Journal 12.3 (1993): 811-820; Takeda, S., et al. “Deletion of the immunoglobulin kappa chain intron enhancer abolishes kappa chain gene rearrangement in cis but not lambda chain gene rearrangement in trans.” The EMBO Journal 12.6 (1993): 2329-2336; each of which is incorporated herein by reference in its entirety.

### Lambda (λ) Light Chain Locus Knock-Out Mice

The immunoglobulin lambda light chain (A) is located on mouse chromosome 16. The mouse chromosome 16 was modified by knocking out the entire sequence of the immunoglobulin lambda light chain variable region locus. Detailed knocking out methods can be found, e.g., in Zou, X., et al. “Block in development at the pre-B-II to immature B cell stage in mice without Igκ and Igλ light chain.” The Journal of Immunology 170.3 (2003): 1354-1361, which is incorporated herein by reference in its entirety.

The mice described herein can be bred with each other to obtain mice having human immunoglobulin heavy chain VDJ regions, modified mouse immunoglobulin heavy chain constant region locus (lacking CH1 coding region of Cγ1), and lacking all or part of mouse immunoglobulin light chain loci.

### Example 7: Heavy Chain Antibodies from Mice Carrying a Modified IgG1 Gene

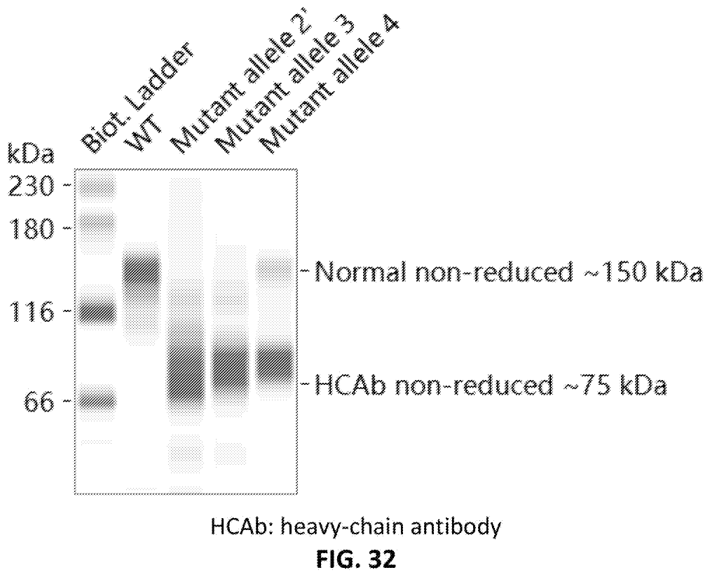

Mice identified above lacking CH1 coding region of Cγ1 (mice with Mutant allele 2′, Mutant allele 3, and Mutant allele 4 genotypes, respectively) and a wild-type (WT) mouse were bled to obtain serum samples. The serum samples were prepared for Western blotting analysis to identify any expressed IgG in the serum using an anti-mIgG1 antibody (Cat #: ab190481, Abcam). As shown in FIG. 32, the results revealed a mixture of bands: one band of about 75 kD (the expected size for a dimer IgG1 lacking a CH1 domain), and one band of about 150 kD (the expected size for a wild-type IgG). The results indicate that the mice produced using the methods described herein can express an IgG1 lacking a CH1 domain in the peripheral blood.

Mice can be immunized by injecting immunogens, and then screened for antibodies with specific binding by various methods (e.g., hybridoma, phage screening, single-cell technologies by 10×Genomics, or the Beacon® Optofluidic System). Preliminary results showed that mice prepared by the methods described herein can be used to obtain antibodies with high affinity, high diversity, good function (e.g., high endocytic activity), and good developability (e.g., high hydrophilicity and good thermal stability).

Mice with Mutant Allele 3 (Mut3)

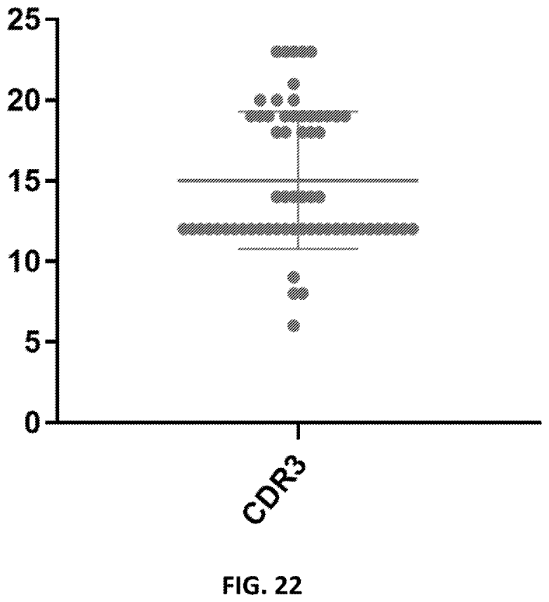

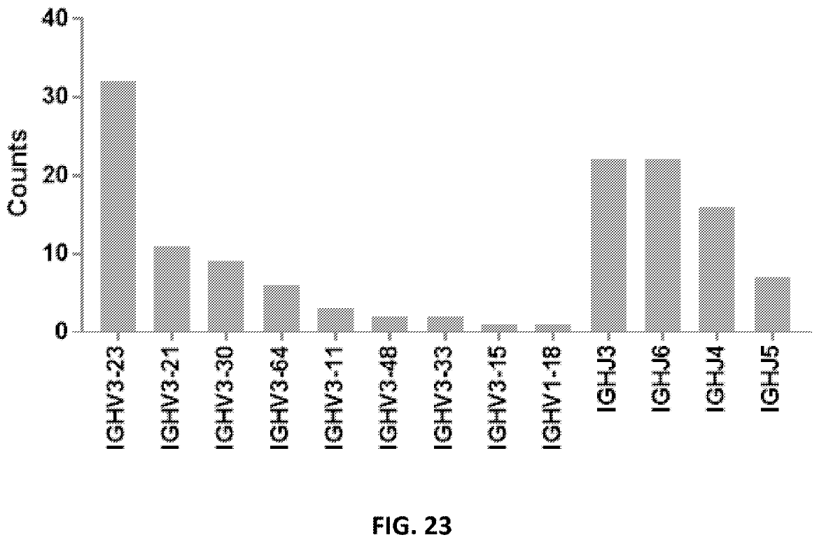

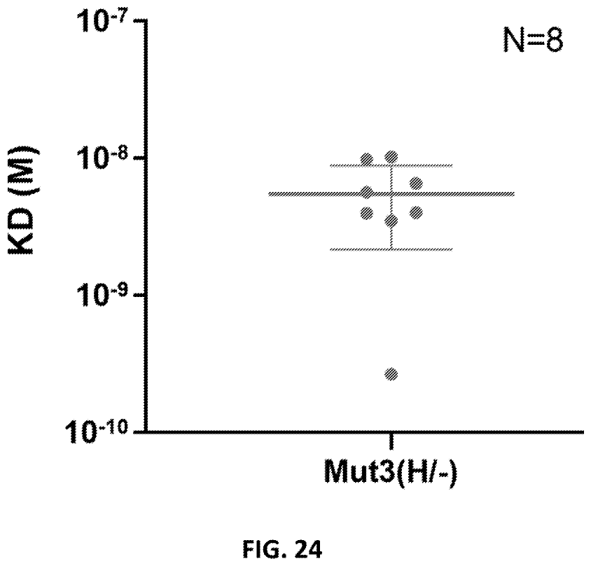

Humanized mice (heterozygous for heavy chain Mutant allele 3 genotype, kappa light chain locus knockout, and lambda light chain locus not knockout) were immunized with antigen A (5 mice). After 3 immunizations, serum titers increased by 10⁴ folds as detected by FACS. Beacon® Optofluidic System was then used to isolate the plasma cells that can produce antigen-specific monoclonal antibodies. Expression vector for each antibody was constructed and transfer into a host cell. The number of positive cells confirmed by FACS was 63. Further analysis of the antibody sequences showed that the CDR3 length of the heavy chain variable region (defined by IMGT) was between 6-23 (FIG. 22). In addition, 94% of the clones (59/63) had CDR3 lengths greater than or equal to 12, with a total of 35 unique CDR3 sequences. FIG. 23 shows the germline gene usage of the variable region genes. Further, the affinity of some antibodies was also tested. As shown in FIG. 24, the KD of these antibodies against antigen A reached 10⁻⁹ M, indicating good binding affinity.

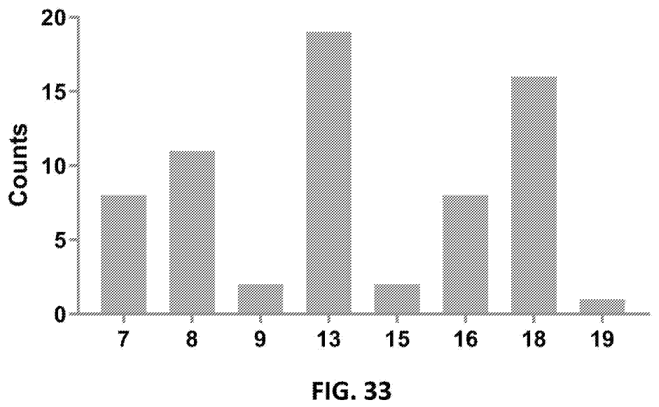

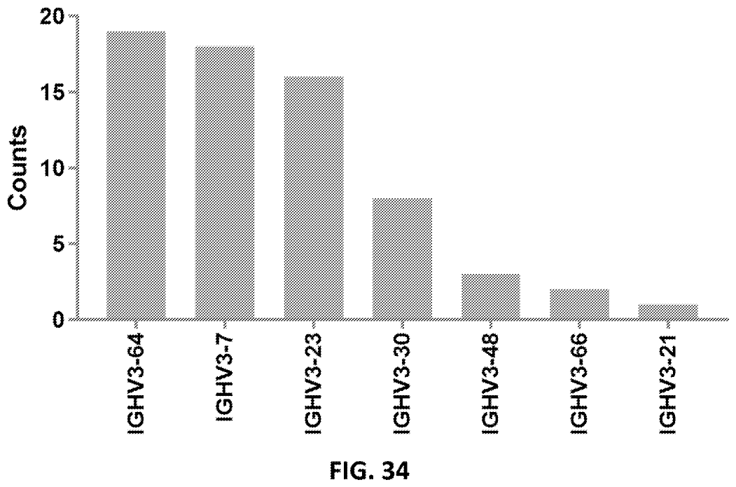

In another experiment, humanized mice (4 mice were homozygous for heavy chain Mutant allele 3 genotype, homozygous for the deletion of kappa light chain locus, and heterozygous for the deletion of lambda light chain locus; 3 mice were homozygous for heavy chain Mutant allele 3 genotype, homozygous for the deletion of kappa light chain locus, and homozygous for the deletion of lambda light chain locus) were immunized with human 4-1BB (Cat #: 41B-H5258, ACROBiosystems). After 4 immunizations, serum titers increased by 10⁴ folds as detected by FACS. The number of antigen-specific clones confirmed by FACS was 67. Further analysis of the antibody sequences showed that the CDR3 length of the heavy chain variable region (defined by IMGT) was between 7-19 (FIG. 33). FIG. 34 shows the germline gene usage of the variable region genes. Further, the affinity of some antibodies was also tested.

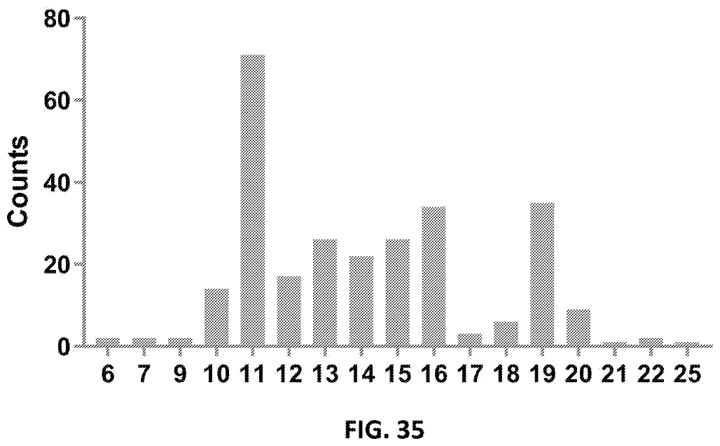

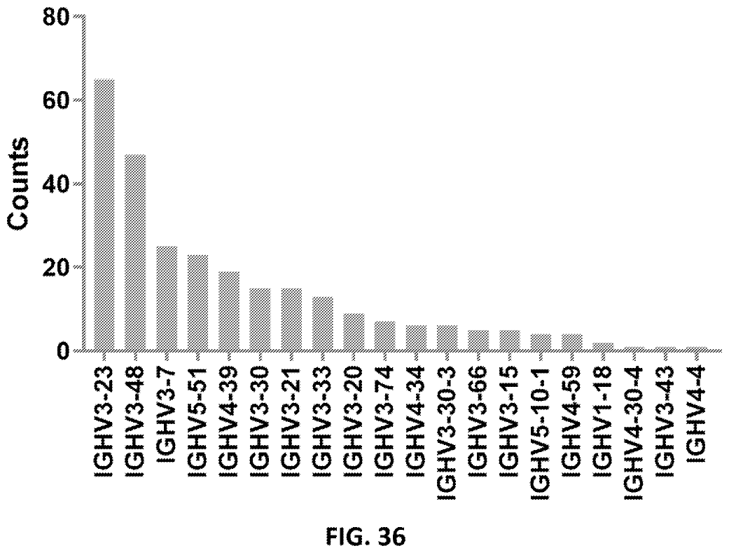

In another experiment, humanized mice (19 mice were homozygous for heavy chain Mutant allele 3 genotype, homozygous for the deletion of kappa light chain locus, and heterozygous for the deletion of lambda light chain locus) were immunized with antigen human CD3ED and cynomolgus CD3ED. After 4 immunizations, serum titers increased by 10⁵ folds as detected by FACS. The number of antigen-specific clones confirmed by FACS was 273. Further analysis of the antibody sequences showed that the CDR3 length of the heavy chain variable region (defined by IMGT) was between 6-25 (FIG. 35). FIG. 36 shows the germline gene usage of the variable region genes. Further, the affinity of some antibodies was also tested.

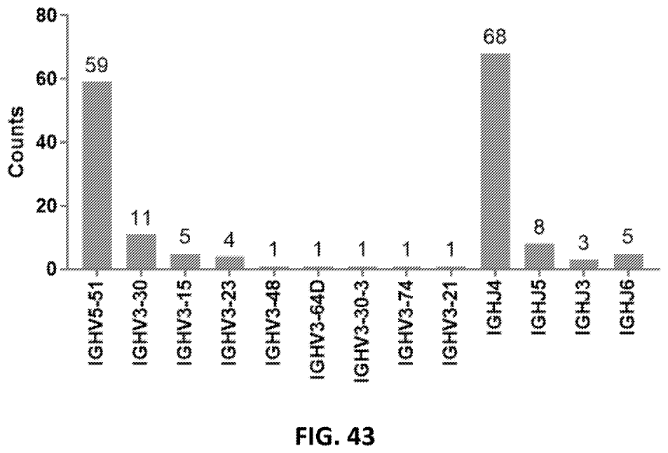

Humanized mice (homozygous for heavy chain Mutant Allele 3 genotype, kappa light chain locus and lambda light chain locus knockout) were immunized with antigen Human Serum Albumin (10 mice). Beacon® Optofluidic System was then used isolate the plasma cells that can produce antigen-specific monoclonal antibodies. The number of positive cells confirmed by FACS was 84. Further analysis of the antibody sequences showed that the CDR3 length of the heavy chain variable region (defined by IMGT) was between 12-17. FIG. 43 shows the germline gene usage of the variable region genes.

Mice with Mutant Allele 2 (Mut2)

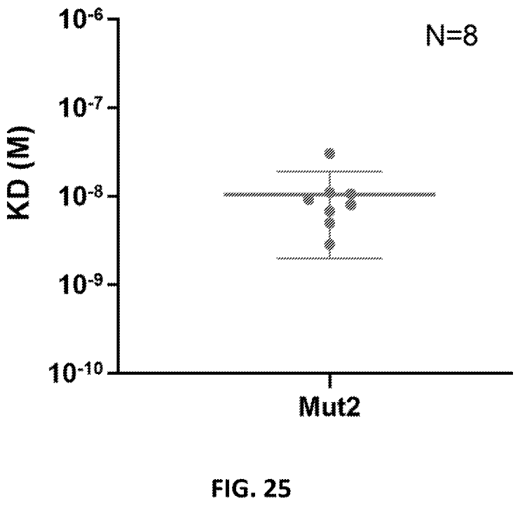

Humanized mice (homozygous for heavy chain Mutant Allele 2 genotype, kappa light chain locus knockout, lambda light chain locus not knockout) were immunized with antigen A (10 mice). Beacon® Optofluidic System was then used to isolate the plasma cells that can produce antigen-specific monoclonal antibodies. The number of positive cells confirmed by FACS was 40. Further analysis of the antibody sequences showed a total of 14 unique CDR3 sequences. In addition, the affinity of some antibodies was also tested. As shown in FIG. 25, the KD of these antibodies against antigen A reached 10⁻⁸ M, indicating good binding affinity.

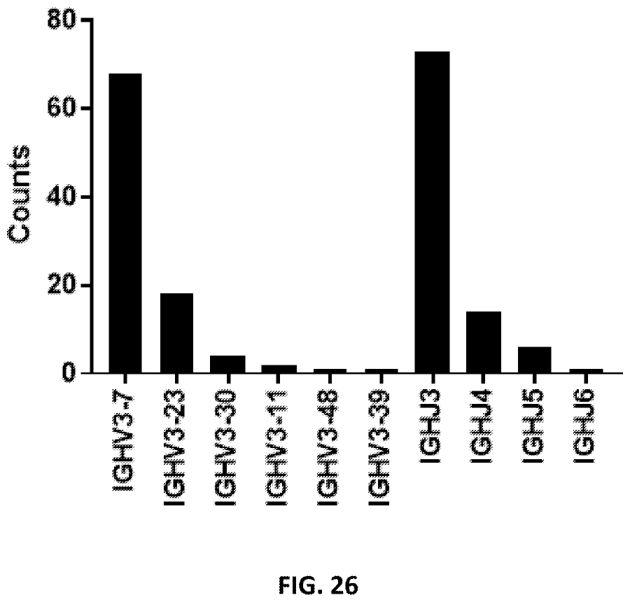

Further, spleen tissues of the immunized Mut2 mice were collected to extract total RNA from splenocytes. The immunoglobulin variable region locus can be cloned by PCR, and then inserted to phage plasmid to construct a phage recombinant plasmid library. In one experiment, after 2 rounds of panning and screening of the constructed library, a total of 202 ELISA positive clones were obtained. After removing redundant sequences, the number of positive cells confirmed by FACS was 94. Further analysis of the sequences of these antibodies showed that the CDR3 length of the heavy chain variable region was between 8-18 (defined by IMGT). FIG. 26 shows the germline gene usage of the variable region genes.

Mice with Mutant Allele 2′ (Mut2′)

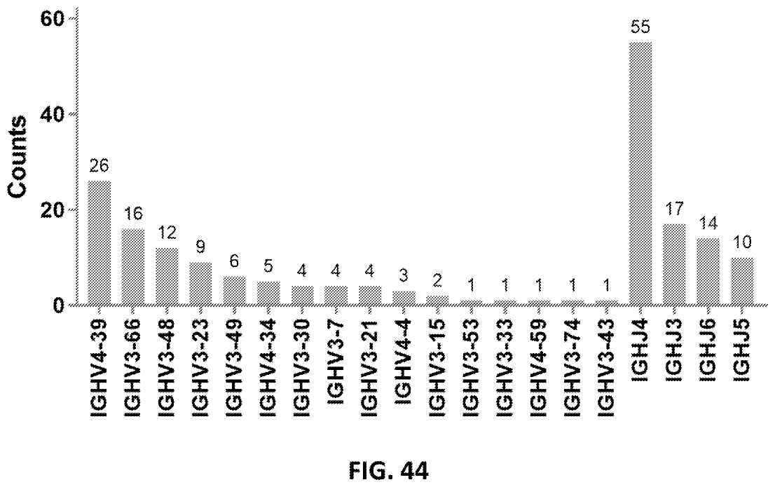

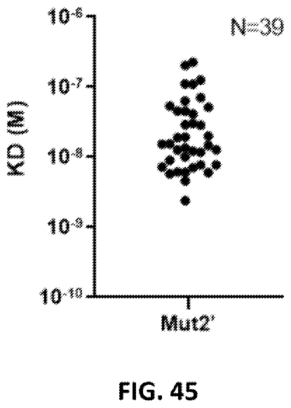

Humanized mice (heterozygous for heavy chain Mutant Allele 2′ genotype, kappa light chain and lambda light chain locus knockout) were immunized with antigen Human Serum Albumin (12mice). Beacon® Optofluidic System was then used to isolate the plasma cells that can produce antigen-specific monoclonal antibodies. The number of positive cells confirmed by FACS was 96. Further analysis of the antibody sequences showed that the CDR3 length of the heavy chain variable region (defined by IMGT) was between 5-22. In addition, 78.1% of the clones (75/96) had CDR3 lengths greater than or equal to 12. FIG. 44 shows the germline gene usage of the variable region genes. Further, the affinity of some antibodies was also tested. As shown in FIG. 45, the KD of some antibodies against Human Serum Albumin reached 10⁻⁹ M, indicating good binding affinity.

Mice with Mutant Allele 4 (Mut4)

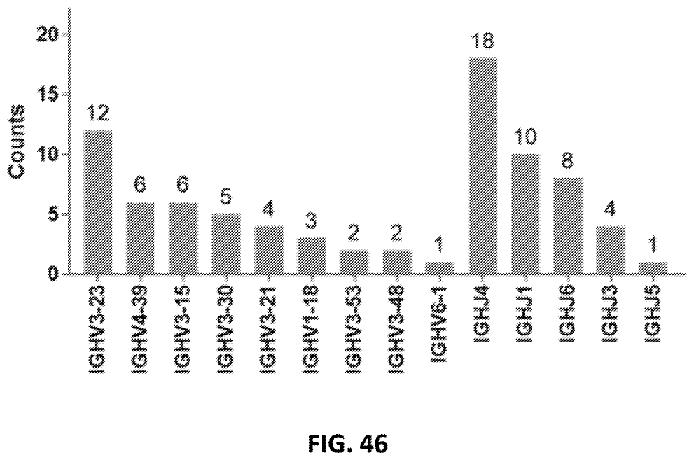

Humanized mice (homozygous for heavy chain Mutant Allele 4 genotype, kappa light chain and lambda light chain locus knockout) were immunized with antigen Human Serum Albumin (10 mice). Beacon® Optofluidic System was then used to isolate the plasma cells that can produce antigen-specific monoclonal antibodies. The number of positive cells confirmed by FACS was 41. Further analysis of the antibody sequences showed that the CDR3 length of the heavy chain variable region (defined by IMGT) was between 8-23, 65.9% of the clones (27/41) had CDR3 lengths greater than or equal to 12. FIG. 46 shows the germline gene usage of the variable region genes.

Mice with Mutant Allele 5 (Mut5)

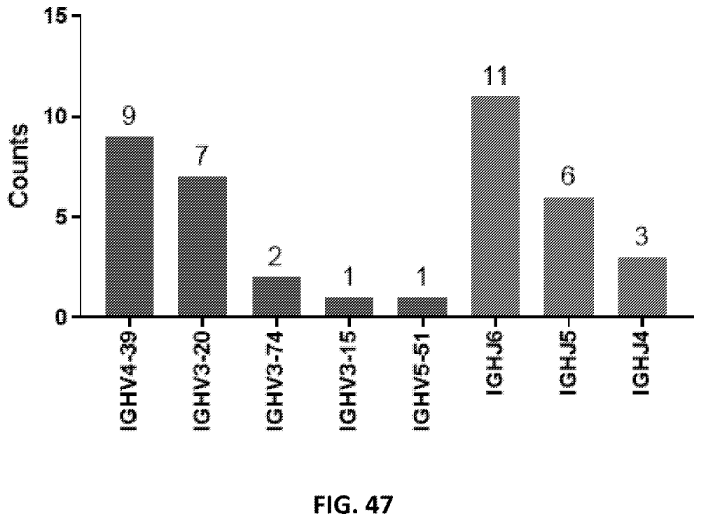

Humanized mice (homozygous for heavy chain Mutant Allele 5 genotype, kappa light chain and lambda light chain locus knockout) were immunized with antigen A. Beacon® Optofluidic System was then used to isolate the plasma cells that can produce antigen-specific monoclonal antibodies. The number of positive cells confirmed by FACS was 20. Further analysis of sequences showed that the CDR3 length of the heavy chain variable region (defined by IMGT) was between 10-18. FIG. 47 shows the germline gene usage of the variable region genes. In addition, the affinity of some antibodies was also tested. The KD of these antibodies against antigen A reached 109 M, indicating good binding affinity.

### B-Cell Development

Experiments were performed to compare the immune systems of the modified IgG1 mice and the wild-type mice. 6-8 week old RenMab mice (humanized heavy chain immunoglobulin locus), and modified IgG1 mice were selected. Among them, the modified IgG1 mice had similar body weight, appearance and vitality as compared to the RenMab mice. Peripheral blood, spleen, lymph nodes and bone marrow tissues of these mice were obtained, and no obvious anatomical changes were discovered.

### Example 8. Generating Human Anti-TFR1 Antibodies

The mice (5 mice were homozygous for heavy chain Mutant allele 3 genotype, homozygous for the deletion of kappa light chain locus, and heterozygous for the deletion of lambda light chain locus) were immunized with His-tagged human TFR1 (transferrin receptor 1) protein (hTFR1-His, ACROBiosystems, Cat #: CD1-H5243) to obtain anti-TFR1 antibodies. Before immunization, retro-orbital blood was collected as a negative control. Freund's complete adjuvant (CFA) was used for the first immunization and Freund's incomplete adjuvant (IFA) was used for the second and third immunizations. A total of three immunizations (bi-weekly) were performed. One week after the third immunization, retro-orbital blood was collected, and the antibody titer of serum was detected by FACS.

Procedures to enhance immunization were also performed at least fourteen days after the previous immunizations. TFR1 protein was injected by intraperitoneal injection, and the CHO-S cells expressing human TFR1 antigen was injected through the tail vein.

Antigen-specific immune cells were isolated from the immunized mice to further obtain anti-TFR1 antibodies or to obtain the heavy chain variable region sequences of the anti-TFR1 antibodies. For example, single cell technology (e.g., using Beacon® Optofluidic System, Berkeley Lights Inc.) was used to screen and find plasma cells that secrete antigen-specific monoclonal antibodies. Reverse transcription and PCR sequencing were used to obtain antibody variable region sequences. The obtained variable region sequences were used for antibody expression to verify the binding affinity to TFR1 using FACS. Because the lack of the CH1 domain, the heavy chain variable region (VH) of the obtained antibodies is also referred to as a heavy chain single variable domain (VHH).

Specifically, the obtained VHH sequences were respectively connected to a human IgG1 constant region (e.g., the hinge region, CH2 domain and CH3 domain). Exemplary antibodies obtained by this method included: 23B8, 24A1, 24C9 and 24G5. The heavy chain CDR1-3 sequences are shown in FIG. 37 and FIG. 38. The VHH region sequences of 23B8, 24A1, 24C9 and 24G5 are shown in FIG. 39.

The constant region of the antibodies can be further engineered to replace the Asparagine at position 297 with Alanine (N297A). For example, when N297A mutation is introduced into the constant region of 24G5, the resulting antibody is named as 24G5-N.

### Example 9. Cross-Species Binding of Anti-TFR1 Antibodies

CHO-S-hTFR1 cells or CHO-S-fasTFR1 cells were transferred to a 96-well plate at a density of 10⁵ cells/well respectively. Serially diluted sample anti-TFR1 antibodies were added to the 96-well plate, and incubated at 4° C. for 30 minutes. PBS was used as a negative control (NC). Then, the cells were incubated with the secondary antibody anti-hIgG-Fc-Alex Flour™ 647 (Jackson ImmunoResearch Laboratories, Cat #: 109-606-170) at 4° C. in the dark for 15 minutes before flow cytometry analysis.

CHO-S-hTFR1 cells or CHO-S-fasTFR1 cells were obtained by transfecting CHO-S cells with vectors expressing human TFR1 (hTFR1, SEQ ID NO: 70) or Macaca fascicularis (crab-eating macaque) TFR1 amino acid sequence (fasTFR1, SEQ ID NO: 71), respectively. The test results are shown in the table below.

JR141, a humanized IgG1 antibody targeting human TFR1 conjugated to human iduronate-2-sulfatase, was first approved in March 2021 in Japan for the intravenous treatment of mucopolysaccharidosis type II. The VH and VL sequences of JR141 are set forth in SEQ ID NO: 72 and SEQ ID NO: 73, respectively. For positive control (JR141-N), the VH and VL of JR141 were connected to a human IgG1 constant region with N297A mutation.

TABLE 15

| Antibody | Cells | Percentage of Positive Cells | Evaluation |
|---|---|---|---|
| NC | CHO-S-hTFR1 | 0.1% | No Binding |
| | CHO-S-fasTFR1 | 0.2% | No Binding |
| JR141-N | CHO-S-hTFR1 | 45.0% | Binding |
| | CHO-S-fasTFR1 | 37.2% | Binding |
| 23B8 | CHO-S-hTFR1 | 38.8% | Binding |
| | CHO-S-fasTFR1 | 39.6% | Binding |
| 24A1 | CHO-S-hTFR1 | 56.4% | Binding |
| | CHO-S-fasTFR1 | 50.9% | Binding |
| 24C9 | CHO-S-hTFR1 | 45.8% | Binding |
| | CHO-S-fasTFR1 | 0.35% | No Binding |
| 24G5 | CHO-S-hTFR1 | 54.3% | Binding |
| | CHO-S-fasTFR1 | 51.2% | Binding |

### Example 10. Binding Affinity of Anti-TFR1 Antibodies

The binding affinity of the anti-TFR1 antibodies to His-tagged TFR1 protein of human (hTFR1-His, ACROBiosystems, Cat #: CD1-H5243) or monkey (fasTFR1-His, ACROBiosystems, Cat #: TFR-C524a) were verified using surface plasmon resonance (SPR) on Biacore™ (Biacore, Inc., Piscataway N.J.) 8K biosensor equipped with pre-immobilized Protein A sensor chips.

Purified anti-TFR1 antibodies was captured on the Protein A chip (Series S Sensor Chip Protein A) for the detection. Purified anti-TFR1 antibodies (1 μg/mL) was loaded at 10 μL/min to bind to hTFR1-His and fasTFR1-His (200 nM). The flow rate was 30 μL/min. The binding and dissociation time were set to 180 seconds and 600 seconds, respectively. The chip was regenerated after the last injection of each titration with a glycine solution (pH 2.0) at 30 L/min for 30 seconds.

Kinetic association rates (kon) and dissociation rates (koff) were obtained simultaneously by fitting the data globally to a 1:1 Langmuir binding model (Karlsson, R. Roos, H. Fagerstam, L. Petersson, B., 1994. Methods Enzymology 6. 99-110) using Biacore™ 8K Evaluation Software 3.0. Affinities were deduced from the quotient of the kinetic rate constants (KD=koff/kon).

As a person of ordinary skill in the art would understand, the same method with appropriate adjustments for parameters (e.g., antibody concentration) was performed for each tested antibody. The results for the tested antibodies are summarized in the table below. The results show that all four anti-TFR1 antibodies can bind to human and monkey TFR1 with high affinity.

TABLE 16

| Protein | Analyte 1 Solution | kon (1/Ms) | koff (1/s) | KD (M) |
|---|---|---|---|---|
| 23B8 | hTFR1-His | 1.51E+04 | 1.32E−04 | 8.73E−09 |
| | fasTFR1-His | 4.10E+05 | 7.99E−03 | 1.95E−08 |
| 24A1 | hTFR1-His | 2.62E+04 | 7.68E−05 | 2.93E−09 |
| | fasTFR1-His | 4.36E+04 | 1.01E−04 | 2.32E−09 |
| 24C9 | hTFR1-His | 2.68E+04 | 3.83E−04 | 1.43E−08 |
| | fasTFR1-His | 2.78E+04 | 3.68E−04 | 1.32E−08 |
| 24G5 | hTFR1-His | 4.24E+04 | 8.68E−05 | 2.05E−09 |
| | fasTFR1-His | 6.33E+04 | 8.79E−05 | 1.39E−09 |

### Example 11. Epitope Analysis of Anti-TFR1 Antibodies

Relative positions of target protein epitope between a pair of purified anti-TFR1 antibodies were analyzed by Biolayer Interferometry (BLI) using ForteBio Octet system at 30° C. 1×HBS-EP+ buffer (10 mM 4-(2-hydroxyethyl)-1-piperazineethanesulfonic acid (HEPES), 150 mM NaCl, 3 mM ethylenediaminetetraacetic acid (EDTA) and 0.05% P20, pH7.4) diluted from HBS-EP+ buffer (10×) was used as the running buffer throughout the experiment. About 10 μg/mL of hTFR1-His protein was captured by HIS1K (Anti-Penta-HIS) for 200 seconds, and 200 nM of antibody (analyte 1) was injected at a flow rate of 30 μL/min to bind the ligand. Another antibody (analyte 2) was injected under the same conditions to determine whether the binding of different antibodies interfered with each other. The binding time was 300 seconds for each antibody.

The binding value of each antibody was obtained using Data Analysis HT 12.0. To quantify the interference of one antibody binding to another, a binding ratio was calculated to compare each pair of antibodies. The binding ratio is defined as the binding value of the second antibody (analyte 2), divided by the binding value of the first antibody (analyte 1). The binding ratio of each antibody pair is summarized in the matrix table below. Specifically, the binding ratio was between 0.0 to 0.5, if analyte 1 exhibited a blocking effect to analyte 2; the binding ratio was between 0.5-1.1, if analyte 1 did not exhibit a blocking effect to analyte 2. In general, antibody pairs that interfere with each other have the same or overlapping epitopes.

The epitope binding assay results indicate that 24A1 and 24G5 can recognize the same epitope, and 23B8, 24C9 and JR141-N can recognize different epitopes.

TABLE 17

Analyte 1

| Analyte 2 | 23B8 | 24A1 | 24G5 | 24C9 | JR141-N |
|---|---|---|---|---|---|
| 23B8 | 0.1 | 0.2 | 0.3 | 1.4 | 1.1 |
| 24A1 | 0.0 | 0.1 | 0.1 | 0.1 | 0.3 |
| 24G5 | 0.0 | 0.0 | 0.0 | 0.0 | 0.2 |
| 24C9 | 0.6 | 0.1 | 0.2 | 0.0 | 0.8 |
| JR141-N | 0.7 | 0.0 | 0.0 | 1.1 | 0.0 |

### Example 12. Internalization of Anti-TFR1 Antibodies

Anti-TFR1 antibodies together with the pHAb-Goat anti-human IgG secondary antibody were added to human cortical microvascular endothelial cells (hCMEC/D cells), and incubated for 3 hours. After incubation, the cells were centrifuged and washed with FACS buffer. Mean fluorescence intensity (MFI) was measured using a flow cytometer. Endocytosis rates of antibodies were calculated. Human IgG1 protein (CrownBio, Cat #: C0001) was used as an isotype control (ISO). The results are shown in the following table, indicating that all four antibodies exhibited good endocytosis activity in human cortical microvascular endothelial cells.

TABLE 18

| Antibody | MFI | Positive Population |
|---|---|---|
| ISO | 4288 | 0.9% |
| 23B8 | 11392 | 67.0% |
| 24A1 | 26972 | 96.6% |
| 24C9 | 37086 | 96.0% |
| 24G5 | 26406 | 96.0% |

### Example 13. Stability Analysis of Anti-TFR1 Antibodies

Stability of anti-TFR1 antibodies 23B8, 24A1, 24C9 and 24G5 was evaluated. Specifically, the following tests were performed: (1) observing the solution appearance and presence of visible non-soluble objects; (2) detecting the purity changes of antibodies by Size-Exclusion Ultra Performance Liquid Chromatography (SEC-UPLC) (indicated as the percentage of the main peak area to the sum of all peak areas (Purity, %)); (3) detecting changes in the apparent hydrophobicity of the antibodies using the Hydrophobic Interaction Chromatography-High Performance Liquid Chromatography (HIC-HPLC) method (indicated as the retention time of the main peak (HIC, min); (4) detecting charge variants in the antibodies by Capillary Isoelectric Focusing (cIEF) (indicated as the percentages of the main component, acidic component, and alkaline component); and (5) detecting the thermal stability of antibodies via the UNcle system (indicated as the melting temperature (Tm) and aggregation temperature (Tagg)).

In the SEC-UPLC experiments, the Agilent 1290 chromatograph system (connected with XBridge™ Protein BEH SEC column (200 Å, Waters Corporation)) was used. The antibody samples were diluted to 1 mg/mL with purified water. The following parameters were used: mobile phase: 25 mM phosphate buffer (PB) (pH 6.8)+0.3 M NaCl; flow rate: 1.8 mL/min; column temperature: 25° C.; detection wavelength: 280 nm; injection volume: 10 μL; sample tray temperature: 6° C.; and running time: 7 minutes.

In the HIC-HPLC experiments, the Agilent 1260 chromatograph system (connected with ProPac™ HIC-10 column (4.6×100 mm, Thermo Scientific)) was used, and samples were diluted using mobile phase A to 0.5 mg/mL. The following parameters were used: mobile phase A: 0.9 M ammonium sulfate, 0.1 M PB, 10% acetonitrile pH 6.5; mobile phase B: 0.1 M PB, 10% acetonitrile pH 6.5; flow rate: 0.8 mL/min; gradient: 0 min 100% A, 2 min 100% A, 32 min 100% B, 34 min 100% B, 35 min 100% A, and 45 min 100% A; column temperature: 30° C.; detection wavelength: 280 nm; injection volume: 10 μg; sample tray temperature: about 6° C.; and running time: 45 minutes.

In the cIEF experiments, the Maurice cIEF Method Development Kit (Protein Simple, Cat #: PS-MDK01-C) was used for sample preparation. Specifically, 40 μg protein sample was mixed with the following reagents in the kit: 1 μL Maurice cIEF pI Marker-4.05, 1 μL Maurice cIEF pI Marker-9.99, 35 μL 1% Methyl Cellulose Solution, 2 μL Maurice cIEF 500 mM Arginine, 4 μL Ampholytes (Pharmalyte pH ranges 3-10), and water (added to make a final volume of 100 μL). On the Maurice analyzer (Protein Simple, Santa Clara, CA), Maurice cIEF Cartridges (PS-MC02-C) were used to generate imaging capillary isoelectric focusing spectra. The sample was focused for a total of 10 minutes. The analysis software installed on the instrument was used to analyze the absorbance of the 280 nm-focused protein.

In the thermal stability experiments, antibody solutions at 60 mg/mL were heated from 25 to 95° C. using 1° C. increments, with an equilibration time of 1 minute before each measurement.

Detailed results are shown in the table below. The results indicate that all four antibodies have good stability and physical and chemical properties.

TABLE 19

| | SEC | HIC | | Tm | | | Tagg |
|---|---|---|---|---|---|---|---|
| Antibody | Purity (%) | (min) | PI | Tm1 (C) | Tm2 (° C.) | Tm3(° C.) | (° C.) |
| 23B8 | 92.82 | 19.909 | 7.376 | 58.87 | 67.80 | 84.13 | 56.48 |
| 24A1 | 98.74 | 2.486 | 6.795 | 64.80 | 73.80 | 82.20 | 52.73 |
| 24C9 | 98.20 | 3.740 | 8.595 | 65.38 | 69.90 | 78.20 | 33.63 |
| 24G5 | 98.86 | 3.829 | 6.495 | 60.86 | 77.66 | 85.30 | 76.03 |

### Example 14. Pharmacokinetics (PK) Analysis

A humanized TFR1 mouse model (hTFR1 mice) was engineered to express a chimeric TFR1 protein (SEQ ID NO: 74) in which the extracellular region of mouse TFR1 protein was replaced with the corresponding human TFR1 extracellular region. A detailed description regarding the humanized TFR1 mouse model can be found in PCT Application No. PCT/CN2022/105924, which is incorporated herein by reference in its entirety.

The concentrations of the anti-TFR1 antibodies were determined in hTFR1 mice. Specifically, the mice were placed into different groups (8 mice per group), and administered with an approximately equal molar dosage of JR141-N (G2), 23B8-N (G3), 24A1-N (G4), 24G5-N (G5), or 24C9-N (G6), by intravenous (i.v.) injection. The control group (G1) mice were administered with human IgG1 (hIgG1). Details of the administration scheme are shown in the table below.

TABLE 20

| | No. of | | | | |
|---|---|---|---|---|---|
| Group | mice | Antibodies | Dosage | Route | Frequency |
| G1 | 8 | hIgG1 | 18.4 mg/kg | i.v. | single dose |
| G2 | 8 | JR141-N | 18.4 mg/kg | i.v. | single dose |
| G3 | 8 | 23B8-N | 10.0 mg/kg | i.v. | single dose |
| G4 | 8 | 24A1-N | 10.0 mg/kg | i.v. | single dose |
| G5 | 8 | 24G5-N | 10.0 mg/kg | i.v. | single dose |
| G6 | 8 | 24C9-N | 10.0 mg/kg | i.v. | single dose |

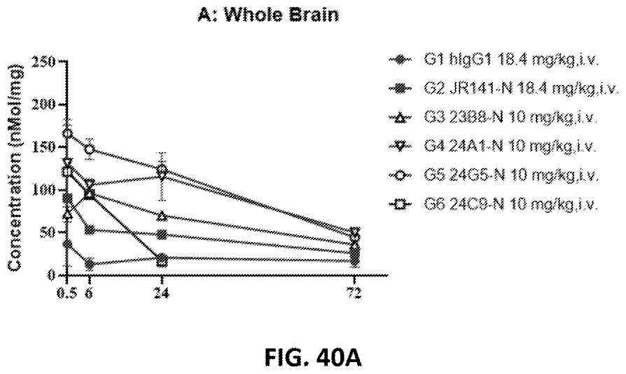

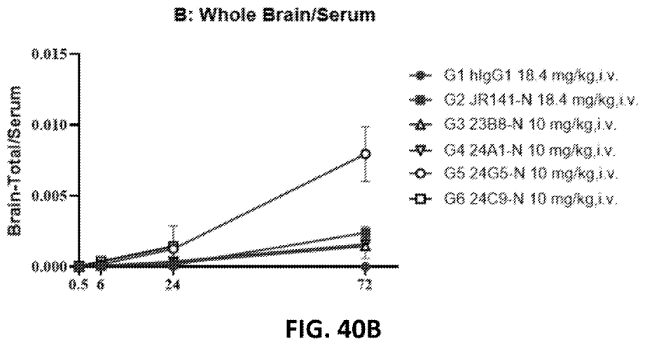

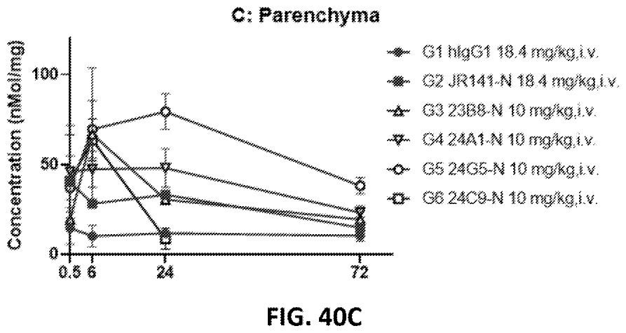

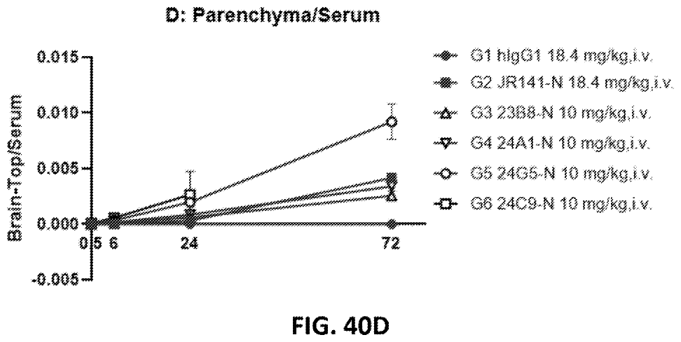

Blood and brain samples were collected 0.5, 6, 24, and 72 hours after the administration. Two mice were sampled at each time point, and the mice were anesthetized after retro-orbital blood was collected. To avoid interference from the residual blood in the brain, the mice were perfused by saline for 10 minutes at room temperature. Specifically, saline was perfused via systemic circulation from the left ventricle to the right ventricle. Brain samples were excised and divided into two hemibrains by the sagittal plane. The left hemibrain was subjected to quantification of the injected antibody, while the right hemibrain was fixed by formalin and embedded with paraffin for serial sections. The brain samples were cut into pieces and homogenized with DPBS (Dulbecco's phosphate-buffered saline) containing 1×mixed protease inhibitors. The brain homogenate was aliquoted for protein extraction followed by antibody quantification by electrochemiluminescence. For the rest homogenate, capillaries were depleted by gradient density centrifugation at 5400 g for 15 minutes, using 15% dextran. After centrifugation, the fraction at the top of the centrifuge tube was saved as parenchyma and subjected to protein extraction and antibody quantification as well. FIGS. 40A-40D show the antibody concentration in total brain protein (FIG. 40A), the ratio of antibody concentration in brain total protein to serum antibody concentration (FIG. 40B), the antibody concentration in brain parenchyma (FIG. 40C), and the ratio of antibody concentration in brain parenchyma to serum antibody concentration at each time point (FIG. 40D). These results demonstrated that 24G5-N (group G5) was the most abundant either in parenchyma or in the whole brain.

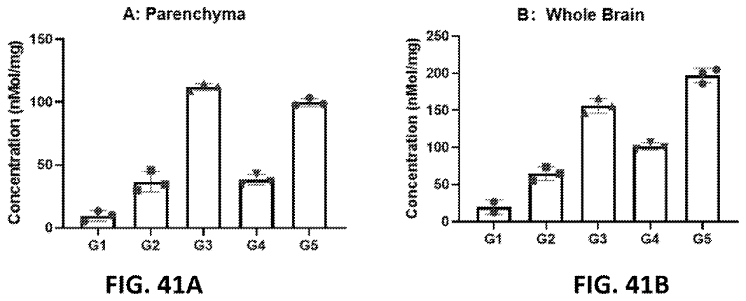

In a similar experiment, hTFR1 mice were placed into five groups (3 mice per group) and administered with 18.4 mg/kg JR141-N (G2), 10 mg/kg 23B8-N (G3), 10 mg/kg 24A1-N (G4), or 10 mg/kg 24G5-N (G5) by intravenous injection (1 administration in total). The control group (G1) mice were administered with hIgG1 (G1). 24 hours after administration, brain samples were collected to determine the concentrations of the anti-TFR1 antibodies. FIGS. 41A-41B show the antibody concentration test results in brain parenchyma and brain total protein, respectively. All tested antibodies showed a higher concentration in brain than hIgG1 (G1), indicating that compared with the positive control JR141-N (G2), 23B8-N (G3) and 24G5-N (G5) can better pass across the blood-brain barrier and enter the brain parenchyma.

In another similar experiment, hTFR1 mice were placed into seven groups (6 mice per group) and administered with JR141-N (G2-G4) or 24G5-N (G5-G7)) by intravenous (i.v.) injection. The control group (G1) mice were administered with hIgG1. Details of the administration scheme are shown in the table below.

TABLE 21

| | No. of | | | | | |
|---|---|---|---|---|---|---|
| Group | mice | Antibodies | Dosage (mg/kg) | | Route | Frequency |
| G1 | 6 | hIgG1 | 5.52 | mg/kg | i.v. | single dose |
| G2 | 6 | JR141-N | 1.84 | mg/kg | i.v. | single dose |
| G3 | 6 | JR141-N | 5.52 | mg/kg | i.v. | single dose |
| G4 | 6 | JR141-N | 18.4 | mg/kg | i.v. | single dose |
| G5 | 6 | 24G5-N | 1 | mg/kg | i.v. | single dose |
| G6 | 6 | 24G5-N | 3 | mg/kg | i.v. | single dose |
| G7 | 6 | 24G5-N | 10 | mg/kg | i.v. | single dose |

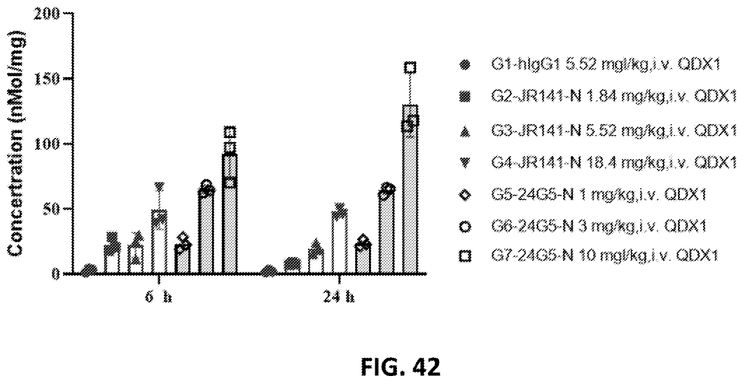

6 hours and 24 hours after the administration, blood and brain samples were collected using the methods described above. Three mice were sampled at each time point. Treatment of the tissues and quantification of antibodies were also performed as described above. The measurement results of the concentration of humanized anti-TFR1 antibodies in the brain parenchyma are shown in FIG. 42. The results show that the antibody concentration accumulated in the brain parenchyma of 24G5-N was significantly higher than that of hIgG1 under each dosage condition. Furthermore, the concentration of both JR141-N and 24G5-N exhibited a dose-dependent trend in brain parenchyma.

To detect the distribution of the humanized anti-TFR1 antibody 24G5-N in mouse brain, immunofluorescence assays were performed by staining hIgG, hTFR1, and mCD31, respectively, on right hemibrain sections of the mice used in the experiment above. The results showed that, mCD31 was well-labeled in the microvessels. hTFR1 was also detected on microvessels, which was co-localized with mCD31. Besides, hTFR1 expression was also detected on several neurons in parenchyma. In particular, the anti-TFR1 antibody 24G5-N was stained by a secondary anti-IgG antibody conjugated with DyLight® 488. Similar to hTFR1, 24G5-N was detected in microvessels and parenchyma, and its signal overlapped with the hTFR1 signal. Thus, either for the quantification of 24G5-N in the whole brain or parenchyma, or the visual evidence of immunofluorescence of 24G5-N in the parenchyma, the results indicate that humanized anti-TFR1 antibody 24G5-N can pass across the blood-brain barrier (BBB) efficiently.

### Example 15. Blocking Assay

Blockade of TFR1 binding to TF (transferrin) by anti-TFR1 antibodies 23B8, 24A1, 24C9 and 24G5 were tested by Biolayer Interferometry (BLI) using ForteBio Octet® system at 30° C. Specifically, 1×HBS-EP+ buffer (10 mM 4-(2-hydroxyethyl)-1-piperazineethanesulfonic acid (HEPES), 150 mM NaCl, 3 mM EDTA and 0.05% Surfactant P20, pH 7.4) diluted from HBS-EP+ buffer (10×) was used as the running buffer throughout the experiment. About 10 μg/mL of antibodies were captured by AHC (Anti-Human IgG Fc Capture) for 200 seconds, and 800 nM of hTFR1-His (ACROBiosystems, Cat #: CD1-H5243) and hTF-His (human transferrin protein, Kactus Biosystems, Cat #: TFN-HM101) were injected to bind the ligand. The binding time was 300 seconds for each antibody. The binding value of each antibody was obtained using Data Analysis HT 12.0. The results showed that these four antibodies did not block the binding of TFR1 to TF. Therefore, such non-blocking antibodies are not likely to interfere with TFR1-TF interactions in normal cells.

1. A genetically modified non-human animal comprising a modified immunoglobulin heavy chain locus, wherein the modified immunoglobulin heavy chain locus comprises an IgG constant region gene, wherein the IgG constant region gene encodes an IgG heavy chain constant region lacking a CH1 domain, wherein the genetically modified non-human animal expresses a heavy-chain antibody.

2. The genetically modified non-human animal of claim 1, wherein the animal comprises exactly one IgG constant region gene.

3. The genetically modified non-human animal of claim 1 or 2, wherein the IgG heavy chain constant region gene is IGHG1.

4. The genetically modified non-human animal of any one of claims 1-3, wherein the IgG heavy chain constant region comprises or consists of a CH2 domain and a CH3 domain, and optionally a hinge region.

5. A genetically modified non-human animal whose genome comprises a germline genetic modification comprising a deletion of IGHG3, IGHG2b, and IGHG2c genes and a deletion of the CH1 exon of IGHG1 gene at an endogenous immunoglobulin heavy chain gene locus.

6. The animal of claim 5, wherein the germline genetic modification further comprises a deletion of endogenous IGHE gene at the endogenous immunoglobulin heavy chain gene locus.

7. The animal of claim 5 or 6, wherein the genetic modification further comprises a deletion of endogenous Sγ2b, Sγ2c, and Sε switch regions at the endogenous immunoglobulin heavy chain gene locus.

8. The animal of any one of claims 5-7, wherein the modified immunoglobulin heavy chain gene locus comprises a modified IGHG1 gene lacking a sequence encoding a CH1 domain, wherein the modified IGHG1 gene comprises a sequence that is at least 80%, 90%, 95% or 99% identical to SEQ ID NO: 1.

9. The animal of any one of claims 5-8, wherein the genetic modification further comprises a deletion of endogenous Sγ3 switch region at the endogenous immunoglobulin heavy chain gene locus.

10. The animal of any one of claims 5-9, wherein the animal's genome comprises endogenous Sμ, Sγ1, Sα switch regions, the modified rodent IGHG1 gene lacking a sequence encoding a CH1 domain, and endogenous IGHM, IGHδ, IGHA genes.

11. The animal of any one of claims 5-9, wherein the genetic modification further comprises a deletion of endogenous IGHM and IGHδ genes at the endogenous immunoglobulin heavy chain gene locus.

12. The animal of any one of claims 5-9 and 11, wherein the animal genome comprises endogenous Sμ, Sγ1, Sα switch regions, the modified IGHG1 gene lacking a sequence encoding a CH1 domain, and the endogenous IGHA gene.

13. The animal of claim 12, wherein the Sμ and Sγ1 switch regions are linked with a sequence that is at least 80%, 90%, 95% or 99% identical to SEQ ID NO: 8.

14. The animal of any one of claims 5-9, wherein the genetic modification further comprises a deletion of the CH1 coding sequence of IGHM gene at the endogenous immunoglobulin heavy chain gene locus.

15. The animal of any one of claims 5-9 and 14, wherein the animal's genome comprises endogenous Sμ, Sγ1, Sα switch regions, a modified endogenous IGHM gene lacking a sequence encoding a CH1 domain, the modified IGHG1 gene lacking a sequence encoding a CH1 domain, and endogenous IGHδ, IGHA genes.

16. The animal of claim 15, wherein the Sμ switch region and the modified IGHM gene are linked with a sequence that is at least 80%, 90%, 95% or 99% identical to SEQ ID NO: 10.

17. The animal of any one of claims 5-9, wherein the genetic modification further comprises a deletion of the CH1 exon of IGHM gene and a deletion of IGHδ gene at the endogenous immunoglobulin heavy chain gene locus.

18. The animal of any one of claims 5-9 and 17, wherein the animal's genome comprises endogenous Sμ, Sγ1, Sα switch regions, a modified IGHM gene lacking a sequence encoding a CH1 domain, the modified IGHG1 gene lacking a sequence encoding a CH1 domain, and an endogenous IGHA gene.

19. The animal of any one of claims 5-9, wherein the genetic modification further comprises a deletion of the CH1 exon of IGHM gene and a deletion of the CH1 coding sequence of IGHδ gene at the endogenous immunoglobulin heavy chain gene locus.

20. The animal of any one of claims 5-9 and 19, wherein the animal's genome comprises endogenous Sμ, Sγ1, Sα switch regions, a modified IGHM gene lacking a sequence encoding a CH1 domain, a modified IGHδ gene lacking a sequence encoding a CH1 domain, the modified IGHG1 gene lacking a sequence encoding a CH1 domain, and an endogenous IGHA gene.

21. The animal of claim 19 or 20, wherein the modified IGHM gene are linked with a sequence that is at least 80%, 90%, 95% or 99% identical to SEQ ID NO: 10, and the modified IGHδ gene comprises a sequence that is at least 80%, 90%, 95%, or 99% identical to SEQ ID NO: 41.

22. The animal of any one of claims 14-21, wherein the modified IGHM gene comprises a sequence that is at least 80%, 90%, 95%, or 99% identical to SEQ ID NO: 13.

23. The animal of any one of claims 5-8, wherein the genetic modification further comprises a deletion of endogenous Sγ1 switch region at the endogenous immunoglobulin heavy chain gene locus.

24. The animal of any one of claims 5-8 and 23, wherein the animal's genome comprises endogenous Sμ, Sγ3, Sα switch regions, the modified IGHG1 gene lacking a sequence encoding a CH1 domain, and endogenous IGHM, IGHδ, IGHA genes.

25. The animal of any one of claims 5-8 and 23, wherein the genetic modification further comprises a deletion of endogenous Sγ3 switch region at the endogenous immunoglobulin heavy chain gene locus.

26. The animal of any one of claims 5-8, 23 and 25, wherein the genetic modification further comprises a deletion of endogenous IGHM and IGHδ genes at the endogenous immunoglobulin heavy chain gene locus.

27. The animal of any one of claims 5-8, 23, 25 or 26, wherein the animal's genome comprises endogenous Sμ, Sα switch regions, the modified IGHG1 gene lacking a sequence encoding a CH1 domain, and the endogenous IGHA gene.

28. The animal of claim 27, wherein the Sμ switch region and the modified IGHG1 gene are linked with a sequence that is at least 80%, 90%, 95%, or 99% identical to SEQ ID NO: 9.

29. The animal of any one of claims 5-10, 23 and 24, wherein the modified genome comprises a functional IGHM gene.

30. A genetically-modified non-human animal whose genome comprises the following elements, at an endogenous immunoglobulin heavy chain gene locus in a 5′ to 3′ order: Sμ switch region, IGHM gene, IGHδ gene, Sγ1 switch region, IGHG1 gene lacking a sequence encoding a CH1 domain, Sα switch region, and IGHA gene; wherein the elements are operably linked.

31. The animal of claim 30, wherein the endogenous immunoglobulin heavy chain constant region gene locus consists of the following functional genes and switch regions: Sμ switch region, IGHM gene, IGHδ gene, Sγ1 switch region, IGHG1 gene lacking a sequence encoding a CH1 domain, Sα switch region, and IGHA gene.

32. A genetically-modified non-human animal whose genome comprises the following elements, at an endogenous immunoglobulin heavy chain gene locus in a 5′ to 3′ order: Sμ switch region, IGHM gene, IGHδ gene, Sγ3 switch region, IGHG1 gene lacking a sequence encoding a CH1 domain, Sα switch region, and IGHA gene; wherein the elements are operably linked.

33. The animal of claim 32, wherein the endogenous immunoglobulin heavy chain constant region gene locus consists of the following functional genes and switch regions: Sμ switch region, IGHM gene, IGHδ gene, Sγ3 switch region, IGHG1 gene lacking a sequence encoding a CH1 domain, Sα switch region, and IGHA gene.

34. A genetically-modified non-human animal whose genome comprises the following elements, at an endogenous immunoglobulin heavy chain gene locus in a 5′ to 3′ order: Sμ switch region, Sγ1 switch region, IGHG1 gene lacking a sequence encoding a CH1 domain, Sα switch region, and IGHA gene; wherein the elements are operably linked.

35. The animal of claim 34, wherein the endogenous immunoglobulin heavy chain constant region gene locus consists of the following functional genes and switch regions: Sμ switch region, Sγ1 switch region, IGHG1 gene lacking a sequence encoding a CH1 domain, Sα switch region, and IGHA gene.

36. A genetically-modified non-human animal whose genome comprises the following elements, at an endogenous immunoglobulin heavy chain gene locus in a 5′ to 3′ order: Sμ switch region, IGHG1 gene lacking a sequence encoding a CH1 domain, Sα switch region, and IGHA gene; wherein the elements are operably linked.

37. The animal of claim 36, wherein the endogenous immunoglobulin heavy chain constant region gene locus consists of the following functional genes and switch regions: Sμ switch region, IGHG1 gene lacking a sequence encoding a CH1 domain, Sα switch region, and IGHA gene.

38. A genetically-modified non-human animal whose genome comprises the following elements, at an endogenous immunoglobulin heavy chain gene locus in a 5′ to 3′ order: Sμ switch region, IGHM gene lacking a sequence encoding a CH1 domain, IGHδ gene, Sγ1 switch region, IGHG1 gene lacking a sequence encoding a CH1 domain, Sα switch region, and IGHA gene; wherein the elements are operably linked.

39. The animal of claim 38, wherein the endogenous immunoglobulin heavy chain constant region gene locus consists of the following functional genes and switch regions: Sμ switch region, IGHM gene lacking a sequence encoding a CH1 domain, IGHδ gene, Sγ1 switch region, IGHG1 gene lacking a sequence encoding a CH1 domain, Sα switch region, and IGHA gene.

40. A genetically-modified non-human animal whose genome comprises the following elements, at an endogenous immunoglobulin heavy chain gene locus in a 5′ to 3′ order: Sμ switch region, IGHM gene lacking a sequence encoding a CH1 domain, Sγ1 switch region, IGHG1 gene lacking a sequence encoding a CH1 domain, Sα switch region, and IGHA gene; wherein the elements are operably linked.

41. The animal of claim 40, wherein the endogenous immunoglobulin heavy chain constant region gene locus consists of the following functional genes and switch regions: Sμ switch region, IGHM gene lacking a sequence encoding a CH1 domain, Sγ1 switch region, IGHG1 gene lacking a sequence encoding a CH1 domain, Sα switch region, and IGHA gene.

42. A genetically-modified non-human animal whose genome comprises the following elements, at an endogenous immunoglobulin heavy chain gene locus in a 5′ to 3′ order: Sμ switch region, IGHM gene lacking a sequence encoding a CH1 domain, IGHδ gene lacking a sequence encoding a CH1 domain, Sγ1 switch region, IGHG1 gene lacking a sequence encoding a CH1 domain, Sα switch region, and IGHA gene; wherein the elements are operably linked.

43. The animal of claim 42, wherein the endogenous immunoglobulin heavy chain constant region gene locus consists of the following functional genes and switch regions: Sμ switch region, IGHM gene lacking a sequence encoding a CH1 domain, IGHδ gene lacking a sequence encoding a CH1 domain, Sγ1 switch region, IGHG1 gene lacking a sequence encoding a CH1 domain, Sα switch region, and IGHA gene.

44. The animal of any one of claims 1-43, wherein the animal expresses a heavy-chain antibody comprising an IgG heavy chain constant region lacking the CH1 domain.

45. The animal of claim 44, wherein the heavy-chain antibody binds to it target antigen with a KD of less than 10⁻⁷ M, less than 10⁻⁸ M, or less than 10⁻⁹ M.

46. The animal of claim 44 or 45, wherein the heavy-chain antibody comprises or consists of a variable region, a CH2 domain and a CH3 domain.

47. The animal of any one of claims 44-46, wherein the heavy-chain antibody further comprises a transmembrane domain and/or a cytoplasmic domain.

48. The animal of any one of claims 1-47, wherein the genetically modified non-human animal does not express IgG antibodies comprising light chains.

49. The animal of any one of claims 1-48, wherein the animal expresses IgM, IgD, and/or IgA (e.g., functional IgM, IgD, and/or IgA).

50. The genetically modified non-human animal of any one of claims 1-49, wherein the animal comprises at an endogenous immunoglobulin heavy chain gene locus, one or more human IGHV genes, one or more human IGHD genes, and one or more human IGHJ genes, wherein the human IGHV genes, the human IGHD genes, and the human IGHJ genes are operably linked and can undergo VDJ rearrangement.

51. The genetically modified non-human animal of claim 50, wherein the animal comprises at least 150 human IGHV genes selected from Table 1, at least 20 human IGHD genes selected from Table 2, and at least 5 human IGHJ genes selected from Table 3.

52. The genetically modified non-human animal of claim 50, wherein the animal comprises all human IGHV genes, all human IGHD genes, and all human IGHJ genes at the endogenous immunoglobulin heavy chain gene locus of human chromosome 14 of a human subject.

53. The genetically modified non-human animal of claim 50, wherein the animal comprises all human IGHV genes, all human IGHD genes, and all human IGHJ genes at the endogenous immunoglobulin heavy chain gene locus of human chromosome 14 of a human cell.

54. The genetically modified non-human animal of any one of claim 50, wherein the animal is a mouse and the genetic modification in the animal's endogenous immunoglobulin heavy chain gene locus comprises a deletion of one or more mouse IGHV genes in Table 4, one or more mouse IGHD genes in Table 5, and/or one or more mouse IGHJ genes in Table 6.

55. The genetically modified non-human animal of claim 54, wherein the animal is a mouse and the genetic modification in the animal's endogenous heavy chain immunoglobulin gene locus comprises a deletion of a contiguous sequence starting from mouse IGHV1-85 gene to mouse IGHJ4 gene.

56. The genetically modified non-human animal of any one of claim 50, wherein the animal comprises an unmodified human sequence derived from a human heavy chain immunoglobulin gene locus, wherein the unmodified human sequence is at least 800 kb.

57. The genetically modified non-human animal of claim 50, wherein the animal comprises an unmodified human sequence derived from a human heavy chain immunoglobulin gene locus starting from human IGHV(III)-82 to human IGHV1-2.

58. The genetically modified non-human animal of claim 50, wherein the animal comprises an unmodified human sequence derived from a human heavy chain immunoglobulin gene locus starting from human IGHV(III)-82 to human IGHV6-1.

59. The genetically modified non-human animal of claim 50, wherein the animal comprises an unmodified human sequence derived from a human heavy chain immunoglobulin gene locus starting from human IGHD1-1 to human IGHJ6.

60. The genetically modified non-human animal of claim 50, wherein the animal comprises an unmodified human sequence derived from a human heavy chain immunoglobulin gene locus starting from human IGHV(III)-82 to human IGHJ6.

61. The genetically modified non-human animal of any one of claims 1-49, whose genome comprises, at the endogenous immunoglobulin heavy chain locus: a replacement of one or more endogenous IGHV, endogenous IGHD, and endogenous IGHJ genes with one or more human IGHV, human IGHD, and human IGHJ genes, wherein human IGHV, human IGHD, and human IGHJ genes are operably linked to one or more of endogenous IGHM, IGHδ, IGHG1 lacking a sequence encoding the CH1 domain, and IGHA genes.

62. The genetically modified non-human animal of claim 61, wherein one or more endogenous IGHV, endogenous IGHD, and endogenous IGHJ genes are replaced by at least 150 human IGHV genes in Table 1, at least 20 human IGHD genes in Table 2, and at least 5 human IGHJ genes in Table 3.

63. The genetically modified non-human animal of claim 61 or 62, wherein the animal is a mouse, and at least 180 mouse IGHV genes in Table 4, all mouse IGHD genes in Table 5, and all mouse IGHJ genes in Table 6 are replaced.

64. The genetically modified non-human animal of any one of claims 1-63, wherein the animal is homozygous with respect to the immunoglobulin heavy chain gene locus.

65. The genetically modified non-human animal of any one of claims 1-63, wherein the animal is heterozygous with respect to the immunoglobulin heavy chain gene locus.

66. The genetically modified non-human animal of any one of claims 1-65, wherein the animal comprises an endogenous light chain immunoglobulin gene locus.

67. The genetically modified non-human animal of any one of claims 1-65, wherein the animal comprises a disruption in the endogenous immunoglobulin light chain gene locus.

68. The genetically modified non-human animal of any one of claims 1-67, wherein the animal lacks an endogenous immunoglobulin heavy chain variable region locus that is capable of rearranging and forming a nucleic acid sequence that encodes an endogenous heavy chain variable domain.

69. The genetically modified non-human animal of any one of claims 1-68, wherein the animal can produce a humanized antibody.

70. The genetically modified non-human animal of any one of claims 1-53, 56-62, and 64-69, wherein the animal is a mammal.

71. The genetically modified non-human animal of claims 11-53, 56-62, and 64-70, wherein the animal is a rodent.

72. The genetically modified non-human animal of claims 1-53, 56-62, and 64-71, wherein the animal is a mouse.

73. The genetically modified non-human animal of any one of claims 1-72, wherein the animal has substantially normal B cell development and maturation.

74. A cell obtained from the genetically modified non-human animal of any one of claims 1-73.

75. The cell of claim 74, wherein the cell is a B cell that expresses a chimeric immunoglobulin heavy chain comprising an immunoglobulin heavy chain variable domain that is derived from a rearrangement of one or more human IGHV genes, one or more human IGHD genes, and one or more human IGHJ genes, wherein the immunoglobulin heavy chain variable domain is operably linked to a non-human heavy chain constant region.

76. The cell of claim 74 or 75, wherein the cell is an embryonic stem (ES) cell.

77. A method of making an antibody that specifically binds to an antigen, the method comprising
a) exposing the genetically modified non-human animal of any one of claims 1-73 to the antigen;
b) producing a hybridoma from a cell collected from the animal; and
c) collecting a heavy-chain antibody produced by the hybridoma.

78. The method of claim 77, wherein the method further comprises sequencing the genome of the hybridoma.

79. A method of making an antibody that specifically binds to an antigen, the method comprising
a) exposing the genetically modified non-human animal of any one of claims 1-73 to the antigen;
b) sequencing nucleic acids encoding human immunoglobulin heavy chain variable regions in a cell that expresses a heavy-chain antibody that specifically binds to the antigen; and
c) operably linking in a cell the nucleic acid encoding the human immunoglobulin heavy chain variable region with a nucleic acid encoding a human immunoglobulin heavy chain constant region.

80. A method of making an antibody that specifically binds to an antigen, the method comprising
a) obtaining a nucleic acid sequence encoding human immunoglobulin heavy chain variable regions in a cell that expresses a heavy-chain antibody that specifically binds to the antigen, wherein the cell is obtained by exposing the genetically modified non-human animal of any one of claims 1-73 to the antigen;
b) operably linking the nucleic acid encoding the human immunoglobulin heavy chain variable region with a nucleic acid encoding a human immunoglobulin heavy chain constant region; and
c) expressing the nucleic acid in a cell, thereby obtaining the antibody.

81. A method of obtaining a nucleic acid that encodes an antibody binding domain that specifically binds to an antigen, the method comprising
a) exposing the genetically modified non-human animal of any one of claims 1-73 to the antigen; and
b) sequencing nucleic acids encoding human immunoglobulin heavy chain variable regions in a cell that expresses a heavy-chain antibody that specifically binds to the antigen.

82. A method of making an antibody that specifically binds to antigen, the method comprising
a) exposing the genetically modified non-human animal of any one of claims 1-73 to the antigen;
b) constructing a phage plasmid library using RNA prepared from immune cells (e.g., splenocytes) of the animal;
c) screening the phage plasmid library; and
d) sequencing nucleic acids encoding human immunoglobulin heavy chain variable regions from a phage plasmid that encodes a heavy-chain antibody that specifically binds to the antigen.

83. The method of claim 82, wherein screening comprises isolating phages expressing immunoglobulin heavy chain variable regions based on binding affinity to the antigen.

84. A method of obtaining a sample, the method comprising
a) exposing the genetically modified non-human animal of any one of claims 1-73 to the antigen; and
b) collecting the sample from the animal.

85. The method of claim 84, wherein the sample is an immune cell, a lymphoid tissue, a spleen tissue, a spleen cell, or a B cell.
